SEQUENCE LISTING (197 sequences)

SEQ ID NO: 1 - protein, 897 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 2 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 3 - protein, 836 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 4 - protein, 420 aa, Homo sapiens
MIIVAHVLLILLGATEILQADLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 5 - protein, 108 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPITFGQGTRLEIKR

SEQ ID NO: 6 - protein, 108 aa, synthetic construct
DIQMTQSPSSVSASVGDRVTITCRASQGIRPFMAWYQQKPGKAPKLLIYAASSLQSGVPSRFSGSGSGTDFTLTISSLQPEDFATYYCQQANSFPITFGQGTRLEIKR

SEQ ID NO: 7 - protein, 108 aa, synthetic construct
DIQMTQSPSSVSASVGDRVTITCRASQGQRPFVSWYQQKPGKAPKLLIYAASSLQSGVPSRFSGSGSGTDFTLTISSLQPEDFATYYCQQANSFPITFGQGTRLEIKR

SEQ ID NO: 8 - protein, 108 aa, synthetic construct
DIQMTQSPSSVSASVGDRVTITCRASQGLRPFVNWYQQKPGKAPKLLIYAASSLQSGVPSRFSGSGSGTDFTLTISSLQPEDFATYYCQQANSFPITFGQGTRLEIKR

SEQ ID NO: 9 - protein, 108 aa, synthetic construct
DIQMTQSPSSVSASVGDRVTITCRASQGIRPFVDWYQQKPGKAPKLLIYAASSLQSGVPSRFSGSGSGTDFTLTISSLQPEDFATYYCQQANSFPITFGQGTRLEIKR

SEQ ID NO: 10 - protein, 108 aa, synthetic construct
DIQMTQSPSSVSASVGDRVTITCRASQGIRPFVAWYQQKPGKAPKLLIYAASSLQSGVPSRFSGSGSGTDFTLTISSLQPEDFATYYCQQANSFPITFGQGTRLEIKR

SEQ ID NO: 11 - protein, 108 aa, synthetic construct
DIQMTQSPSSVSASVGDRVTITCRASQGVRPFVNWYQQKPGKAPKLLIYAASSLQSGVPSRFSGSGSGTDFTLTISSLQPEDFATYYCQQANSFPITFGQGTRLEIKR

SEQ ID NO: 12 - protein, 108 aa, synthetic construct
DIQMTQSPSSVSASVGDRVTITCRASQGVRPFISWYQQKPGKAPKLLIYAASSLQSGVPSRFSGSGSGTDFTLTISSLQPEDFATYYCQQANSFPITFGQGTRLEIKR

SEQ ID NO: 13 - protein, 108 aa, synthetic construct
DIQMTQSPSSVSASVGDRVTITCRASQGIRPFIDWYQQKPGKAPKLLIYAASSLQSGVPSRFSGSGSGTDFTLTISSLQPEDFATYYCQQANSFPITFGQGTRLEIKR

SEQ ID NO: 14 - protein, 108 aa, synthetic construct
DIQMTQSPSSVSASVGDRVTITCRASQGVRPFVSWYQQKPGKAPKLLIYAASSLQSGVPSRFSGSGSGTDFTLTISSLQPEDFATYYCQQANSFPITFGQGTRLEIKR

SEQ ID NO: 15 - protein, 108 aa, synthetic construct
DIQMTQSPSSVSASVGDRVTITCRASQGIRPFVSWYQQKPGKAPKLLIYAASSLQSGVPSRFSGSGSGTDFTLTISSLQPEDFATYYCQQANSFPITFGQGTRLEIKR

SEQ ID NO: 16 - protein, 108 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCGKPVLDPITFGQGTRLEIKR

SEQ ID NO: 17 - protein, 108 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCGKPVFDPITFGQGTRLEIKR

SEQ ID NO: 18 - protein, 108 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCGIPVLGPITFGQGTRLEIKR

SEQ ID NO: 19 - protein, 108 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCGIPILGPITFGQGTRLEIKR

SEQ ID NO: 20 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 21 - protein, 117 aa, synthetic construct
EVQLLESGGGLVQPGGSLRLSCAASGFTFNHYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 22 - protein, 117 aa, synthetic construct
EVQLLESGGGLVQPGGSLRLSCAASGFTFMHYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 23 - protein, 117 aa, synthetic construct
EVQLLESGGGLVQPGGSLRLSCAASGFTFQWYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 24 - protein, 117 aa, synthetic construct
EVQLLESGGGLVQPGGSLRLSCAASGFTFEWYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 25 - protein, 117 aa, synthetic construct
EVQLLESGGGLVQPGGSLRLSCAASGFTFPWYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 26 - protein, 117 aa, synthetic construct
EVQLLESGGGLVQPGGSLRLSCAASGFTFIHYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 27 - protein, 117 aa, synthetic construct
EVQLLESGGGLVQPGGSLRLSCAASGFTFTHYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 28 - protein, 117 aa, synthetic construct
EVQLLESGGGLVQPGGSLRLSCAASGFTFVHYRVHWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 29 - protein, 117 aa, synthetic construct
EVQLLESGGGLVQPGGSLRLSCAASGFTFSHYHMLWVRQAPGKGLEWVSSISGAQWSTYYADSVKGRFTISRDNSKNTLYLQMNSLRAEDTAVYYCARFYDSFFDIWGQGTMVTVSS

SEQ ID NO: 30 - protein, 117 aa, synthetic construct
EVQLLESGGGLVQPGGSLRLSCAASGFTFSHYHMLWVRQAPGKGLEWVSSISGSVRSTYYADSVKGRFTISRDNSKNTLYLQMNSLRAEDTAVYYCARFYDSFFDIWGQGTMVTVSS

SEQ ID NO: 31 - protein, 117 aa, synthetic construct
EVQLLESGGGLVQPGGSLRLSCAASGFTFSHYHMLWVRQAPGKGLEWVSSINGSGWTTYYADSVKGRFTISRDNSKNTLYLQMNSLRAEDTAVYYCARFYDSFFDIWGQGTMVTVSS

SEQ ID NO: 32 - protein, 117 aa, synthetic construct
EVQLLESGGGLVQPGGSLRLSCAASGFTFSHYHMLWVRQAPGKGLEWVSSISGSHFPTYYADSVKGRFTISRDNSKNTLYLQMNSLRAEDTAVYYCARFYDSFFDIWGQGTMVTVSS

SEQ ID NO: 33 - protein, 117 aa, synthetic construct
EVQLLESGGGLVQPGGSLRLSCAASGFTFSHYHMLWVRQAPGKGLEWVSSINGFGRNTYYADSVKGRFTISRDNSKNTLYLQMNSLRAEDTAVYYCARFYDSFFDIWGQGTMVTVSS

SEQ ID NO: 34 - protein, 117 aa, synthetic construct
EVQLLESGGGLVQPGGSLRLSCAASGFTFSHYHMLWVRQAPGKGLEWVSSIGGDKRQTYYADSVKGRFTISRDNSKNTLYLQMNSLRAEDTAVYYCARFYDSFFDIWGQGTMVTVSS

SEQ ID NO: 35 - protein, 117 aa, synthetic construct
EVQLLESGGGLVQPGGSLRLSCAASGFTFSHYHMLWVRQAPGKGLEWVSSISGSMWSTYYADSVKGRFTISRDNSKNTLYLQMNSLRAEDTAVYYCARFYDSFFDIWGQGTMVTVSS

SEQ ID NO: 36 - protein, 117 aa, synthetic construct
EVQLLESGGGLVQPGGSLRLSCAASGFTFSHYHMLWVRQAPGKGLEWVSSINGMGATTYYADSVKGRFTISRDNSKNTLYLQMNSLRAEDTAVYYCARFYDSFFDIWGQGTMVTVSS

SEQ ID NO: 37 - protein, 117 aa, synthetic construct
EVQLLESGGGLVQPGGSLRLSCAASGFTFSHYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 38 - protein, 117 aa, synthetic construct
EVQLLESGGGLVQPGGSLRLSCAASGFTFSHYHMLWVRQAPGKGLEWVSSINGYGIQTYYADSVKGRFTISRDNSKNTLYLQMNSLRAEDTAVYYCARFYDSFFDIWGQGTMVTVSS

SEQ ID NO: 39 - protein, 117 aa, synthetic construct
EVQLLESGGGLVQPGGSLRLSCAASGFTFSHYHMLWVRQAPGKGLEWVSSINGKGWTTYYADSVKGRFTISRDNSKNTLYLQMNSLRAEDTAVYYCARFYDSFFDIWGQGTMVTVSS

SEQ ID NO: 40 - protein, 117 aa, synthetic construct
EVQLLESGGGLVQPGGSLRLSCAASGFTFSHYHMLWVRQAPGKGLEWVSSIGGDIRQTYYADSVKGRFTISRDNSKNTLYLQMNSLRAEDTAVYYCARFYDSFFDIWGQGTMVTVSS

SEQ ID NO: 41 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDHFDIWGQGTMVTVSS

SEQ ID NO: 42 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYDSHFDIWGQGTMVTVSS

SEQ ID NO: 43 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDNFDIWGQGTMVTVSS

SEQ ID NO: 44 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDHFREWGQGTMVTVSS

SEQ ID NO: 45 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDHFSPWGQGTMVTVSS

SEQ ID NO: 46 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDHFKPWGQGTMVTVSS

SEQ ID NO: 47 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDHFNPWGQGTMVTVSS

SEQ ID NO: 48 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYSDHFAPWGQGTMVTVSS

SEQ ID NO: 49 - protein, 117 aa, synthetic construct
EVQLLESGGGLVQPGGSLRLSCAASGFTFPWYRVHWVRQAPGKGLEWVSSIHTHRHITYYADSVKGRFTISRDNSKNTLYLQMNSLRAEDTAVYYCARFYDSFFDIWGQGTMVTVSS

SEQ ID NO: 50 - protein, 117 aa, synthetic construct
EVQLLESGGGLVQPGGSLRLSCAASGFTFPWYRVHWVRQAPGKGLEWVSSIHTHRNSTYYADSVKGRFTISRDNSKNTLYLQMNSLRAEDTAVYYCARFYDSFFDIWGQGTMVTVSS

SEQ ID NO: 51 - protein, 117 aa, synthetic construct
EVQLLESGGGLVQPGGSLRLSCAASGFTFPWYRVHWVRQAPGKGLEWVSSIHTGSQWTYYADSVKGRFTISRDNSKNTLYLQMNSLRAEDTAVYYCARFYDSFFDIWGQGTMVTVSS

SEQ ID NO: 52 - protein, 117 aa, synthetic construct
EVQLLESGGGLVQPGGSLRLSCAASGFTFPWYRVHWVRQAPGKGLEWVSSIHTHYQYTYYADSVKGRFTISRDNSKNTLYLQMNSLRAEDTAVYYCARFYDSFFDIWGQGTMVTVSS

SEQ ID NO: 53 - protein, 117 aa, synthetic construct
EVQLLESGGGLVQPGGSLRLSCAASGFTFPWYRVHWVRQAPGKGLEWVSSIHTQSKWTYYADSVKGRFTISRDNSKNTLYLQMNSLRAEDTAVYYCARFYDSFFDIWGQGTMVTVSS

SEQ ID NO: 54 - protein, 117 aa, synthetic construct
EVQLLESGGGLVQPGGSLRLSCAASGFTFPWYRVHWVRQAPGKGLEWVSSIRTDGTWTYYADSVKGRFTISRDNSKNTLYLQMNSLRAEDTAVYYCARFYDSFFDIWGQGTMVTVSS

SEQ ID NO: 55 - protein, 117 aa, synthetic construct
EVQLLESGGGLVQPGGSLRLSCAASGFTFPWYRVHWVRQAPGKGLEWVSSIQTHGVWTYYADSVKGRFTISRDNSKNTLYLQMNSLRAEDTAVYYCARFYDSFFDIWGQGTMVTVSS

SEQ ID NO: 56 - protein, 117 aa, synthetic construct
EVQLLESGGGLVQPGGSLRLSCAASGFTFPWYRVHWVRQAPGKGLEWVSSIHTHRNYTYYADSVKGRFTISRDNSKNTLYLQMNSLRAEDTAVYYCARFYDSFFDIWGQGTMVTVSS

SEQ ID NO: 57 - protein, 117 aa, synthetic construct; identical to SEQ ID NO: 56

SEQ ID NO: 58 - protein, 117 aa, synthetic construct
EVQLLESGGGLVQPGGSLRLSCAASGFTFPWYRVHWVRQAPGKGLEWVSSIKHGGRYTYYADSVKGRFTISRDNSKNTLYLQMNSLRAEDTAVYYCARFYDSFFDIWGQGTMVTVSS

SEQ ID NO: 59 - protein, 117 aa, synthetic construct
EVQLLESGGGLVQPGGSLRLSCAASGFTFPWYRVHWVRQAPGKGLEWVSSIHTHRPWTYYADSVKGRFTISRDNSKNTLYLQMNSLRAEDTAVYYCARFYDSFFDIWGQGTMVTVSS

SEQ ID NO: 60 - protein, 117 aa, synthetic construct
EVQLLESGGGLVQPGGSLRLSCAASGFTFPWYRVHWVRQAPGKGLEWVSSIHTHSDWTYYADSVKGRFTISRDNSKNTLYLQMNSLRAEDTAVYYCARFYDSFFDIWGQGTMVTVSS

SEQ ID NO: 61 - protein, 117 aa, synthetic construct
EVQLLESGGGLVQPGGSLRLSCAASGFTFPWYRVHWVRQAPGKGLEWVSSIHTHRQVTYYADSVKGRFTISRDNSKNTLYLQMNSLRAEDTAVYYCARFYDSFFDIWGQGTMVTVSS

SEQ ID NO: 62 - protein, 117 aa, synthetic construct
EVQLLESGGGLVQPGGSLRLSCAASGFTFPWYRVHWVRQAPGKGLEWVSSIRNENGWTYYADSVKGRFTISRDNSKNTLYLQMNSLRAEDTAVYYCARFYDSFFDIWGQGTMVTVSS

SEQ ID NO: 63 - protein, 117 aa, synthetic construct
EVQLLESGGGLVQPGGSLRLSCAASGFTFPWYRVHWVRQAPGKGLEWVSSIRSSGGFPYYHQKVKGRFTISRDNSKNTLYLQMNSLRAEDTAVYYCARFYDSFFDIWGQGTMVTVSS

SEQ ID NO: 64 - protein, 117 aa, synthetic construct
EVQLLESGGGLVQPGGSLRLSCAASGFTFPWYRVHWVRQAPGKGLEWVSSIRSSGGFPYYNYKVKGRFTISRDNSKNTLYLQMNSLRAEDTAVYYCARFYDSFFDIWGQGTMVTVSS

SEQ ID NO: 65 - protein, 117 aa, synthetic construct
EVQLLESGGGLVQPGGSLRLSCAASGFTFPWYRVHWVRQAPGKGLEWVSSIRSSGGFPYYNRRVKGRFTISRDNSKNTLYLQMNSLRAEDTAVYYCARFYDSFFDIWGQGTMVTVSS

SEQ ID NO: 66 - protein, 117 aa, synthetic construct
EVQLLESGGGLVQPGGSLRLSCAASGFTFPWYRVHWVRQAPGKGLEWVSSIRSSGGFPYYNHKVKGRFTISRDNSKNTLYLQMNSLRAEDTAVYYCARFYDSFFDIWGQGTMVTVSS

SEQ ID NO: 67 - protein, 117 aa, synthetic construct
EVQLLESGGGLVQPGGSLRLSCAASGFTFPWYRVHWVRQAPGKGLEWVSSIRSSGGFPYYHPSVKGRFTISRDNSKNTLYLQMNSLRAEDTAVYYCARFYDSFFDIWGQGTMVTVSS

SEQ ID NO: 68 - protein, 117 aa, synthetic construct
EVQLLESGGGLVQPGGSLRLSCAASGFTFPWYRVHWVRQAPGKGLEWVSSIRSSGGFPYYSLSVKGRFTISRDNSKNTLYLQMNSLRAEDTAVYYCARFYDSFFDIWGQGTMVTVSS

SEQ ID NO: 69 - protein, 117 aa, synthetic construct
EVQLLESGGGLVQPGGSLRLSCAASGFTFPWYRVHWVRQAPGKGLEWVSSIRSSGGFPHYNSVVKGRFTISRDNSKNTLYLQMNSLRAEDTAVYYCARFYDSFFDIWGQGTMVTVSS

SEQ ID NO: 70 - protein, 117 aa, synthetic construct
EVQLLESGGGLVQPGGSLRLSCAASGFTFPWYRVHWVRQAPGKGLEWVSSIRSSGGFPFYNSSVKGRFTISRDNSKNTLYLQMNSLRAEDTAVYYCARFYDSFFDIWGQGTMVTVSS

SEQ ID NO: 71 - protein, 117 aa, synthetic construct
EVQLLESGGGLVQPGGSLRLSCAASGFTFPWYRVHWVRQAPGKGLEWVSSIRSSGGFPYYNPFVKGRFTISRDNSKNTLYLQMNSLRAEDTAVYYCARFYDSFFDIWGQGTMVTVSS

SEQ ID NO: 72 - protein, 117 aa, synthetic construct
EVQLLESGGGLVQPGGSLRLSCAASGFTFPWYRVHWVRQAPGKGLEWVSSIRSSGGFPYYNNHVKGRFTISRDNSKNTLYLQMNSLRAEDTAVYYCARFYDSFFDIWGQGTMVTVSS

SEQ ID NO: 73 - protein, 117 aa, synthetic construct
EVQLLESGGGLVQPGGSLRLSCAASGFTFPWYRVHWVRQAPGKGLEWVSSIRSSGGFPMYNPHVKGRFTISRDNSKNTLYLQMNSLRAEDTAVYYCARFYDSFFDIWGQGTMVTVSS

SEQ ID NO: 74 - protein, 117 aa, synthetic construct
EVQLLESGGGLVQPGGSLRLSCAASGFTFPWYRVHWVRQAPGKGLEWVSSIRSSGGFTYYNPSVKNRFTISRDNSKNTLYLQMNSLRAEDTAVYYCARFYDSFFDIWGQGTMVTVSS

SEQ ID NO: 75 - protein, 117 aa, synthetic construct
EVQLLESGGGLVQPGGSLRLSCAASGFTFPWYRVHWVRQAPGKGLEWVSSIRSSGGFTYYNPAVRGRFTISRDNSKNTLYLQMNSLRAEDTAVYYCARFYDSFFDIWGQGTMVTVSS

SEQ ID NO: 76 - protein, 117 aa, synthetic construct
EVQLLESGGGLVQPGGSLRLSCAASGFTFPWYRVHWVRQAPGKGLEWVSSIRSSGGFTYYNPKVKHRFTISRDNSKNTLYLQMNSLRAEDTAVYYCARFYDSFFDIWGQGTMVTVSS

SEQ ID NO: 77 - protein, 117 aa, synthetic construct
EVQLLESGGGLVQPGGSLRLSCAASGFTFPWYRVHWVRQAPGKGLEWVSSIRSSGGFTYYNSEVKGRFTISRDNSKNTLYLQMNSLRAEDTAVYYCARFYDSFFDIWGQGTMVTVSS

SEQ ID NO: 78 - protein, 117 aa, synthetic construct
EVQLLESGGGLVQPGGSLRLSCAASGFTFPWYRVHWVRQAPGKGLEWVSSIRSSGGFTYYTPSVRGRFTISRDNSKNTLYLQMNSLRAEDTAVYYCARFYDSFFDIWGQGTMVTVSS

SEQ ID NO: 79 - protein, 117 aa, synthetic construct
EVQLLESGGGLVQPGGSLRLSCAASGFTFPWYRVHWVRQAPGKGLEWVSSIRSSGGFTYYNPSVMGRFTISRDNSKNTLYLQMNSLRAEDTAVYYCARFYDSFFDIWGQGTMVTVSS

SEQ ID NO: 80 - protein, 117 aa, synthetic construct
EVQLLESGGGLVQPGGSLRLSCAASGFTFFWFHHVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 81 - protein, 117 aa, synthetic construct
EVQLLESGGGLVQPGGSLRLSCAASGFTFYWYHMIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 82 - protein, 117 aa, synthetic construct
EVQLLESGGGLVQPGGSLRLSCAASGFTFNWFQHIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 83 - protein, 117 aa, synthetic construct
EVQLLESGGGLVQPGGSLRLSCAASGFTFPWFHTVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 84 - protein, 117 aa, synthetic construct
EVQLLESGGGLVQPGGSLRLSCAASGFTFQWYHYIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 85 - protein, 117 aa, synthetic construct
EVQLLESGGGLVQPGGSLRLSCAASGFTFQEFHTIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 86 - protein, 117 aa, synthetic construct
EVQLLESGGGLVQPGGSLRLSCAASGFTFFWFNTVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 87 - protein, 117 aa, synthetic construct
EVQLLESGGGLVQPGGSLRLSCAASGFTFQWFNIVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 88 - protein, 117 aa, synthetic construct
EVQLLESGGGLVQPGGSLRLSCAASGFTFPFYHKVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 89 - protein, 117 aa, synthetic construct
EVQLLESGGGLVQPGGSLRLSCAASGFTFPGYVWIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 90 - protein, 117 aa, synthetic construct
EVQLLESGGGLVQPGGSLRLSCAASGFTFQYFNVIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 91 - protein, 117 aa, synthetic construct
EVQLLESGGGLVQPGGSLRLSCAASGFTFQHYHTVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 92 - protein, 117 aa, synthetic construct
EVQLLESGGGLVQPGGSLRLSCAASGFTFQWYHVIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 93 - protein, 117 aa, synthetic construct
EVQLLESGGGLVQPGGSLRLSCAASGFTFHHYHTVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 94 - protein, 117 aa, synthetic construct
EVQLLESGGGLVQPGGSLRLSCAASGFTFHHFHVIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 95 - protein, 117 aa, synthetic construct
EVQLLESGGGLVQPGGSLRLSCAASGFTFPWFHFVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 96 - protein, 117 aa, synthetic construct
EVQLLESGGGLVQPGGSLRLSCAASGFTFEHYHTIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 97 - protein, 117 aa, synthetic construct
EVQLLESGGGLVQPGGSLRLSCAASGFTFQWFHYIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 98 - protein, 117 aa, synthetic construct
EVQLLESGGGLVQPGGSLRLSCAASGFTFQHFHWVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 99 - protein, 117 aa, synthetic construct
EVQLLESGGGLVQPGGSLRLSCAASGFTFPYYHMIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 100 - protein, 117 aa, synthetic construct
EVQLLESGGGLVQPGGSLRLSCAASGFTFMWYHHIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 101 - protein, 117 aa, synthetic construct
EVQLLESGGGLVQPGGSLRLSCAASGFTFVWYHHIWVRQAPGKGLEWVSSINGRGRVTYYADSVKGRFTISRDNSKNTLYLQMNSLRAEDTAVYYCARFYDSFFDIWGQGTMVTVSS

SEQ ID NO: 102 - protein, 117 aa, synthetic construct; identical to SEQ ID NO: 95

SEQ ID NO: 103 - protein, 117 aa, synthetic construct
EVQLLESGGGLVQPGGSLRLSCAASGFTFPYYHQVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 104 - protein, 117 aa, synthetic construct
EVQLLESGGGLVQPGGSLRLSCAASGFTFPWFVMVWVRQAPGKGLEWVSSINGRGRVTYYADSVKGRFTISRDNSKNTLYLQMNSLRAEDTAVYYCARFYDSFFDIWGQGTMVTVSS

SEQ ID NO: 105 - protein, 117 aa, synthetic construct
EVQLLESGGGLVQPGGSLRLSCAASRTSFMWYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 106 - protein, 117 aa, synthetic construct
EVQLLESGGGLVQPGGSLRLSCAASRGAPMWYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 107 - protein, 117 aa, synthetic construct
EVQLLESGGGLVQPGGSLRLSCAASDVDFGWYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 108 - protein, 117 aa, synthetic construct
EVQLLESGGGLVQPGGSLRLSCAASAGFFEYYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 109 - protein, 117 aa, synthetic construct
EVQLLESGGGLVQPGGSLRLSCAASPRWEWNYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 110 - protein, 117 aa, synthetic construct
EVQLLESGGGLVQPGGSLRLSCAASAYSEMWYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 111 - protein, 117 aa, synthetic construct
EVQLLESGGGLVQPGGSLRLSCAASGFTFSHYHMLWVRQAPGKGLEWVSSINGRGRVTHVAQEVKGRFTISRDNSKNTLYLQMNSLRAEDTAVYYCARFYDSFFDIWGQGTMVTVSS

SEQ ID NO: 112 - protein, 117 aa, synthetic construct
EVQLLESGGGLVQPGGSLRLSCAASGFTFSHYHMLWVRQAPGKGLEWVSSINGRGRVPQMAQEVKGRFTISRDNSKNTLYLQMNSLRAEDTAVYYCARFYDSFFDIWGQGTMVTVSS

SEQ ID NO: 113 - protein, 117 aa, synthetic construct
EVQLLESGGGLVQPGGSLRLSCAASGFTFSHYHMLWVRQAPGKGLEWVSSINGRGRVTYYQWPMNKRFTISRDNSKNTLYLQMNSLRAEDTAVYYCARFYDSFFDIWGQGTMVTVSS

SEQ ID NO: 114 - protein, 117 aa, synthetic construct
EVQLLESGGGLVQPGGSLRLSCAASGFTFSHYHMLWVRQAPGKGLEWVSSINGRGRVTYYNPAWKKRFTISRDNSKNTLYLQMNSLRAEDTAVYYCARFYDSFFDIWGQGTMVTVSS

SEQ ID NO: 115 - protein, 117 aa, synthetic construct
EVQLLESGGGLVQPGGSLRLSCAASGFTFSHYHMLWVRQAPGKGLEWVSSINGRGRVTYYARDHKKRFTISRDNSKNTLYLQMNSLRAEDTAVYYCARFYDSFFDIWGQGTMVTVSS

SEQ ID NO: 116 - protein, 117 aa, synthetic construct
EVQLLESGGGLVQPGGSLRLSCAASGFTFSHYHMLWVRQAPGKGLEWVSSINGRGRVTYYALEYKKRFTISRDNSKNTLYLQMNSLRAEDTAVYYCARFYDSFFDIWGQGTMVTVSS

SEQ ID NO: 117 - protein, 428 aa, Homo sapiens
WERSLAGAEETIPLQTLRCYADYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 118 - protein, 428 aa, Homo sapiens
WERSLAGAEETIPLQTLRCYNAYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 119 - protein, 428 aa, Homo sapiens
WERSLAGAEETIPLQTLRCYNDATSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 120 - protein, 428 aa, Homo sapiens
WERSLAGAEETIPLQTLRCYNDYASHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 121 - protein, 428 aa, Homo sapiens
WERSLAGAEETIPLQTLRCYNDYTAHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 122 - protein, 428 aa, Homo sapiens
WERSLAGAEETIPLQTLRCYNDYTSAITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 123 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQAFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 124 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSAVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 125 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFAVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 126 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVATDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 127 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVADVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 128 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTAVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 129 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDADYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 130 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVAIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 131 - protein, 444 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNAQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 132 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIAMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 133 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQAAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 134 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMAMRYEHIDHTFEIQYRKDTATWKDSKTETLQNAHSMALPALEPSTRYWARVRVRTSRTGYNGIWSEWSEARSWDTESHHHHHH

SEQ ID NO: 135 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKARYEHIDHTFEIQYRKDTATWKDSKTETLQNAHSMALPALEPSTRYWARVRVRTSRTGYNGIWSEWSEARSWDTESHHHHHH

SEQ ID NO: 136 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMAYEHIDHTFEIQYRKDTATWKDSKTETLQNAHSMALPALEPSTRYWARVRVRTSRTGYNGIWSEWSEARSWDTESHHHHHH

SEQ ID NO: 137 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRAEHIDHTFEIQYRKDTATWKDSKTETLQNAHSMALPALEPSTRYWARVRVRTSRTGYNGIWSEWSEARSWDTESHHHHHH

SEQ ID NO: 138 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYAHIDHTFEIQYRKDTATWKDSKTETLQNAHSMALPALEPSTRYWARVRVRTSRTGYNGIWSEWSEARSWDTESHHHHHH

SEQ ID NO: 139 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEAIDHTFEIQYRKDTATWKDSKTETLQNAHSMALPALEPSTRYWARVRVRTSRTGYNGIWSEWSEARSWDTESHHHHHH

SEQ ID NO: 140 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHADHTFEIQYRKDTATWKDSKTETLQNAHSMALPALEPSTRYWARVRVRTSRTGYNGIWSEWSEARSWDTESHHHHHH

SEQ ID NO: 141 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESHHHHHH

SEQ ID NO: 142 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSATGYNGIWSEWSEARSWDTESHHHHHH

SEQ ID NO: 143 - protein, 428 aa, Homo sapiens; identical to SEQ ID NO: 142

SEQ ID NO: 144 - protein, 444 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTAYNGIWSEWSEARSWDTESHHHHHH

SEQ ID NO: 145 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGANGIWSEWSEARSWDTESHHHHHH

SEQ ID NO: 146 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYAGIWSEWSEARSWDTESHHHHHH

SEQ ID NO: 147 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNAIWSEWSEARSWDTESHHHHHH

SEQ ID NO: 148 - protein, 428 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGAWSEWSEARSWDTESHHHHHH

SEQ ID NO: 149 - DNA, 41 bp, synthetic construct
  source 1..41: mol_type = other DNA
  misc_difference 18..23: note = n is any nucleotide provided that a codon comprising the n encoding an amino acid other than cysteine
gtcgtgcaagccagggtnnnnnntggtatcagcagaaaccg

SEQ ID NO: 150 - DNA, 45 bp, synthetic construct
  source 1..45: mol_type = other DNA
  misc_difference 22..27: note = n is any nucleotide provided that a codon comprising the n encoding an amino acid other than cysteine
gattttgcaacctattattgcnnnnnnccgattacctttggtcag

SEQ ID NO: 151 - DNA, 42 bp, synthetic construct
  source 1..42: mol_type = other DNA
  misc_difference 20..25: note = n is any nucleotide provided that a codon comprising the n encoding an amino acid other than cysteine
ctattattgccagcaggcannnnnntttggtcagggcacccg

SEQ ID NO: 152 - DNA, 42 bp, synthetic construct
  source 1..42: mol_type = other DNA
  misc_difference 20..25: note = n is any nucleotide provided that a codon comprising the n encoding an amino acid other than cysteine
agcaagcggttttacctttnnnnnntgggttcgtcaggcacc

SEQ ID NO: 153 - DNA, 44 bp, synthetic construct
  source 1..44: mol_type = other DNA
  misc_difference 20..25: note = n is any nucleotide provided that a codon comprising the n encoding an amino acid other than cysteine
ggaatgggttagcagcattnnnnnnacctattatgccgatagcg

SEQ ID NO: 154 - DNA, 43 bp, synthetic construct
  source 1..43: mol_type = other DNA
  misc_difference 20..25: note = n is any nucleotide provided that a codon comprising the n encoding an amino acid other than cysteine
agtttattattgcgcacgcnnnnnngatatttggggtcagggt

SEQ ID NO: 155 - DNA, 44 bp, synthetic construct
  source 1..44: mol_type = other DNA
  misc_difference 21..26: note = n is any nucleotide provided that a codon comprising the n encoding an amino acid other than cysteine
attattgcgcacgcttttatnnnnnntggggtcagggtacaatg

SEQ ID NO: 156 - DNA, 44 bp, synthetic construct; identical to SEQ ID NO: 153
  source 1..44: mol_type = other DNA
  misc_difference 20..25: note = n is any nucleotide provided that a codon comprising the n encoding an amino acid other than cysteine

SEQ ID NO: 157 - DNA, 44 bp, synthetic construct
  source 1..44: mol_type = other DNA
  misc_difference 19..24: note = n is any nucleotide provided that a codon comprising the n encoding an amino acid other than cysteine
cgtagcagcggtggttttnnnnnngtgaaaggtcgttttaccat

SEQ ID NO: 158 - DNA, 45 bp, synthetic construct
  source 1..45: mol_type = other DNA
  misc_difference 21..26: note = n is any nucleotide provided that a codon comprising the n encoding an amino acid other than cysteine
gcggtggttttacctattatnnnnnncgttttaccattagccgtg

SEQ ID NO: 159 - DNA, 42 bp, synthetic construct; identical to SEQ ID NO: 152
  source 1..42: mol_type = other DNA
  misc_difference 20..25: note = n is any nucleotide provided that a codon comprising the n encoding an amino acid other than cysteine

SEQ ID NO: 160 - DNA, 42 bp, synthetic construct
  source 1..42: mol_type = other DNA
  misc_difference 18..23: note = n is any nucleotide provided that a codon comprising the n encoding an amino acid other than cysteine
tgagctgtgcagcaagcnnnnnntatcatatgctgtgggttc

SEQ ID NO: 161 - DNA, 43 bp, synthetic construct
  source 1..43: mol_type = other DNA
  misc_difference 19..24: note = n is any nucleotide provided that a codon comprising the n encoding an amino acid other than cysteine
aatggtcgtggtcgtgttnnnnnngtgaaaggtcgttttacca

SEQ ID NO: 162 - DNA, 45 bp, synthetic construct
  source 1..45: mol_type = other DNA
  misc_difference 21..26: note = n is any nucleotide provided that a codon comprising the n encoding an amino acid other than cysteine
gtggtcgtgttacctattatnnnnnncgttttaccattagccgtg

SEQ ID NO: 163 - protein, 117 aa, synthetic construct
EVQLLESGGGLVQPGGSLRLSCAASGFTFXXXXXXWVRQAPGKGLEWVSSIRSSGGFTYYADSVKGRFTISRDNSKNTLYLQMNSLRAEDTAVYYCARFYDSFFDIWGQGTMVTVSS

SEQ ID NO: 164 - protein, 117 aa, synthetic construct
EVQLLESGGGLVQPGGSLRLSCAASGFTFSHYHMLWVRQAPGKGLEWVSSIXXXXXXTYYADSVKGRFTISRDNSKNTLYLQMNSLRAEDTAVYYCARFYDSFFDIWGQGTMVTVSS

SEQ ID NO: 165 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARXXXXXXDIWGQGTMVTVSS

SEQ ID NO: 166 - protein, 117 aa, synthetic construct
EVQLLESGGGLVQPGGSLRLSCAASGFTFSHYHMLWVRQAPGKGLEWVSSIRSSGGFTYYADSVKGRFTISRDNSKNTLYLQMNSLRAEDTAVYYCARFYXXXXXXWGQGTMVTVSS

SEQ ID NO: 167 - protein, 112 aa, synthetic construct
DIQMTQSPSSVSASVGDRVTITCRASQGXXXXXXWYQQKPGKAPKLLIYAASSLQSGVPSRFSGSGSGTDFTLTISSLQPEDFATYYCQQANSFPITFGQGTRLEIKRTVAA

SEQ ID NO: 168 - protein, 112 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCXXXXXXPITFGQGTRLEIKRTVAA

SEQ ID NO: 169 - protein, 112 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQAXXXXXXFGQGTRLEIKRTVAA

SEQ ID NO: 170 - protein, 117 aa, synthetic construct
EVQLLESGGGLVQPGGSLRLSCAASGFTFPWYRVHWVRQAPGKGLEWVSSIXXXXXXTYYADSVKGRFTISRDNSKNTLYLQMNSLRAEDTAVYYCARFYDSFFDIWGQGTMVTVSS

SEQ ID NO: 171 - protein, 117 aa, synthetic construct
EVQLLESGGGLVQPGGSLRLSCAASGFTFPWYRVHWVRQAPGKGLEWVSSIRSSGGFXXXXXXVKGRFTISRDNSKNTLYLQMNSLRAEDTAVYYCARFYDSFFDIWGQGTMVTVSS

SEQ ID NO: 172 - protein, 117 aa, synthetic construct
EVQLLESGGGLVQPGGSLRLSCAASGFTFPWYRVHWVRQAPGKGLEWVSSIRSSGGFTYYXXXXXXRFTISRDNSKNTLYLQMNSLRAEDTAVYYCARFYDSFFDIWGQGTMVTVSS

SEQ ID NO: 173 - protein, 117 aa, synthetic construct
EVQLLESGGGLVQPGGSLRLSCAASGFTFXXXXXXWVRQAPGKGLEWVSSINGRGRVTYYADSVKGRFTISRDNSKNTLYLQMNSLRAEDTAVYYCARFYDSFFDIWGQGTMVTVSS

SEQ ID NO: 174 - protein, 117 aa, synthetic construct
EVQLLESGGGLVQPGGSLRLSCAASXXXXXXYHMLWVRQAPGKGLEWVSSINGRGRVTYYADSVKGRFTISRDNSKNTLYLQMNSLRAEDTAVYYCARFYDSFFDIWGQGTMVTVSS

SEQ ID NO: 175 - protein, 117 aa, synthetic construct
EVQLLESGGGLVQPGGSLRLSCAASGFTFSHYHMLWVRQAPGKGLEWVSSINGRGRVXXXXXXVKGRFTISRDNSKNTLYLQMNSLRAEDTAVYYCARFYDSFFDIWGQGTMVTVSS

SEQ ID NO: 176 - protein, 117 aa, synthetic construct
EVQLLESGGGLVQPGGSLRLSCAASGFTFSHYHMLWVRQAPGKGLEWVSSINGRGRVTYYXXXXXXRFTISRDNSKNTLYLQMNSLRAEDTAVYYCARFYDSFFDIWGQGTMVTVSS

SEQ ID NO: 177 - protein, 108 aa, synthetic construct
  VARIANT 29: note = X is I, Q, L or V
  VARIANT 30: note = X is S or R
  VARIANT 31: note = X is S or P
  VARIANT 32: note = X is W or F
  VARIANT 33: note = X is L, M, V or I
  VARIANT 34: note = X is A, S, N or D
  VARIANT 89: note = X is Q or G
  VARIANT 90: note = X is Q, K or I
  VARIANT 91: note = X is A or P
  VARIANT 92: note = X is N, V or I
  VARIANT 93: note = X is S, L or F
  VARIANT 94: note = X is F, D or G
DIQMTQSPSSVSASVGDRVTITCRASQGXXXXXXWYQQKPGKAPKLLIYAASSLQSGVPSRFSGSGSGTDFTLTISSLQPEDFATYYCXXXXXXPITFGQGTRLEIKR

SEQ ID NO: 178 - protein, 11 aa, synthetic construct
  VARIANT 6: note = X is I, Q, L or V
  VARIANT 7: note = X is S or R
  VARIANT 8: note = X is S or P
  VARIANT 9: note = X is W or F
  VARIANT 10: note = X is L, M, V or I
  VARIANT 11: note = X is A, S, N or D
RASQGXXXXXX

SEQ ID NO: 179 - skipped

SEQ ID NO: 180 - protein, 117 aa, synthetic construct
  VARIANT 30: note = X is S, N, M, Q, E, P, I, T or V
  VARIANT 31: note = X is H or W
  VARIANT 33: note = X is H or R
  VARIANT 34: note = X is M or V
  VARIANT 35: note = X is L or H
  VARIANT 52: note = X is R, S, N or G
  VARIANT 53: note = X is S or G
  VARIANT 54: note = X is S, A, F, D, M, R, Y or K
  VARIANT 55: note = X is G, Q, V, H, K, M or I
  VARIANT 56: note = X is G, W, R, F, W, A or I
  VARIANT 57: note = X is F, S, T, P, N, Q or V
  VARIANT 101: note = X is D or S
  VARIANT 102: note = X is S or D
  VARIANT 103: note = X is F, H or N
  VARIANT 105: note = X is D, R, S, K, N or A
  VARIANT 106: note = X is I, E or P
EVQLLESGGGLVQPGGSLRLSCAASGFTFXXYXXXWVRQAPGKGLEWVSSIXXXXXXTYYADSVKGRFTISRDNSKNTLYLQMNSLRAEDTAVYYCARFYXXXFXXWGQGTMVTVSS

SEQ ID NO: 181 - protein, 10 aa, synthetic construct
  VARIANT 5: note = X is S, N, M, Q, E, P, I, T or V
  VARIANT 6: note = X is H or W
  VARIANT 8: note = X is H or R
  VARIANT 9: note = X is M or V
  VARIANT 10: note = X is L or H
GFTFXXYXXX

SEQ ID NO: 182 - protein, 17 aa, synthetic construct
  VARIANT 3: note = X is R, S, N or G
  VARIANT 4: note = X is S or G
  VARIANT 5: note = X is S, A, F, D, M, R, Y or K
  VARIANT 6: note = X is G, Q, V, H, K, M or I
  VARIANT 7: note = X is G, W, R, F, W, A or I
  VARIANT 8: note = X is F, S, T, P, N, Q or V
SIXXXXXXTYYADSVKG

SEQ ID NO: 183 - skipped

SEQ ID NO: 184 - protein, 117 aa, synthetic construct
  VARIANT 52: note = X is R, H, Q or K
  VARIANT 53: note = X is S, T, H or N
  VARIANT 54: note = X is S, H, G, Q, D or E
  VARIANT 55: note = X is G, R, S, Y or N
  VARIANT 56: note = X is G, H, N, Q, K, T, V, R, P or D
  VARIANT 57: note = X is F, I, S, W, V or Y
  VARIANT 58: note = X is T or P
  VARIANT 59: note = X is Y, H, F or M
  VARIANT 61: note = X is A, H, N, S or T
  VARIANT 62: note = X is D, Q, Y, R, H, P, L, S or N
  VARIANT 63: note = X is S, K, R, V, F, H, A or E
  VARIANT 65: note = X is K, R or M
  VARIANT 66: note = X is G, N or H
EVQLLESGGGLVQPGGSLRLSCAASGFTFPWYRVHWVRQAPGKGLEWVSSIXXXXXXXXYXXXVXXRFTISRDNSKNTLYLQMNSLRAEDTAVYYCARFYDSFFDIWGQGTMVTVSS

SEQ ID NO: 185 - protein, 10 aa, synthetic construct
GFTFPWYRVH

SEQ ID NO: 186 - protein, 17 aa, synthetic construct
  VARIANT 3: note = X is R, H, Q or K
  VARIANT 4: note = X is S, T, H or N
  VARIANT 5: note = X is S, H, G, Q, D or E
  VARIANT 6: note = X is G, R, S, Y or N
  VARIANT 7: note = X is G, H, N, Q, K, T, V, R, P or D
  VARIANT 8: note = X is F, I, S, W, V or Y
  VARIANT 9: note = X is T or P
  VARIANT 10: note = X is Y, H , F or M
  VARIANT 12: note = X is A, H, N, S or T
  VARIANT 13: note = X is D, Q, Y, R, H, P, L, S or N
  VARIANT 14: note = X is S, K, R, V, F, H, A or E
  VARIANT 16: note = X is K, R or M
  VARIANT 17: note = X is G, N or H
SIXXXXXXXXYXXXVXX

SEQ ID NO: 187 - protein, 8 aa, synthetic construct
FYDSFFDI

SEQ ID NO: 188 - protein, 117 aa, synthetic construct
  VARIANT 26: note = X is G, R, D, A or P
  VARIANT 27: note = X is F, G, V, R or Y
  VARIANT 28: note = X is T, S, A, P, F or W
  VARIANT 29: note = X is F, P or E
  VARIANT 30: note = X is S, F, Y, N, P, Q, H, E, M, V, G, W or M
  VARIANT 31: note = X is H, W, E, F, G, Y or N
  VARIANT 32: note = X is Y or F
  VARIANT 33: note = X is H, M, Q, N or V
  VARIANT 34: note = X is M, H, T, Y, I, K, W, V, F or Q
  VARIANT 35: note = X is L, V or I
  VARIANT 59: note = X is Y, H or Q
  VARIANT 60: note = X is Y, V or M
  VARIANT 61: note = X is A, Q or N
  VARIANT 62: note = X is D, Q, W, P, R or L
  VARIANT 63: note = X is S, E, P, A or D
  VARIANT 64: note = X is V, M, W, H or Y
  VARIANT 65: note = X is K or N
  VARIANT 66: note = X is G or K
EVQLLESGGGLVQPGGSLRLSCAASXXXXXXXXXXWVRQAPGKGLEWVSSINGRGRVTXXXXXXXXRFTISRDNSKNTLYLQMNSLRAEDTAVYYCARFYDSFFDIWGQGTMVTVSS

SEQ ID NO: 189 - skipped

SEQ ID NO: 190 - protein, 17 aa, synthetic construct
  VARIANT 10: note = X is Y, H or Q
  VARIANT 11: note = X is Y, V or M
  VARIANT 12: note = X is A, Q or N
  VARIANT 13: note = X is D, Q, W, P, R or L
  VARIANT 14: note = X is S, E, P, A or D
  VARIANT 15: note = X is V, M, W, H or Y
  VARIANT 16: note = X is K or N
  VARIANT 17: note = X is G or K
SINGRGRVTXXXXXXXX

SEQ ID NO: 191 - protein, 8 aa, synthetic construct
FYDSFFDI

SEQ ID NO: 192 - protein, 428 aa, Homo sapiens; identical to SEQ ID NO: 141

SEQ ID NO: 193 - protein, 117 aa, synthetic construct; identical to SEQ ID NO: 184

SEQ ID NO: 194 - protein, 444 aa, synthetic construct
EVQLLESGGGLVQPGGSLRLSCAASGFTFPWYRVHWVRQAPGKGLEWVSSIRSSGGFPYYNYKVKGRFTISRDNSKNTLYLQMNSLRAEDTAVYYCARFYDSFFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 195 - protein, 214 aa, synthetic construct
DIQMTQSPSSVSASVGDRVTITCRASQGLRPFVNWYQQKPGKAPKLLIYAASSLQSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 197 - protein, 104 aa, synthetic construct
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0152563 A1
(43) Pub. Date: Jun. 4, 2026

(54) Method of Treating Severe Asthma

(71) Applicant: CSL Innovation PTY LTD, Melbourne, Victoria (AU)

(72) Inventors: Steven Bozinovski, Melbourne (AU); Catherine Mary Owczarek, Melbourne (AU); Angel Francisco Lopez, Adelaide (AU); Hao Wang, Melbourne (AU); Damon John Tumes, Adelaide (AU)

(73) Assignee: CSL Innovation PTY LTD, Melbourne, Victoria (AU)

(21) Appl. No.: 19/122,543

(22) PCT Filed: Mar. 16, 2023
(86) PCT No.: PCT/AU2023/050187

§ 371 Date: Apr. 18, 2025

(30) Foreign Application Priority Data
Oct. 20, 2022 (AU) 2022903098

(51) Int. Cl. C07K 16/28; A61K 9/00; A61K 38/00; A61P 11/06

(52) U.S. Cl. CPC C07K 16/2866; A61K 9/0019; A61K 38/00; A61P 11/06; C07K 2317/565; C07K 2317/76

(57) ABSTRACT

The present disclosure relates to methods of treating severe asthma in a subject using compounds that bind to CD131 and inhibit signalling of interleukin (IL) 5 and granulocyte-macrophage colony stimulating factor (GM-CSF).

### RELATED APPLICATION DATA

The present application claims priority from Australian Patent Application No. 2022903098 filed 20 Oct. 2022 entitled “Method of treating severe asthma” the entire contents of which are hereby incorporated by reference.

### SEQUENCE LISTING

The present application is filed together with a Sequence Listing in electronic form. The entire contents of the Sequence Listing are hereby incorporated by reference.

### FIELD

The present disclosure relates to methods of treating a subject suffering from severe asthma using compounds that bind to CD131 and inhibit signalling of interleukin (IL) 5 and granulocyte-macrophage colony stimulating factor (GM-CSF).

### BACKGROUND

Asthma is a common chronic respiratory condition characterized by variable and recurring symptoms, reversible airway obstruction, airway (e.g., bronchial) hyper-responsiveness, and an underlying inflammation. Acute symptoms of asthma include cough, wheezing, shortness of breath and nocturnal awakening. These symptoms usually arise from bronchospasm and require and respond to bronchodilator therapy. Central to the pathophysiology of asthma is the presence of underlying airway inflammation mediated by the recruitment and activation of multiple cell types including mast cells, eosinophils, neutrophils, T lymphocytes, macrophages, and dendritic cells. The mechanisms influencing airway hyper-responsiveness are multiple and include inflammation, dysfunctional neuroregulation, and airway remodelling. Airway remodelling involves structural changes including thickening of the sub-basement membrane, subepithelial fibrosis, airway smooth muscle hypertrophy and hyperplasia, blood vessel proliferation and dilation with consequent permanent changes in the airway that increase airflow obstruction.

In most cases, asthma is treated using medication aimed at controlling the symptoms. These medications include compounds of various chemical and therapeutical classes, such as, for example, anti-inflammatory substances, leukotriene inhibitors, bronchodilators, cromolyn sodium and amino- or theophylline. However, in around 5-10% of patients, the asthma remains uncontrolled despite the use of high dose controller medications. Such patients are classified as having difficult to treat or severe asthma, which often requires a medical evaluation and may require hospitalization, oxygen, and intravenous medications. In addition to corticosteroids, several monoclonal antibodies have been developed (e.g., mepolizumab and benralizumab), however their clinical efficacy has been limited, possibly due to these antibodies targeting only individual cytokines and/or cytokines associated largely with the type 2 (T_{H}2) or eosinophilic inflammatory response (e.g., immunoglobulin E (IgE), interleukin (IL)-5, IL-4/IL-13 and thymic stromal lymphopoietin (TSLP)). Furthermore, asthma is a heterogeneous condition with over 50% of the patients with severe disease presenting with neutrophil-dominant or mixed granulocytic inflammation rather than eosinophilic/T_{H}2 inflammation. Currently available T_{H}2/eosinophilic therapies are insufficient to manage these other phenotypes, however no other clinical therapies currently exist.

Thus, there remains a need for improved therapies for treating asthma, particularly severe asthma, to better control the symptoms and to ameliorate the underlying disease processes.

### SUMMARY

In producing the present invention, the inventors identified CD131 (the R common receptor) as a potential target for pharmacological intervention of eosinophilic and/or neutrophilic inflammation associated with severe asthma. The inventors found that inhibition of interleukin (IL) 5 and granulocyte-macrophage colony stimulating factor (GM-CSF), using an antibody that binds to CD131, successfully reduced several measures of lung inflammation, airway hyper-responsiveness, and airway remodelling, and successfully improved measures of pulmonary function in an animal model of severe asthma.

The findings by the inventors provide the basis for methods of treating severe asthma in a subject.

Accordingly, the present disclosure provides a method of treating severe asthma in a subject, the method comprising administering one or more compound(s) that neutralize signaling by granulocyte-macrophage colony stimulating factor (GM-CSF) and interleukin (IL) 5 to the subject. The present disclosure also provides one or more compound(s) that neutralize signaling by GM-CSF and IL-5 for use in treating severe asthma in a subject. The present disclosure further provides use of one or more compound(s) that neutralize signaling by GM-CSF and IL-5 in the manufacture of a medicament for treating severe asthma in a subject.

The present disclosure also provides a method of treating severe asthma in a subject, the method comprising administering to the subject a compound that binds to or specifically binds to CD131 and neutralises signalling by interleukin (IL) 5 and granulocyte-macrophage colony stimulating factor (GM-CSF) in a subject.

The present disclosure also provides a compound that binds to or specifically binds to CD131 and neutralises signalling by IL-5 and GM-CSF for use in treating severe asthma in a subject. The present disclosure also provides use of a compound that binds to or specifically binds to CD131 and neutralises signalling by IL-5 and GM-CSF in the manufacture of a medicament for treating severe asthma in a subject.

The findings by the inventors also provide the basis for methods of depleting and/or reducing eosinophils and/or neutrophils in a subject suffering from severe asthma, the method comprising administering to the subject one or more compound(s) that neutralize signaling by granulocyte-macrophage colony stimulating factor (GM-CSF) and interleukin (IL) 5 to the subject. The present disclosure also provides one or more compound(s) that neutralize signaling by GM-CSF and IL-5 for use in depleting and/or reducing eosinophils and/or neutrophils in a subject suffering from severe asthma. The present disclosure further provides use of one or more compound(s) that neutralize signaling by GM-CSF and IL-5 in the manufacture of a medicament for depleting and/or reducing eosinophils and/or neutrophils in a subject suffering from severe asthma.

The findings by the inventors also provide the basis for methods of depleting and/or reducing eosinophils and/or neutrophils in a subject suffering from severe asthma, the method comprising administering to the subject a compound that binds to or specifically binds to CD131 and neutralises signalling by interleukin (IL) 5 and granulocyte-macrophage colony stimulating factor (GM-CSF) in a subject.

In one example, the disclosure provides a method of depleting and/or reducing eosinophils in a subject suffering from severe asthma. For example, the disclosure provides a method of depleting eosinophils in a subject suffering from severe asthma. In another example, the disclosure provides a method of reducing eosinophils in a subject suffering from severe asthma. In a further example, the disclosure provides a method of depleting and reducing eosinophils in a subject suffering from severe asthma.

In one example, the disclosure provides a method of depleting and/or reducing neutrophils in a subject suffering from severe asthma. For example, the disclosure provides a method of depleting neutrophils in a subject suffering from severe asthma. In another example, the disclosure provides a method of reducing neutrophils in a subject suffering from severe asthma. In a further example, the disclosure provides a method of depleting and reducing neutrophils in a subject suffering from severe asthma.

In one example, the disclosure provides a method of depleting and/or reducing eosinophils and neutrophils in a subject suffering from severe asthma. For example, the disclosure provides a method of depleting eosinophils and neutrophils in a subject suffering from severe asthma. In another example, the disclosure provides a method of reducing eosinophils and neutrophils in a subject suffering from severe asthma. In a further example, the disclosure provides a method of depleting and reducing eosinophils and neutrophils in a subject suffering from severe asthma.

The present disclosure also provides a compound that binds to or specifically binds to CD131 and neutralises signalling by IL-5 and GM-CSF for use in depleting and/or reducing eosinophils and/or neutrophils in a subject suffering from severe asthma. The present disclosure further provides a compound that binds to or specifically binds to CD131 and neutralises signalling by IL-5 and GM-CSF in the manufacture of a medicament for depleting and/or reducing eosinophils and/or neutrophils in a subject suffering from severe asthma.

The present disclosure also provides a method of treating eosinophilia and/or neutrophilia in a subject suffering from severe asthma, the method comprising administering to the subject a compound that binds to or specifically binds to CD131 and neutralises signalling by IL-5 and GM-CSF in a subject. In one example, the disclosure provides a method of treating eosinophilia in a subject suffering from severe asthma. In another example, the disclosure provides a method of treating neutrophilia in a subject suffering from severe asthma. In a further example, the disclosure provides a method of treating eosinophilia and neutrophilia in a subject suffering from severe asthma.

The present disclosure also provides a compound that binds to or specifically binds to CD131 and neutralises signalling by IL-5 and GM-CSF for use in treating eosinophilia and/or neutrophilia in a subject suffering from severe asthma. The present disclosure further provides a compound that binds to or specifically binds to CD131 and neutralises signalling by IL-5 and GM-CSF in the manufacture of a medicament for treating eosinophilia and/or neutrophilia in a subject suffering from severe asthma.

In one example, the subject is suffering from severe asthma (i.e., the subject is in need of treatment). For example, the subject is suffering from clinically diagnosed severe asthma.

Methods of clinically diagnosing severe asthma will be apparent to the skilled person and/or are described herein. In one example, the subject satisfies the European Respiratory Society (ERS) and the American Thoracic Society (ATS) definition of severe asthma. Thus, in some examples, the subject:
(i) has, or is receiving treatment, with:
   a) high dose inhaled corticosteroid plus at least one additional controller; or
   b) oral corticosteroids for >6 months/year; and
(ii) has at least one or more, or all, of the following:
   a) asthma control test (ACT)<20 or asthma control questionnaire (ACQ)>1.5;
   b) at least 2 exacerbations in the last 12 months;
   c) at least 1 exacerbation treated in hospital or requiring mechanical ventilation in the last 12 months; and
   d) forced expiratory volume in one second (FEV1)<80% if the FEV₁/FVC (forced vital capacity) is below the lower limit of normal.

In one example, the subject has asthma that is uncontrolled despite non-biologic treatment, e.g., the subject has previously received treatment with a small molecule drug. For example, the subject has asthma and has failed all non-biologic treatments.

In one example, the subject has asthma that is uncontrolled despite all treatments, including biologic treatment.

Biologic treatments used for the treatment of severe asthma will be apparent to the skilled person and/or described herein. For example, the biologic treatment comprises an anti-IgE antibody (e.g., omalizumab), an anti-IL5 antibody (e.g., mepolizumab or reslizumab or benralizumab), an IL-13 antibody (e.g., dupilumab), an anti-1L-17A antibody, (e.g., secukinumab) and/or an anti-thymic stromal lymphopoietin (TSLP) antibody, e.g., tezepelumab.

Non-biologic treatments used for the treatment of severe asthma will be apparent to the skilled person and/or described herein. For example, the non-biologic treatment comprises an immunomodulator or an immunosuppressant, a corticosteroid (e.g. beclometasone (e.g., Qvar®, Beconase AQ®, budesonide (e.g., Pulmicort Flexhaler®), ciclesonide (e.g., Alvesco®), or fluticasone (e.g., Flovent® HFA)), a 32 agonist (e.g., salbutamol (e.g., Ventolin®), terbutaline sulfate (e.g., Bricanyl®, Marex®), formoterol (e.g., Perforomist®), or salmeterol (e.g., Serevent®)), a leukotriene receptor antagonist (e.g., montelukast (e.g., Singulair®)), a muscarinic antagonist (e.g., ipratropium bromide (e.g., Atrovent®)), a theophylline (e.g., aminophylline (e.g., Euphyllin®)), magnesium sulfate, and a mast cell stabilizer (e.g., sodium cromoglycate or nedocromil (e.g., Tilade®)).

In one example, the subject satisfies the Global Initiative for Asthma (GINA) definition of severe asthma. Thus, in some examples, the subject has asthma that is uncontrolled despite high dose inhaled corticosteroids (ICS)-long-acting beta2-agonist (LABA), or that requires high dose ICS-LABA to remain controlled.

In one example, the subject has, or is receiving treatment with (i) high dose inhaled corticosteroid plus at least one additional controller; or oral corticosteroids for >6 months/year; and (ii) at least 2 exacerbations in the last 12 months.

In one example, the severe asthma is treatment resistant or refractory severe asthma and/or severe uncontrolled asthma.

In one example, the severe asthma is treatment resistant or refractory severe asthma. For example, the severe asthma corticosteroid-refractory severe asthma, corticosteroid-intolerant severe asthma, corticosteroid-resistant severe asthma or corticosteroid-dependent severe asthma. In one example, the severe asthma is corticosteroid-refractory severe asthma. In one example, the severe asthma is corticosteroid-intolerant severe asthma. In one example, the severe asthma is corticosteroid-resistant severe asthma. In one example, the severe asthma is corticosteroid-dependent severe asthma.

In one example, the severe asthma is severe uncontrolled asthma.

The present disclosure also provides a method of treating severe uncontrolled asthma in a subject, regardless of eosinophilic phenotype, wherein the subject has had at least two asthma exacerbations during the previous 12 months, the method comprising administering to the subject a compound that binds to or specifically binds to CD131 and neutralises signalling by interleukin (IL) 5 and granulocyte-macrophage colony stimulating factor (GM-CSF) in a subject.

In one example, the compound that binds to or specifically binds to CD131 and neutralises signalling by interleukin (IL) 5 and granulocyte-macrophage colony stimulating factor (GM-CSF) is administered as a first line therapy. For example, the compound is administered as a first line therapy in a subject that has failed all non-biologic treatment options.

In one example, compound that binds to or specifically binds to CD131 and neutralises signalling by interleukin (IL) 5 and granulocyte-macrophage colony stimulating factor (GM-CSF) is administered to a subject that has failed all treatment options. For example, the subject has failed all biologic and non-biologic treatment options.

In one example, the severe asthma is eosinophilic asthma, neutrophilic asthma, paucigranulocytic asthma or mixed granulocytic asthma. For example, the severe asthma is eosinophilic asthma. In another example, the severe asthma is neutrophilic asthma. In one example, the severe asthma is paucigranulocytic asthma. In another example, the severe asthma is mixed granulocytic asthma.

In one example, the severe asthma is a T_{H}2-high asthma or a T_{H}2-low asthma. For example, the severe asthma is a T_{H}2-high asthma. In one example, the T_{H}2-high asthma is eosinophilic asthma. In one example, the severe asthma is a T_{H}2-low asthma. For example, the T_{H}2-low asthma is neutrophilic, paucigranulocytic asthma or mixed granulocytic asthma. In one example, the T_{H}2-low asthma is neutrophilic asthma. In another example, the T_{H}2-low asthma is mixed granulocytic asthma. In a further example, the T_{H}2-low asthma is paucigranulocytic asthma.

In one example, the compound that binds to CD131 is administered as an add-on maintenance therapy. For example, the compound that binds to CD131 is administered as an add-on maintenance therapy to a standard of care therapy. In one example, the compound that binds to CD131 is administered as an add-on maintenance therapy, wherein the subject is 12 years and older with severe uncontrolled asthma.

In one example, the compound that binds to CD131 is administered as a first line maintenance therapy to the subject as an add-on to a standard of care therapy. For example, the compound that binds to CD131 is administered as a first line maintenance therapy to the subject as an add-on to a standard of care therapy comprising inhaled corticosteroids, long-acting β-agonist and tiotropium, wherein the subject has severe uncontrolled asthma regardless of inflammatory phenotype and biomarker threshold.

In one example, the subject has, or is suffering from, severe asthma regardless of the eosinophilic phenotype, wherein the severe asthma is uncontrolled despite maximum standard of care therapy, and wherein the subject has had at least 2 asthma exacerbations during the previous 12 months.

In one example, the subject is receiving a standard of care therapy at the time of administering the compound that binds to CD131. For example, the subject is receiving an additional therapy at the time of administering the compound that binds to CD131.

In one example, the compound that binds to CD131 is administered in combination with a standard of care therapy. For example, the compound that binds to CD131 is administered in combination with an additional therapy.

In one example, the subject is not receiving a standard of care therapy at the time of administering the compound that binds to CD131. For example, the subject is not receiving an additional therapy at the time of administering the compound that binds to CD131.

In one example, the additional therapy (or standard of care therapy) comprises an anti-inflammatory compound, and immuno-modulator, an immunosuppressant, a corticosteroid, a β2 agonist, leukotriene receptor antagonist, muscarinic antagonist, theophylline, magnesium sulfate, anti-thymic stromal lymphopoietin (TSLP) antibody, tiotropium, anti-IL-5 antibody, anti-IL-13 antibody and/or anti-1L-17A antibody. In one example, the additional therapy is an anti-inflammatory compound. In one example, the additional therapy is an immunomodulator or an immunosuppressant. In one example, the additional therapy is a corticosteroid, e.g. a glucocorticoid. For example, the corticosteroid is an inhaled corticosteroid or an oral corticosteroid. In one example, the additional therapy is a β2 agonist. In one example, the additional therapy is a leukotriene receptor antagonist. In one example, the additional therapy is a muscarinic antagonist. In one example, the additional therapy is a theophylline. In one example, the additional therapy is magnesium sulfate. In one example, the additional therapy is a mast cell stabilizer. In one example, the additional therapy is an anti-IL-5 antibody, e.g., mepolizumab or reslizumab or benralizumab. In one example, the additional therapy is an anti-1L-13 antibody, e.g., dupilumab. In one example, the additional therapy is an anti-1L-17A antibody, e.g., secukinumab. In one example, the additional therapy is an anti-thymic stromal lymphopoietin (TSLP) antibody, e.g., tezepelumab. In one example, the additional therapy is tiotropium. In one example, the additional therapy is one or more inhaled corticosteroids, one or more oral corticosteroids, one or more long-acting β-agonists and/or tiotropium. In one example, the additional therapy comprises inhaled corticosteroids, a long-acting β-agonist and tiotropium.

In one example, the subject has, or suffers from, asthma-chronic obstructive pulmonary disease (COPD) overlap (ACO), airway remodelling, fibrosis and/or persistent airflow limitation. For example, the subject has, or suffers from, asthma-chronic obstructive pulmonary disease (COPD) overlap (ACO). In another example, the subject has, or suffers from, airway remodelling. In a further example, the subject has, or suffers from, fibrosis. In one example, the subject has, or suffers from, persistent airflow limitation. In one example, persistent airflow limitation (or persistent airway obstruction) is defined by a forced expiratory volume in one second (FEV₁) of <80% predicted with a diurnal peak expiratory flow (PEF) variability of >20%.

In one example, the compound that binds to CD131 is administered in an amount sufficient to reduce or prevent an increase in mucus production. Increased production of mucus in the respiratory tract is a common effect of asthma, which leads to breathing difficulties and coughing. Furthermore, airway mucus plugging is frequently associated with death that results from asthma. Secretion of mucus from goblet cells also contributes to airway hyperresponsiveness (AHR) in concert with airway smooth muscle contraction. Levels of mucus production can be estimated based on the volume and frequency of sputum produced by a subject. Levels of mucus production can also be assessed using histology techniques which are routine in the art, for example by staining with Alcian blue-periodic acid Schiff (AB-PAS) stain. Mucus production in the airways is also associated with expression of the mucin gene Muc5ac. Thus, mucus production can also be assessed by measuring Muc5ac expression by RT-PCR performed on lung tissue.

In one example, the compound that binds to CD131 is administered in an amount sufficient to reduce the severity of or prevent onset of one or more symptoms of severe asthma.

In one example, administration of the compound that binds to CD131 reduces, or prevents an increase in, one or more or all of the following:
(i) amount of monocytes in the subject's blood;
(ii) amount of neutrophils in the subject's blood;
(iii) total amount of cells in the bronchoalveolar (BAL) fluid of the subject's lung;
(iv) macrophage accumulation in the BAL fluid of the subject's lung;
(v) neutrophil accumulation in BAL fluid of the subject's lung;
(vi) eosinophil accumulation in the BAL fluid of the subject's lung;
(vii) leukocyte accumulation in the subject's lung;
(viii) neutrophil accumulation in the subject's lung;
(ix) eosinophil accumulation in the subject's lung;
(x) interstitial macrophage accumulation in the subject's lung;
(xi) alveolar macrophage accumulation in the subject's lung;
(xii) airway fibrosis of the subject's lung; and/or
(xiii) inflammation of the subject's lung.

In some examples, administration of the compound that binds to CD131 reduces, or prevents an increase in macrophage accumulation. For example, administration of the compound that binds to CD131 reduces, or prevents an increase in macrophage accumulation in the BAL fluid of the subject's lung. In another example, administration of the compound that binds to CD131 reduces, or prevents an increase in macrophage accumulation in the subject's lung. For example, the macrophages are alveolar macrophages, monocyte derived interstitial macrophages and/or blood monocytes.

In one example, the macrophages are alveolar macrophages. In one example, administration of the compound that binds to CD131 reduces, or prevents an increase in, alveolar macrophage accumulation in the subject's lung. For example, the alveolar macrophages are CD45⁺F4/80⁺CD11c^{high}SiglecF^{high} macrophages.

In one example, the macrophages are monocyte derived interstitial macrophages. In one example, administration of the compound that binds to CD131 reduces, or prevents an increase in, interstitial macrophage accumulation in the subject's lung. For example, the macrophages are CD11b+ monocyte derived interstitial macrophages. In one example, the macrophages are CD45⁺F4/80⁺CD11c⁺CD11b⁺ macrophages.

In one example, the macrophages are monocytes. For example, the monocytes are blood monocytes. In one example, administration of the compound that binds to CD131 reduces, or prevents an increase in the amount of monocytes in the subject's blood. In one example, the monocytes are CD45⁺F4/80⁺CD11c⁻CD11b⁺ monocytes.

In some examples, administration of the compound that binds to CD131 reduces or prevents an increase in the amount of neutrophils in the subject's blood. In another example, administration of the compound that binds to CD131 reduces or prevents an increase in neutrophil accumulation in BAL fluid of the subject's lung. In a further example, administration of the compound that binds to CD131 reduces or prevents an increase in neutrophil accumulation in the subject's lung. In one example, the neutrophils are CD45⁺Ly6G^{high}CD1b⁺ neutrophils.

In some examples, administration of the compound that binds to CD131 reduces, or prevents an increase in eosinophil accumulation in the subject's lung. In another example, administration of the compound that binds to CD131 reduces, or prevents an increase in eosinophil accumulation in the BAL fluid of the subject's lung. In one example, the eosinophils are CD45⁺F4/80⁺CD11c^{low}SiglecF^{high} eosinophils.

In some examples, administration of the compound that binds to CD131 reduces, or prevents an increase in, leukocyte accumulation in the subject's lung. For example, the leukocytes are CD45⁺ leukocytes.

In some examples the compound that binds to CD131 is administered in an amount sufficient to reduce, or prevent an increase in, total cell counts and/or total protein in BALF of the subject. In some examples, the compound that binds to CD131 is administered in an amount sufficient to reduce, or prevent an increase in, the level of neutrophils present in BALF of the subject. In another example, the compound that binds to CD131 is administered in an amount sufficient to reduce, or prevent an increase in, the total amount of cells in the bronchoalveolar (BAL) fluid of the subject's lung.

In some examples, the compound that binds to CD131 is administered in an amount sufficient to reduce, or prevent an increase in, the level of neutrophil elastase and/or myeloperoxidase activity in BALF of the subject.

In one example, the compound that binds to CD131 is administered in an amount sufficient to reduce, or prevent an increase in, airway fibrosis of the subject's lung.

In one example, the compound that binds to CD131 is administered in an amount sufficient to reduce, or prevent an increase in, inflammation in the subject's lungs. In one example, the inflammation is alveolar and/or peribronchiolar inflammation. In one example, the inflammation is alveolar inflammation. In another example, the inflammation is peribronchiolar inflammation. In a further example, the inflammation is alveolar and peribronchiolar inflammation.

Methods for assessing the above will be known in the art. For example, the levels of neutrophils, eosinophils, and other cells in lung tissue or BAL fluid can be measured by flow cytometry or immunohistochemistry (e.g., as described in Wang et al., Clin Sci Lond, 2017 131:2347-2362).

In one example, administration of the compound that binds to CD131 reduces, or prevents an increase in, one or more or all of the following:
(i) airway hyper-responsiveness (AHR);
(ii) baseline airway restriction;
(iii) total respiratory resistance of the subject;
(iv) airway resistance of the subject;
(v) airspace enlargement in the subject
(vi) collagen fibre deposition in the subject's lung;
(vii) amount of BALF protein in the BAL fluid of the subject's lung;
(viii) lactate dehydrogenase (LDH) levels in the BAL fluid of the subject's lung;
(ix) NETosis in the subject's lung;
(x) myeloperoxidase activity in the BAL fluid of the subject's lung; and/or
(xi) amount of double stranded DNA (dsDNA) in the BAL fluid of the subject's lung.

In one example, the compound that binds to CD131 is administered in an amount sufficient to reduce, or prevent an increase in, airway hyper-responsiveness (AHR). AHR is an increased sensitivity of the airways to an inhaled constrictor agonist, a steeper slope of the dose-response curve, and a greater maximal response to the agonist. AHR is generally associated with lower lung function and asthmatic symptoms. AHR can be assessed, for example, with a bronchial challenge test. This most often uses constrictor agonists like methacholine or histamine. These chemicals trigger bronchospasm in non-asthmatic subjects as well, but subjects with AHR have a lower response threshold to the constrictor agonists. In one example, airway hyper-responsiveness includes measuring total respiratory system resistance in the subject, airway resistance in the subject, respiratory compliance in the subject, respiratory system elastance and/or baseline airway restriction in the subject. Methods of measuring and/or determining airway hyper-responsiveness in a subject will be apparent to the skilled person and/or described herein. Exemplary methods are described in (FitzPatrick et al., Sci Rep, 2016 6:22751).

In one example, the compound that binds to CD131 is administered in an amount sufficient to reduce, or prevent an increase in, collagen fibre deposition in the subject's lung. Methods of determining collagen fibre deposition will be apparent to the skilled person and include, for example, Masson's Trichrome staining.

In one example, the compound that binds to CD131 is administered in an amount sufficient to reduce, or prevent an increase in, the amount of BALF protein in the BAL fluid of the subject's lung.

In one example, the compound that binds to CD131 is administered in an amount sufficient to reduce, or prevent an increase in, lactate dehydrogenase (LDH) levels in the BAL fluid of the subject's lung.

In one example, the compound that binds to CD131 is administered in an amount sufficient to reduce, or prevent an increase in, the formation of neutrophil extracellular traps (NETs), or “NETosis”. Methods for measuring neutrophilic lung inflammation are known in the art and include detecting the quantity of neutrophils in lung tissue or bronchoalveolar lavage by flow cytometry or immunohistochemistry (e.g., as described in Wang et al., Clin Sci Lond, 2017 131:2347-2362). Neutrophilic inflammation can also be assessed by detecting NETosis markers. For example, suitable methods include measuring neutrophil elastase activity on BAL fluid, for example by using an EnzChek™ Elastase Assay Kit. In addition, NETosis markers include myeloperoxidase (MPO) activity and/or dsDNA.

In one example, the compound that binds to CD131 is administered in an amount sufficient to reduce, or prevent an increase in, myeloperoxidase (MPO) activity in the BAL fluid of the subject's lung.

In one example, the compound that binds to CD131 is administered in an amount sufficient to reduce, or prevent an increase in, the amount of double stranded DNA (dsDNA) in the BAL fluid of the subject's lung. Content of extracellular double stranded DNA (dsDNA) in the BAL fluid, which is characteristic of neutrophilic inflammation, can be measured using, for example, Quant-iT PicoGreen dsDNA reagent.

In one example, the compound that binds to CD131 is administered in an amount sufficient to enhance lung function. Methods of determining lung function will be apparent to the skilled person and include, for example, spirometry.

In one example, the compound that binds to CD131 is administered in an amount sufficient to:
(i) reduce asthma exacerbation rates (AER) in the subject by at least 60% (e.g., compared to administration of placebo in the overall population);
(ii) prolong onset to the first exacerbation in the subject;
(iii) improve lung function in the subject as measured by trough forced expiratory volume in one second (FEV₁); and/or
(iv) safely reduce or safely curtail corticosteroid use in the subject.

In one example, the compound that binds to CD131 is administered in an amount sufficient to reduce the severity of and/or the frequency of and/or prevent or delay onset of exacerbations. For example, the compound that binds to CD131 is administered in an amount sufficient to reduce the severity of or the frequency of or prevent onset of acute exacerbations. For example, the compound that binds to CD131 is administered in an amount sufficient to reduce the severity of asthma exacerbations. In another example, the compound that binds to CD131 is administered in an amount sufficient to reduce the frequency of asthma exacerbations. In a further example, the compound that binds to CD131 is administered in an amount sufficient to prevent or delay onset of asthma exacerbations. In one example, the compound that binds to CD131 is administered in an amount sufficient to prolong onset to the first exacerbation in the subject.

In one example, the compound that binds to CD131 is administered in an amount sufficient to reduce the asthma exacerbation rate (AER) by at least 60% (e.g., compared to a subject to which the compound was not administered). In one example, the compound that binds to CD131 is administered in an amount sufficient to reduce the asthma exacerbation rate (AER) by at least 60% (e.g., compared to administration of placebo in the overall population). For example, the AER is reduced by at least 60%, or at least 65%, or at least 70%, or at least 75%, or at least 80%, or at least 85%, or at least 90%, or at least 95%. In one example, the compound that binds to CD131 is administered in an amount sufficient to reduce the asthma exacerbation rate (AER) by at least 65% (e.g., compared to a subject to which the compound was not administered). In one example, the compound that binds to CD131 is administered in an amount sufficient to reduce the asthma exacerbation rate (AER) by at least 65% (e.g., compared to administration of placebo in the overall population). In one example, the compound that binds to CD131 is administered in an amount sufficient to reduce the asthma exacerbation rate (AER) by at least 70% (e.g., compared to a subject to which the compound was not administered). In one example, the compound that binds to CD131 is administered in an amount sufficient to reduce the asthma exacerbation rate (AER) by at least 70% (e.g., compared to administration of placebo in the overall population). In one example, the compound that binds to CD131 is administered in an amount sufficient to reduce the asthma exacerbation rate (AER) by at least 75% (e.g., compared to a subject to which the compound was not administered). In one example, the compound that binds to CD131 is administered in an amount sufficient to reduce the asthma exacerbation rate (AER) by at least 75% (e.g., compared to administration of placebo in the overall population). In one example, the compound that binds to CD131 is administered in an amount sufficient to reduce the asthma exacerbation rate (AER) by at least 80% (e.g., compared to a subject to which the compound was not administered). In one example, the compound that binds to CD131 is administered in an amount sufficient to reduce the asthma exacerbation rate (AER) by at least 80% (e.g., compared to administration of placebo in the overall population). In one example, the compound that binds to CD131 is administered in an amount sufficient to reduce the asthma exacerbation rate (AER) by at least 85% (e.g., compared to a subject to which the compound was not administered). In one example, the compound that binds to CD131 is administered in an amount sufficient to reduce the asthma exacerbation rate (AER) by at least 85% (e.g., compared to administration of placebo in the overall population). In one example, the compound that binds to CD131 is administered in an amount sufficient to reduce the asthma exacerbation rate (AER) by at least 90% (e.g., compared to a subject to which the compound was not administered). In one example, the compound that binds to CD131 is administered in an amount sufficient to reduce the asthma exacerbation rate (AER) by at least 90% (e.g., compared to administration of placebo in the overall population). In one example, the compound that binds to CD131 is administered in an amount sufficient to reduce the asthma exacerbation rate (AER) by at least 95% (e.g., compared to a subject to which the compound was not administered). In one example, the compound that binds to CD131 is administered in an amount sufficient to reduce the asthma exacerbation rate (AER) by at least 95% (e.g., compared to administration of placebo in the overall population).

In one example, the compound that binds to CD131 is administered in an amount sufficient to increase FEV₁, e.g., compared to the FEV₁ in the subject before the compound was administered. In one example, the compound that binds to CD131 is administered in an amount sufficient to improve lung function in the subject as measured by trough forced expiratory volume in one second (FEV₁). In another example, the compound that binds to CD131 is administered in an amount sufficient to increase FVC (forced vital capacity), e.g., compared to the FVC in the subject before the compound was administered.

In one example, the compound that binds to CD131 is administered in an amount sufficient to safely reduce or safely curtail corticosteroid use in the subject.

In one example, administration of the compound that binds to CD131 reduces, or prevents an increase in the level of one or more or all of the following: pro-type I collagen (Col1a1), beta chain cytokine receptor (CSF2RB), Interleukin 13 (IL-13), IL-4, IL-17, IL-12b, interferon gamma (Ifng), C—C Motif Chemokine Ligand 17 (CCL17), Ccl1, matrix metallopeptidase 12 (Mmp12), Mmp13, Thrombospondin 4 (Thbs4), transforming growth factor-beta (Tgfb1), Proteoglycan 2 (Prg2), Cd3d, Cd8a, Cd4, Cd79a, Cd86, chitinase-like 3 (Chil3), arginase 1 (Arg1), eosinophil-associated, ribonuclease A family member 2 (Ear2), histocompatibility 2, class II antigen A, beta 1 (H2-Ab1) and fibromodulin (Fmod).

In one example, administration of the compound that binds to CD131 reduces, or prevents an increase in the level of one or more or all of the following: pro-type I collagen (Col1a1), beta chain cytokine receptor (CSF2RB), Interleukin 13 (IL-13), IL-4, IL-17, IL-12b, interferon gamma (Ifng) and C—C Motif Chemokine Ligand 17 (CCL17).

In one example, administration of the compound that binds to CD131 reduces, or prevents an increase in the level of one or more or all of the following genes involved in inflammation and immunity: Cd3d, Cd8a, Cd4, Cd79a, Chil3, Arg1, Ear2, Prg2, Cd86, H2-Ab1, Ccl11 and Ccl17.

In one example, administration of the compound that binds to CD131 reduces, or prevents an increase in the level of one or more or all of the following genes involved in tissue remodelling: Mmp12, Mmp13, Tgfb1, Thbs4 and Fmod.

In one example, administration of the compound that binds to CD131 reduces, or prevents an increase in the level of expression of one or more genes in the subject's lung selected from the group consisting of pro-type I collagen (Col1a1), beta chain cytokine receptor (CSF2RB), Interleukin 13 (IL-13), IL-4, IL-17, IL-12b, interferon gamma (Ifng), C—C Motif Chemokine Ligand 17 (CCL17), Ccl11, matrix metallopeptidase 12 (Mmp12), Mmp13, Thrombospondin 4 (Thbs4), transforming growth factor-beta (Tgfb1), Proteoglycan 2 (Prg2), Cd3d, Cd8a, Cd4, Cd79a, Cd86, chitinase-like 3 (Chil3), arginase 1 (Arg1), eosinophil-associated, ribonuclease A family member 2 (Ear2), histocompatibility 2, class II antigen A, beta 1 (H2-Ab1) and fibromodulin (Fmod).

In one example, administration of the compound that binds to CD131 reduces, or prevents an increase in the level of expression of one or more genes in the subject's lung selected from the group consisting of pro-type I collagen (Col1a1), beta chain cytokine receptor (CSF2RB), Interleukin 13 (IL-13), IL-4, IL-17, IL-12b, interferon gamma (Ifng) and C—C Motif Chemokine Ligand 17 (CCL17).

In one example, administration of the compound that binds to CD131 reduces, or prevents an increase in the level of expression of one or more genes in the subject's lung selected from the group consisting of Cd3d, Cd8a, Cd4, Cd79a, Chil3, Arg1, Ear2, Prg2, Cd86, H2-Ab, Ccl11 and Ccl17.

In one example, administration of the compound that binds to CD131 reduces, or prevents an increase in the level of expression of one or more genes in the subject's lung selected from the group consisting of Mmp12, Mmp13, Tgfb1, Thbs4 and Fmod.

In one example, the compound that binds to CD131 is administered in an amount sufficient to reduce, or prevent an increase in, the level of chemokine gene expression in the subject. For example, the compound that binds to CD131 is administered in an amount sufficient to reduce, or prevent an increase in, the level of expression of the chemokine CCL17 in the subject.

In one example, the compound that binds to CD131 is administered in an amount sufficient to reduce, or prevent an increase in, the level of expression of inflammatory cytokine genes in the subject. For example, the compound binds to CD131 is administered in an amount sufficient to reduce, or prevent an increase in, the level of expression of the inflammatory cytokines Interleukin 13 (IL-13), IL-4, IL-17, IL-12b, interferon gamma (Ifng) in the subject. In one example, the compound binds to CD131 is administered in an amount sufficient to reduce, or prevent an increase in, the level of expression of the Th2/Th17 inflammatory cytokines Interleukin 13 (IL-13), IL-4 and IL-17 in the subject. In one example, the compound binds to CD131 is administered in an amount sufficient to reduce, or prevent an increase in, the level of expression of the Th1 inflammatory cytokines IL-12b and interferon gamma (Ifng) in the subject.

In one example, administration of the compound that binds to CD131 reduces, or prevents an increase in the level of one or more or all of the following: phosphorylated STAT5 (pSTAT5), CD35 and CD11b.

In one example, administration of the compound that binds to CD131 increases, or prevents a decrease in the level of CD62L or CD63. For example, administration of the compound that binds to CD131 increases, or prevents a decrease in the level of CD62L.

Methods of measuring the level of gene expression in the subject's lung and/or blood cells will be apparent to the skilled person and/or described herein. For example, the level of gene expression is measured using real-time quantitative PCR, wherein the level of gene expression is normalised against a housekeeping gene (e.g., GAPDH).

In some examples, the levels of the above genes and/or proteins are reduced, or prevented from increasing, in the subject's lungs. In some examples, the levels of expression of the above genes are reduced, or prevented from increasing, in the subject's blood cells.

In one example, the levels of the above genes and/or proteins are reduced, or prevented from increasing, in a sample obtained from the subject. For example, the sample is a tissue and/or a fluid sample. For example, the tissue is a lung tissue sample, such as a lung biopsy sample. For example, the fluid sample is a blood sample (e.g., a whole blood sample or a serum sample), a bronchoalveolar (BAL) fluid sample or a sputum sample. It will be apparent to the skilled person that the fluid sample contains or comprises cells (e.g., inflammatory cells, such as eosinophils).

In one example of any method described herein, the compound that binds to CD131 and neutralizes signaling by GM-CSF and IL-5 also neutralizes signaling by IL-3.

In one example, the compound that binds to CD131 of the disclosure competes with IL-3 and/or GM-CSF and/or IL-5 for binding to a cell expressing CD131 (e.g., TF-1 cells).

In one example, the compound that binds to CD131 inhibits:
(a) GM-CSF-induced proliferation of TF-1 erythroleukemic cells with an IC₅₀ of at least 460 nM; and/or
(b) IL-5-induced proliferation of TF-1 erythroleukemic cells with an IC₅₀ of at least 1600 nM.

In one example, the compound that binds to CD131 additionally inhibits IL-3-induced proliferation of TF-1 erythroleukemic cells with an IC₅₀ of at least 10 nM.

In one example, the compound that binds to CD131 inhibits:
(a) GM-CSF-induced proliferation of TF-1 erythroleukemic cells with an IC₅₀ of at least 460 nM; and/or
(b) IL-5-induced proliferation of TF-1 erythroleukemic cells with an IC₅₀ of at least 1600 nM; and/or
(c) IL-3-induced proliferation of TF-1 erythroleukemic cells with an IC₅₀ of at least 10 nM.

In one example, the compound that binds to CD131 inhibits GM-CSF-induced proliferation of TF-1 cells with an IC₅₀ of at least about 460 nM. For example, the IC₅₀ is at least about 300 nM or 200 nM or 100 nM. For example, the IC₅₀ is at least about 460 nM. For example, the IC₅₀ is at least about 10 nM or 5 nM or 1 nM. In one example, the IC₅₀ is at least about 1 nM. For example, the IC₅₀ is at least about 0.9 nM or 0.8 nM or 0.6 nM. In one example, the IC₅₀ is at least about 0.5 nM. In one example, the IC₅₀ is at least about 0.4 nM. In one example, the IC₅₀ is at least about 0.3 nM.

In one example, the compound that binds to CD131 inhibits IL-3-induced proliferation of TF-1 cells with an IC₅₀ of at least about 10 nM. For example, the IC₅₀ is at least about 10 nM or 5 nM or 1 nM. In one example, the IC₅₀ is at least about 1 nM. For example, the IC₅₀ is at least about 0.9 nM or 0.8 nM or 0.6 nM. In one example, the IC₅₀ is at least about 0.5 nM. In one example, the IC₅₀ is at least about 0.2 nM or at least about 0.1 nM. In one example, the IC₅₀ is at least about 0.15 nM.

In one example, the compound that binds to CD131 inhibits IL-5-induced proliferation of TF-1 cells with an IC₅₀ of at least 1600 nM. For example, the IC₅₀ is at least about 460 nM. For example, the IC₅₀ is at least about 1500 nM or about 1000 nM or about 500 nM. For example, the IC₅₀ is at least about 460 nM. For example, the IC₅₀ is at least about 300 nM or 200 nM or 100 nM. For example, the IC₅₀ is at least about 10 nM or nM or 1 nM. In one example, the IC₅₀ is at least about 5 nM. For example, the IC₅₀ is at least about 4 nM. In one example, the IC₅₀ is at least about 4.5 nM or at least about 4.6 or at least about 4.7 nM. In one example, the IC₅₀ is at least about 4.6 nM.

Methods for determining the IC₅₀ are described herein and include culturing TF-1 cells (e.g., about 1×10⁴ TF-1 cells) in the presence of the CD131-binding protein (e.g., for at least about 3 minutes or 1 hour, such as about 30 minutes) prior to adding the relevant growth factor (GM-CSF, IL-3 and/or IL-5) and culturing the cells further (e.g., for at least about 48 hours or at least about 72 hours or at least about 96 hours, e.g., for about 72 hours) and then determining cell proliferation. Cell proliferation can be determined by growing the cells in the presence of ³[H]-thymidine for about 6 hours and determining ³[H]-thymidine incorporation, e.g., by liquid-scintillation counting. By determining proliferation in a variety of concentrations of the CD131-binding protein an IC₅₀ can be determined.

In one example, the compound that binds to CD131 is a CD131-binding protein. For example, a CD131-binding protein comprising an antigen binding domain of an antibody, wherein the antigen binding domain binds to or specifically binds to CD131 and neutralizes signalling by IL-5 and GM-CSF. In some examples, the antigen binding site comprises one or more complementarity determining regions (CDRs).

Reference herein to a compound or protein or antibody that “binds to” CD131 provides literal support for a compound or protein or antibody that “binds specifically to” or “specifically binds to” CD131.

In one example, the CD131-binding protein binds to a polypeptide comprising a sequence set forth in SEQ ID NO: 192 with a K_{D} of about 100 nM or less, e.g., when the polypeptide is immobilized on a solid surface and the K_{D} is determined by surface plasmon resonance. In one example, the K_{D} is 10 nM or less, for example, 5 nM or less or 4 nM or less, or 3 nM or less or 2 nM or less. In one example, the K_{D} is 1 nM or less. In one example, the K_{D} is 0.9 nM or less or 0.7 nM or less or 0.8 nM or less or 0.7 nM or less or 0.6 nM or less. In one example, the K_{D} is 0.5 nM or less. In one example, the K_{D} is 0.4 nM or less. In one example, the K_{D} is 0.3 nM or less.

In one example, the CD131-binding protein binds to a cell expressing CD131 (e.g., a neutrophil or an eosinophil or a TF-1 cell) with a K_{D} of about 10 nM or less, e.g., using a competition assay using labeled and unlabeled protein or antibody. In one example, the K_{D} is 5 nM or less or 4 nM or less, or 3 nM or less or 2 nM or less. In one example, the K_{D} is 1 nM or less. In one example, the K_{D} is 0.9 nM or less or 0.7 nM or less or 0.8 nM or less or 0.7 nM or less or 0.6 nM or less.

In one example, the K_{D} is about 300 nM or less for a neutrophil.

In one example, the K_{D} is about 700 nM or less for an eosinophil.

In one example, the K_{D} is about 400 nM or less for a TF-1 cell.

In one example, the compound is a CD131-binding protein comprising an antigen binding site that:
(a) binds to or specifically binds to an epitope within site 2 of CD131 and neutralizes signaling by interleukin-5 (IL-5) and granulocyte-macrophage colony stimulating factor (GM-CSF);
(b) binds to an epitope formed upon dimerization of two CD131 polypeptides; and
(c) binds to residues within domain 1 of a CD131 polypeptide and residues within domain 4 of another CD131 polypeptide.

In one example, the CD131-binding protein comprises an antigen binding site, wherein the antigen binding site binds to or specifically binds to an epitope within Site 2 of CD131 and neutralizes signaling by IL-5 and GM-CSF. In this regard, the skilled artisan will be aware that Site 2 of CD131 is made up of residues from two CD131 polypeptides that form a dimer, e.g., Site 2 comprises residues within loops A-B and E-F of domain 1 of one CD131 polypeptide and residues within loops B-C and F-G of another CD131 polypeptide.

In one example, the CD131-binding protein comprises an antigen binding site, wherein the antigen binding site binds to an epitope formed upon dimerization of two CD131 polypeptides. For example, the antigen binding site binds to residues within domain 1 of a CD131 polypeptide and residues within domain 4 of another CD131 polypeptide.

In one example, the antigen binding site binds to an epitope comprising one or more of amino acids corresponding to residues 39 and/or 103 of SEQ ID NO: 1.

In one example, the antigen binding site binds to an epitope comprising one or more of amino acids corresponding to residues 338, 365, 367 and 368 of SEQ ID NO: 1.

In one example, the antigen binding site binds to an epitope formed upon dimerization of two CD131 polypeptides, wherein the epitope comprises one or more (or all) of amino acids corresponding to residues 39 and 103 of one CD131 polypeptide and residues 338, 365, 367 and 368 of the other CD131 polypeptide.

In one example, the compound is a CD131-binding protein that binds to one or more (or all) of the following mutant polypeptide(s):
(i) a mutant polypeptide comprising a sequence set forth in SEQ ID NO: 119;
(ii) a mutant polypeptide comprising a sequence set forth in SEQ ID NO: 123;
(iii) a mutant polypeptide comprising a sequence set forth in SEQ ID NO: 124;
(iv) a mutant polypeptide comprising a sequence set forth in SEQ ID NO: 135;
(v) a mutant polypeptide comprising a sequence set forth in SEQ ID NO: 131;
(vi) a mutant polypeptide comprising a sequence set forth in SEQ ID NO: 136;
(vii) a mutant polypeptide comprising a sequence set forth in SEQ ID NO: 137;
(viii) a mutant polypeptide comprising a sequence set forth in SEQ ID NO: 139;
(ix) a mutant polypeptide comprising a sequence set forth in SEQ ID NO: 145, at a level that is reduced compared to the level of binding of the compound that binds CD131 to a polypeptide comprising a sequence set forth in SEQ ID NO: 192.

In one example, the level of binding (e.g., as determined by K_{D}) of the CD131-binding protein to the mutant polypeptide is reduced by at least about 3 fold or 4 fold or fold or 10 fold. For example, the level of binding to the mutant polypeptide is reduced by at least about 20 fold or 50 fold or 100 fold.

In one example, the affinity (K_{D}) of the CD131-binding protein for the mutant polypeptide is about 4×10⁻⁶ or greater, e.g., 4.5×10⁻⁶ or 1×10⁻⁵.

In one example, the compound that binds to CD131 binds to or cross-reacts with a polypeptide comprising a sequence set forth in any one of SEQ ID NOs: 117, 118, 120-123, 125-130, 132-136, 138 or 140-148.

In one example, the compound that binds to CD131 binds to a polypeptide comprising a sequence set forth in SEQ ID NO: 127 with a higher affinity than it does to a polypeptide comprising a sequence set forth in SEQ ID NO: 192.

In one example, the compound is a CD131-binding protein that binds to or cross reacts with one or more (or all) of the following mutant polypeptide(s):
(i) a mutant polypeptide comprising a sequence set forth in SEQ ID NO: 135;
(ii) a mutant polypeptide comprising a sequence set forth in SEQ ID NO: 136; and/or
(iii) a mutant polypeptide comprising a sequence set forth in SEQ ID NO: 138.

Methods for determining binding of a CD131-binding protein to a polypeptide will be apparent to the skilled artisan. For example, the polypeptide is immobilized on a solid or semi-solid surface and the CD131-binding protein is contacted to the immobilized polypeptide. Binding is then determined, e.g., by surface plasmon resonance.

In one example, the CD131-binding protein comprising an antigen binding site is a protein comprising one or more antibody variable regions. In one example, the protein comprises a heavy chain variable region (V_{H}). In one example, the protein comprises a light chain variable region (V_{L}). In one example, the protein comprises a V_{H} and a V_{L}. In one example, the protein comprises a V_{H} and a V_{L}. In one example, the V_{H} and a V_{L} are in the same polypeptide chain. In another example, the V_{H} and a V_{L} are in separate polypeptide chains.

In one example, the protein is a single domain antibody (sdAb).

In one example, a CD131-binding protein described herein comprises at least a heavy chain variable region (V_{H}) and a light chain variable region (V_{L}), wherein the V_{H} and V_{L} bind to form a Fv comprising an antigen binding domain. The skilled artisan will understand that the antigen binding domain comprises the binding site of the antibody.

In one example, the V_{H} and the V_{L} are in a single polypeptide chain. For example, the protein is:
(i) a single chain Fv fragment (scFv);
(ii) a dimeric scFv (di-scFv);
(iii) one of (i) or (ii) linked to a constant region of an antibody, Fc or a heavy chain constant domain (C_{H}) 2 and/or C_{H}3; or
(iv) one of (i) or (ii) linked to a protein that binds to an immune effector cell.

In one example, the V_{L} and V_{H} are in separate polypeptide chains.

For example, the protein is:
(i) a diabody;
(ii) a triabody;
(iii) a tetrabody;
(iv) a Fab;
(v) a F(ab′)₂;
(vi) a Fv;
(vii) one of (i) to (vi) linked to a constant region of an antibody, Fc or a heavy chain constant domain (C_{H}) 2 and/or C_{H}3; or
(viii) one of (i) to (vi) linked to a protein that binds to an immune effector cell.

The foregoing proteins (described in the previous two lists) can also be referred to as antigen binding domains of antibodies.

In one example, the protein is an antibody, for example, a monoclonal antibody. In one example, the antibody is a naked antibody.

In one example, a protein (or antibody) is chimeric, de-immunized, humanized, human or primatized.

In one example, the protein or antibody is human. For example, the present disclosure provides an antibody which binds to or specifically binds to CD131 and neutralizes signaling by IL-5 and GM-CSF, and wherein the antibody comprises an antigen binding domain or a V_{H} and/or V_{L} as described herein in any example.

Exemplary antibodies include 9A2-VR24.29 (also, referred to as “CSL311”) described in WO 2017/088028.

In one example, the compound is a CD131-binding protein comprising an antigen binding domain of an antibody, wherein the antigen binding domain binds to or specifically binds to CD131 and neutralizes signalling by IL-5 and GM-CSF, and wherein the CD131-binding protein competitively inhibits binding of antibody 9A2 (comprising a V_{L} comprising a sequence set forth in SEQ ID NO: 5 and a V_{H} comprising a sequence set forth in SEQ ID NO: 20) to CD131.

In one example, the compound is a CD131-binding protein comprising an antigen binding domain of an antibody, wherein the antigen binding domain binds to or specifically binds to CD131 and neutralizes signalling by IL-5 and GM-CSF, and wherein the CD131-binding protein competitively inhibits binding of antibody 9A2 (comprising a V_{L} comprising a sequence set forth in SEQ ID NO: 5 and a human kappa light chain constant region and a V_{H} comprising a sequence set forth in SEQ ID NO: 20 and a human IgG4 constant region) to CD131.

In one example, the compound is a CD131-binding protein comprising an antigen binding domain of an antibody, wherein the antigen binding domain binds to or specifically binds to CD131 and neutralizes signalling by IL-5 and GM-CSF, and wherein the CD131-binding protein competitively inhibits binding of antibody 9A2 (comprising a light chain comprising a sequence set forth in SEQ ID NO: 5 and a heavy chain comprising a sequence set forth in SEQ ID NO: 20) to CD131.

In one example, the compound is a CD131-binding protein comprising an antigen binding domain of an antibody, wherein the antigen binding domain binds to a polypeptide comprising a sequence set forth in SEQ ID NO: 127 with a higher affinity than it does to a polypeptide comprising a sequence set forth in SEQ ID NO: 192.

In one example, the compound is a CD131-binding protein comprising an antigen binding domain of an antibody, wherein the antigen binding domain comprises a V_{H} and a V_{L}, wherein:
(A) (i) the V_{H} comprises a CDR1 comprising a sequence set forth between amino acids 26-35 of SEQ ID NO: 180, a CDR2 comprising a sequence set forth between amino acids 50-66 of SEQ ID NO: 180 and a CDR3 comprising a sequence set forth between amino acids 99-106 of SEQ ID NO: 180 or the V_{H} comprising a sequence set forth in SEQ ID NO: 180; and
   (ii) the V_{L} comprising a CDR1 comprising a sequence set forth between amino acids 24-34 of SEQ ID NO: 177, a CDR2 comprising a sequence set forth between amino acids 44-51 of SEQ ID NO: 177 and a CDR3 comprising a sequence set forth between amino acids 89-97 of SEQ ID NO: 177 or the V_{L} comprising a sequence set forth in SEQ ID NO: 177; or
(B) the V_{H} comprises a sequence set forth in SEQ ID NO: 193 and the V_{L} comprises a sequence set forth in SEQ ID NO: 5.

In one example, the compound is a CD131-binding protein comprising an antigen binding domain of an antibody, wherein the antigen binding domain comprises:
(i) a V_{H} comprising CDRs 1, 2 and 3 of a sequence set forth in SEQ ID NO: 20 and a V_{L} comprising CDRs 1, 2 and 3 of a sequence set forth in SEQ ID NO: 5;
(ii) a V_{H} comprising a sequence set forth in SEQ ID NO: 20 and a V_{L} comprising a sequence set forth in SEQ ID NO: 5;
(iii) a V_{H} comprising CDRs 1, 2 and 3 of a sequence set forth in SEQ ID NO: 20 and a V_{L} comprising CDRs 1, 2 and 3 of a sequence set forth in SEQ ID NO: 6;
(iv) a V_{H} comprising a sequence set forth in SEQ ID NO: 20 and a V_{L} comprising a sequence set forth in SEQ ID NO: 6;
(v) a V_{H} comprising CDRs 1, 2 and 3 of a sequence set forth in SEQ ID NO: 20 and a V_{L} comprising CDRs 1, 2 and 3 of a sequence set forth in SEQ ID NO: 7;
(vi) a V_{H} comprising a sequence set forth in SEQ ID NO: 20 and a V_{L} comprising a sequence set forth in SEQ ID NO: 7;
(vii) a V_{H} comprising CDRs 1, 2 and 3 of a sequence set forth in SEQ ID NO: 20 and a V_{L} comprising CDRs 1, 2 and 3 of a sequence set forth in SEQ ID NO: 8;
(viii) a V_{H} comprising a sequence set forth in SEQ ID NO: 20 and a V_{L} comprising a sequence set forth in SEQ ID NO: 8;
(ix) a V_{H} comprising CDRs 1, 2 and 3 of a sequence set forth in SEQ ID NO: 20 and a V_{L} comprising CDRs 1, 2 and 3 of a sequence set forth in SEQ ID NO: 9;
(x) a V_{H} comprising a sequence set forth in SEQ ID NO: 20 and a V_{L} comprising a sequence set forth in SEQ ID NO: 9;
(xi) a V_{H} comprising CDRs 1, 2 and 3 of a sequence set forth in SEQ ID NO: 20 and a V_{L} comprising CDRs 1, 2 and 3 of a sequence set forth in SEQ ID NO: 10;
(xii) a V_{H} comprising a sequence set forth in SEQ ID NO: 20 and a V_{L} comprising a sequence set forth in SEQ ID NO: 10;
(xiii) a V_{H} comprising CDRs 1, 2 and 3 of a sequence set forth in SEQ ID NO: 20 and a V_{L} comprising CDRs 1, 2 and 3 of a sequence set forth in SEQ ID NO: 11;
(xiv) a V_{H} comprising a sequence set forth in SEQ ID NO: 20 and a V_{L} comprising a sequence set forth in SEQ ID NO: 11;
(xv) a V_{H} comprising CDRs 1, 2 and 3 of a sequence set forth in SEQ ID NO: 20 and a V_{L} comprising CDRs 1, 2 and 3 of a sequence set forth in SEQ ID NO: 12;
(xvi) a V_{H} comprising a sequence set forth in SEQ ID NO: 20 and a V_{L} comprising a sequence set forth in SEQ ID NO: 12;
(xvii) a V_{H} comprising CDRs 1, 2 and 3 of a sequence set forth in SEQ ID NO: 20 and a V_{L} comprising CDRs 1, 2 and 3 of a sequence set forth in SEQ ID NO: 13;
(xviii) a V_{H} comprising a sequence set forth in SEQ ID NO: 20 and a V_{L} comprising a sequence set forth in SEQ ID NO: 13;
(xix) a V_{H} comprising CDRs 1, 2 and 3 of a sequence set forth in SEQ ID NO: 20 and a V_{L} comprising CDRs 1, 2 and 3 of a sequence set forth in SEQ ID NO: 14;
(xx) a V_{H} comprising a sequence set forth in SEQ ID NO: 20 and a V_{L} comprising a sequence set forth in SEQ ID NO: 14;
(xxi) a V_{H} comprising CDRs 1, 2 and 3 of a sequence set forth in SEQ ID NO: 20 and a V_{L} comprising CDRs 1, 2 and 3 of a sequence set forth in SEQ ID NO: 15;
(xxii) a V_{H} comprising a sequence set forth in SEQ ID NO: 20 and a V_{L} comprising a sequence set forth in SEQ ID NO: 15;
(xxiii) a V_{H} comprising CDRs 1, 2 and 3 of a sequence set forth in SEQ ID NO: 21 and a V_{L} comprising CDRs 1, 2 and 3 of a sequence set forth in SEQ ID NO: 5;
(xxiv) a V_{H} comprising a sequence set forth in SEQ ID NO: 21 and a V_{L} comprising a sequence set forth in SEQ ID NO: 5;
(xxv) a V_{H} comprising CDRs 1, 2 and 3 of a sequence set forth in SEQ ID NO: 22 and a V_{L} comprising CDRs 1, 2 and 3 of a sequence set forth in SEQ ID NO: 5;
(xxvi) a V_{H} comprising a sequence set forth in SEQ ID NO: 22 and a V_{L} comprising a sequence set forth in SEQ ID NO: 5;
(xxvii) a V_{H} comprising CDRs 1, 2 and 3 of a sequence set forth in SEQ ID NO: 23 and a V_{L} comprising CDRs 1, 2 and 3 of a sequence set forth in SEQ ID NO: 5;
(xxviii) a V_{H} comprising a sequence set forth in SEQ ID NO: 23 and a V_{L} comprising a sequence set forth in SEQ ID NO: 5;
(xxix) a V_{H} comprising CDRs 1, 2 and 3 of a sequence set forth in SEQ ID NO: 24 and a V_{L} comprising CDRs 1, 2 and 3 of a sequence set forth in SEQ ID NO: 5;
(xxx) a V_{H} comprising a sequence set forth in SEQ ID NO: 24 and a V_{L} comprising a sequence set forth in SEQ ID NO: 5;
(xxxi) a V_{H} comprising CDRs 1, 2 and 3 of a sequence set forth in SEQ ID NO: 25 and a V_{L} comprising CDRs 1, 2 and 3 of a sequence set forth in SEQ ID NO: 5;
(xxxii) a V_{H} comprising a sequence set forth in SEQ ID NO: 25 and a V_{L} comprising a sequence set forth in SEQ ID NO: 5;
(xxxiii) a V_{H} comprising CDRs 1, 2 and 3 of a sequence set forth in SEQ ID NO: 26 and a V_{L} comprising CDRs 1, 2 and 3 of a sequence set forth in SEQ ID NO: 5;
(xxxiv) a V_{H} comprising a sequence set forth in SEQ ID NO: 26 and a V_{L} comprising a sequence set forth in SEQ ID NO: 5;
(xxxv) a V_{H} comprising CDRs 1, 2 and 3 of a sequence set forth in SEQ ID NO: 27 and a V_{L} comprising CDRs 1, 2 and 3 of a sequence set forth in SEQ ID NO: 5;
(xxxvi) a V_{H} comprising a sequence set forth in SEQ ID NO: 27 and a V_{L} comprising a sequence set forth in SEQ ID NO: 5;
(xxxvii) a V_{H} comprising CDRs 1, 2 and 3 of a sequence set forth in SEQ ID NO: 28 and a V_{L} comprising CDRs 1, 2 and 3 of a sequence set forth in SEQ ID NO: 5;
(xxxviii) a V_{H} comprising a sequence set forth in SEQ ID NO: 28 and a V_{L} comprising a sequence set forth in SEQ ID NO: 5;
(xxxix) a V_{H} comprising CDRs 1, 2 and 3 of a sequence set forth in SEQ ID NO: 29 and a V_{L} comprising CDRs 1, 2 and 3 of a sequence set forth in SEQ ID NO: 5;
(xl) a V_{H} comprising a sequence set forth in SEQ ID NO: 29 and a V_{L} comprising a sequence set forth in SEQ ID NO: 5;
(xli) a V_{H} comprising CDRs 1, 2 and 3 of a sequence set forth in SEQ ID NO: 30 and a V_{L} comprising CDRs 1, 2 and 3 of a sequence set forth in SEQ ID NO: 5;
(xlii) a V_{H} comprising a sequence set forth in SEQ ID NO: 30 and a V_{L} comprising a sequence set forth in SEQ ID NO: 5;
(xliii) a V_{H} comprising CDRs 1, 2 and 3 of a sequence set forth in SEQ ID NO: 31 and a V_{L} comprising CDRs 1, 2 and 3 of a sequence set forth in SEQ ID NO: 5;
(xliv) a V_{H} comprising a sequence set forth in SEQ ID NO: 31 and a V_{L} comprising a sequence set forth in SEQ ID NO: 5;
(xlv) a V_{H} comprising CDRs 1, 2 and 3 of a sequence set forth in SEQ ID NO: 32 and a V_{L} comprising CDRs 1, 2 and 3 of a sequence set forth in SEQ ID NO: 5;
(xlvi) a V_{H} comprising a sequence set forth in SEQ ID NO: 32 and a V_{L} comprising a sequence set forth in SEQ ID NO: 5;
(xlvii) a V_{H} comprising CDRs 1, 2 and 3 of a sequence set forth in SEQ ID NO: 33 and a V_{L} comprising CDRs 1, 2 and 3 of a sequence set forth in SEQ ID NO: 5;
(xlviii) a V_{H} comprising a sequence set forth in SEQ ID NO: 33 and a V_{L} comprising a sequence set forth in SEQ ID NO: 5;
(xlix) a V_{H} comprising CDRs 1, 2 and 3 of a sequence set forth in SEQ ID NO: 34 and a V_{L} comprising CDRs 1, 2 and 3 of a sequence set forth in SEQ ID NO: 5;
(l) a V_{H} comprising a sequence set forth in SEQ ID NO: 34 and a V_{L} comprising a sequence set forth in SEQ ID NO: 5;
(li) a V_{H} comprising CDRs 1, 2 and 3 of a sequence set forth in SEQ ID NO: 35 and a V_{L} comprising CDRs 1, 2 and 3 of a sequence set forth in SEQ ID NO: 5;
(lii) a V_{H} comprising a sequence set forth in SEQ ID NO: 35 and a V_{L} comprising a sequence set forth in SEQ ID NO: 5;
(liii) a V_{H} comprising CDRs 1, 2 and 3 of a sequence set forth in SEQ ID NO: 36 and a V_{L} comprising CDRs 1, 2 and 3 of a sequence set forth in SEQ ID NO: 5;
(liv) a V_{H} comprising a sequence set forth in SEQ ID NO: 36 and a V_{L} comprising a sequence set forth in SEQ ID NO: 5;
(lv) a V_{H} comprising CDRs 1, 2 and 3 of a sequence set forth in SEQ ID NO: 37 and a V_{L} comprising CDRs 1, 2 and 3 of a sequence set forth in SEQ ID NO: 5;
(lvi) a V_{H} comprising a sequence set forth in SEQ ID NO: 37 and a V_{L} comprising a sequence set forth in SEQ ID NO: 5;
(lvii) a V_{H} comprising CDRs 1, 2 and 3 of a sequence set forth in SEQ ID NO: 38 and a V_{L} comprising CDRs 1, 2 and 3 of a sequence set forth in SEQ ID NO: 5;
(lviii) a V_{H} comprising a sequence set forth in SEQ ID NO: 38 and a V_{L} comprising a sequence set forth in SEQ ID NO: 5;
(lix) a V_{H} comprising CDRs 1, 2 and 3 of a sequence set forth in SEQ ID NO: 39 and a V_{L} comprising CDRs 1, 2 and 3 of a sequence set forth in SEQ ID NO: 5;
(lx) a V_{H} comprising a sequence set forth in SEQ ID NO: 39 and a V_{L} comprising a sequence set forth in SEQ ID NO: 5;
(lxi) a V_{H} comprising CDRs 1, 2 and 3 of a sequence set forth in SEQ ID NO: 40 and a V_{L} comprising CDRs 1, 2 and 3 of a sequence set forth in SEQ ID NO: 5;
(lxii) a V_{H} comprising a sequence set forth in SEQ ID NO: 40 and a V_{L} comprising a sequence set forth in SEQ ID NO: 5;
(lxiii) a V_{H} comprising CDRs 1, 2 and 3 of a sequence set forth in SEQ ID NO: 41 and a V_{L} comprising CDRs 1, 2 and 3 of a sequence set forth in SEQ ID NO: 5;
(lxiv) a V_{H} comprising a sequence set forth in SEQ ID NO: 41 and a V_{L} comprising a sequence set forth in SEQ ID NO: 5;
(lxv) a V_{H} comprising CDRs 1, 2 and 3 of a sequence set forth in SEQ ID NO: 42 and a V_{L} comprising CDRs 1, 2 and 3 of a sequence set forth in SEQ ID NO: 5;
(lxvi) a V_{H} comprising a sequence set forth in SEQ ID NO: 42 and a V_{L} comprising a sequence set forth in SEQ ID NO: 5;
(lxvii) a V_{H} comprising CDRs 1, 2 and 3 of a sequence set forth in SEQ ID NO: 43 and a V_{L} comprising CDRs 1, 2 and 3 of a sequence set forth in SEQ ID NO: 5;
(lxviii) a V_{H} comprising a sequence set forth in SEQ ID NO: 43 and a V_{L} comprising a sequence set forth in SEQ ID NO: 5;
(lxix) a V_{H} comprising CDRs 1, 2 and 3 of a sequence set forth in SEQ ID NO: 44 and a V_{L} comprising CDRs 1, 2 and 3 of a sequence set forth in SEQ ID NO: 5;
(lxx) a V_{H} comprising a sequence set forth in SEQ ID NO: 44 and a V_{L} comprising a sequence set forth in SEQ ID NO: 5;
(lxxi) a V_{H} comprising CDRs 1, 2 and 3 of a sequence set forth in SEQ ID NO: 45 and a V_{L} comprising CDRs 1, 2 and 3 of a sequence set forth in SEQ ID NO: 5;
(lxxii) a V_{H} comprising a sequence set forth in SEQ ID NO: 45 and a V_{L} comprising a sequence set forth in SEQ ID NO: 5;
(lxxiii) a V_{H} comprising CDRs 1, 2 and 3 of a sequence set forth in SEQ ID NO: 46 and a V_{L} comprising CDRs 1, 2 and 3 of a sequence set forth in SEQ ID NO: 5;
(lxxiv) a V_{H} comprising a sequence set forth in SEQ ID NO: 46 and a V_{L} comprising a sequence set forth in SEQ ID NO: 5;
(lxxv) a V_{H} comprising CDRs 1, 2 and 3 of a sequence set forth in SEQ ID NO: 47 and a V_{L} comprising CDRs 1, 2 and 3 of a sequence set forth in SEQ ID NO: 5;
(lxxvi) a V_{H} comprising a sequence set forth in SEQ ID NO: 47 and a V_{L} comprising a sequence set forth in SEQ ID NO: 5;
(lxxvii) a V_{H} comprising CDRs 1, 2 and 3 of a sequence set forth in SEQ ID NO: 48 and a V_{L} comprising CDRs 1, 2 and 3 of a sequence set forth in SEQ ID NO: 5;
(lxxviii) a V_{H} comprising a sequence set forth in SEQ ID NO: 48 and a V_{L} comprising a sequence set forth in SEQ ID NO: 5;
(lxxix) a V_{H} comprising CDRs 1, 2 and 3 of a sequence set forth in SEQ ID NO: 49 and a V_{L} comprising CDRs 1, 2 and 3 of a sequence set forth in SEQ ID NO: 5;
(lxxx) a V_{H} comprising a sequence set forth in SEQ ID NO: 49 and a V_{L} comprising a sequence set forth in SEQ ID NO: 5;
(lxxxi) a V_{H} comprising CDRs 1, 2 and 3 of a sequence set forth in SEQ ID NO: 50 and a V_{L} comprising CDRs 1, 2 and 3 of a sequence set forth in SEQ ID NO: 5;
(lxxxii) a V_{H} comprising a sequence set forth in SEQ ID NO: 50 and a V_{L} comprising a sequence set forth in SEQ ID NO: 5;
(lxxxiii) a V_{H} comprising CDRs 1, 2 and 3 of a sequence set forth in SEQ ID NO: 51 and a V_{L} comprising CDRs 1, 2 and 3 of a sequence set forth in SEQ ID NO: 5;
(lxxxiv) a V_{H} comprising a sequence set forth in SEQ ID NO: 51 and a V_{L} comprising a sequence set forth in SEQ ID NO: 5;
(lxxxv) a V_{H} comprising CDRs 1, 2 and 3 of a sequence set forth in SEQ ID NO: 52 and a V_{L} comprising CDRs 1, 2 and 3 of a sequence set forth in SEQ ID NO: 5;
(lxxxvi) a V_{H} comprising a sequence set forth in SEQ ID NO: 52 and a V_{L} comprising a sequence set forth in SEQ ID NO: 5;
(lxxxvii) a V_{H} comprising CDRs 1, 2 and 3 of a sequence set forth in SEQ ID NO: 53 and a V_{L} comprising CDRs 1, 2 and 3 of a sequence set forth in SEQ ID NO: 5;
(lxxxviii) a V_{H} comprising a sequence set forth in SEQ ID NO: 53 and a V_{L} comprising a sequence set forth in SEQ ID NO: 5;
(lxxxix) a V_{H} comprising CDRs 1, 2 and 3 of a sequence set forth in SEQ ID NO: 54 and a V_{L} comprising CDRs 1, 2 and 3 of a sequence set forth in SEQ ID NO: 5;
(xc) a V_{H} comprising a sequence set forth in SEQ ID NO: 54 and a V_{L} comprising a sequence set forth in SEQ ID NO: 5;
(xci) a V_{H} comprising CDRs 1, 2 and 3 of a sequence set forth in SEQ ID NO: 55 and a V_{L} comprising CDRs 1, 2 and 3 of a sequence set forth in SEQ ID NO: 5;
(xcii) a V_{H} comprising a sequence set forth in SEQ ID NO: 55 and a V_{L} comprising a sequence set forth in SEQ ID NO: 5;
(xciii) a V_{H} comprising CDRs 1, 2 and 3 of a sequence set forth in SEQ ID NO: 56 and a V_{L} comprising CDRs 1, 2 and 3 of a sequence set forth in SEQ ID NO: 5;
(xciv) a V_{H} comprising a sequence set forth in SEQ ID NO: 56 and a V_{L} comprising a sequence set forth in SEQ ID NO: 5;
(xcv) a V_{H} comprising CDRs 1, 2 and 3 of a sequence set forth in SEQ ID NO: 57 and a V_{L} comprising CDRs 1, 2 and 3 of a sequence set forth in SEQ ID NO: 5;
(xcvi) a V_{H} comprising a sequence set forth in SEQ ID NO: 57 and a V_{L} comprising a sequence set forth in SEQ ID NO: 5;
(xcvii) a V_{H} comprising CDRs 1, 2 and 3 of a sequence set forth in SEQ ID NO: 58 and a V_{L} comprising CDRs 1, 2 and 3 of a sequence set forth in SEQ ID NO: 5;
(xcviii) a V_{H} comprising a sequence set forth in SEQ ID NO: 58 and a V_{L} comprising a sequence set forth in SEQ ID NO: 5;
(xcix) a V_{H} comprising CDRs 1, 2 and 3 of a sequence set forth in SEQ ID NO: 59 and a V_{L} comprising CDRs 1, 2 and 3 of a sequence set forth in SEQ ID NO: 5;
(c) a V_{H} comprising a sequence set forth in SEQ ID NO: 59 and a V_{L} comprising a sequence set forth in SEQ ID NO: 5;
(ci) a V_{H} comprising CDRs 1, 2 and 3 of a sequence set forth in SEQ ID NO: 60 and a V_{L} comprising CDRs 1, 2 and 3 of a sequence set forth in SEQ ID NO: 5;
(cii) a V_{H} comprising a sequence set forth in SEQ ID NO: 60 and a V_{L} comprising a sequence set forth in SEQ ID NO: 5;
(ciii) a V_{H} comprising CDRs 1, 2 and 3 of a sequence set forth in SEQ ID NO: 61 and a V_{L} comprising CDRs 1, 2 and 3 of a sequence set forth in SEQ ID NO: 5;
(civ) a V_{H} comprising a sequence set forth in SEQ ID NO: 61 and a V_{L} comprising a sequence set forth in SEQ ID NO: 5;
(cv) a V_{H} comprising CDRs 1, 2 and 3 of a sequence set forth in SEQ ID NO: 62 and a V_{L} comprising CDRs 1, 2 and 3 of a sequence set forth in SEQ ID NO: 5;
(cvi) a V_{H} comprising a sequence set forth in SEQ ID NO: 62 and a V_{L} comprising a sequence set forth in SEQ ID NO: 5;
(cvii) a V_{H} comprising CDRs 1, 2 and 3 of a sequence set forth in SEQ ID NO: 63 and a V_{L} comprising CDRs 1, 2 and 3 of a sequence set forth in SEQ ID NO: 5;
(cviii) a V_{H} comprising a sequence set forth in SEQ ID NO: 63 and a V_{L} comprising a sequence set forth in SEQ ID NO: 5;
(cix) a V_{H} comprising CDRs 1, 2 and 3 of a sequence set forth in SEQ ID NO: 64 and a V_{L} comprising CDRs 1, 2 and 3 of a sequence set forth in SEQ ID NO: 5;
(cx) a V_{H} comprising a sequence set forth in SEQ ID NO: 64 and a V_{L} comprising a sequence set forth in SEQ ID NO: 5;
(cxi) a V_{H} comprising CDRs 1, 2 and 3 of a sequence set forth in SEQ ID NO: 65 and a V_{L} comprising CDRs 1, 2 and 3 of a sequence set forth in SEQ ID NO: 5;
(cxii) a V_{H} comprising a sequence set forth in SEQ ID NO: 65 and a V_{L} comprising a sequence set forth in SEQ ID NO: 5;
(cxiii) a V_{H} comprising CDRs 1, 2 and 3 of a sequence set forth in SEQ ID NO: 66 and a V_{L} comprising CDRs 1, 2 and 3 of a sequence set forth in SEQ ID NO: 5;
(cxiv) a V_{H} comprising a sequence set forth in SEQ ID NO: 66 and a V_{L} comprising a sequence set forth in SEQ ID NO: 5;
(cxv) a V_{H} comprising CDRs 1, 2 and 3 of a sequence set forth in SEQ ID NO: 67 and a V_{L} comprising CDRs 1, 2 and 3 of a sequence set forth in SEQ ID NO: 5;
(cxvi) a V_{H} comprising a sequence set forth in SEQ ID NO: 67 and a V_{L} comprising a sequence set forth in SEQ ID NO: 5;
(cxvii) a V_{H} comprising CDRs 1, 2 and 3 of a sequence set forth in SEQ ID NO: 68 and a V_{L} comprising CDRs 1, 2 and 3 of a sequence set forth in SEQ ID NO: 5;
(cxviii) a V_{H} comprising a sequence set forth in SEQ ID NO: 68 and a V_{L} comprising a sequence set forth in SEQ ID NO: 5;
(cxix) a V_{H} comprising CDRs 1, 2 and 3 of a sequence set forth in SEQ ID NO: 69 and a V_{L} comprising CDRs 1, 2 and 3 of a sequence set forth in SEQ ID NO: 5;
(cxx) a V_{H} comprising a sequence set forth in SEQ ID NO: 69 and a V_{L} comprising a sequence set forth in SEQ ID NO: 5;
(cxxi) a V_{H} comprising CDRs 1, 2 and 3 of a sequence set forth in SEQ ID NO: 70 and a V_{L} comprising CDRs 1, 2 and 3 of a sequence set forth in SEQ ID NO: 5;
(cxxii) a V_{H} comprising a sequence set forth in SEQ ID NO: 70 and a V_{L} comprising a sequence set forth in SEQ ID NO: 5;
(cxxiii) a V_{H} comprising CDRs 1, 2 and 3 of a sequence set forth in SEQ ID NO: 71 and a V_{L} comprising CDRs 1, 2 and 3 of a sequence set forth in SEQ ID NO: 5;
(cxxiv) a V_{H} comprising a sequence set forth in SEQ ID NO: 71 and a V_{L} comprising a sequence set forth in SEQ ID NO: 5;
(cxxv) a V_{H} comprising CDRs 1, 2 and 3 of a sequence set forth in SEQ ID NO: 72 and a V_{L} comprising CDRs 1, 2 and 3 of a sequence set forth in SEQ ID NO: 5;
(cxxvi) a V_{H} comprising a sequence set forth in SEQ ID NO: 72 and a V_{L} comprising a sequence set forth in SEQ ID NO: 5;
(cxxvii) a V_{H} comprising CDRs 1, 2 and 3 of a sequence set forth in SEQ ID NO: 73 and a V_{L} comprising CDRs 1, 2 and 3 of a sequence set forth in SEQ ID NO: 5;
(cxxviii) a V_{H} comprising a sequence set forth in SEQ ID NO: 73 and a V_{L} comprising a sequence set forth in SEQ ID NO: 5;
(cxxix) a V_{H} comprising CDRs 1, 2 and 3 of a sequence set forth in SEQ ID NO: 75 and a V_{L} comprising CDRs 1, 2 and 3 of a sequence set forth in SEQ ID NO: 5;
(cxxx) a V_{H} comprising a sequence set forth in SEQ ID NO: 75 and a V_{L} comprising a sequence set forth in SEQ ID NO: 5;
(cxxxi) a V_{H} comprising CDRs 1, 2 and 3 of a sequence set forth in SEQ ID NO: 76 and a V_{L} comprising CDRs 1, 2 and 3 of a sequence set forth in SEQ ID NO: 5;
(cxxxii) a V_{H} comprising a sequence set forth in SEQ ID NO: 76 and a V_{L} comprising a sequence set forth in SEQ ID NO: 5;
(cxxxiii) a V_{H} comprising CDRs 1, 2 and 3 of a sequence set forth in SEQ ID NO: 77 and a V_{L} comprising CDRs 1, 2 and 3 of a sequence set forth in SEQ ID NO: 5;
(cxxxiv) a V_{H} comprising a sequence set forth in SEQ ID NO: 77 and a V_{L} comprising a sequence set forth in SEQ ID NO: 5;
(cxxxv) a V_{H} comprising CDRs 1, 2 and 3 of a sequence set forth in SEQ ID NO: 78 and a V_{L} comprising CDRs 1, 2 and 3 of a sequence set forth in SEQ ID NO: 5;
(cxxxvi) a V_{H} comprising a sequence set forth in SEQ ID NO: 78 and a V_{L} comprising a sequence set forth in SEQ ID NO: 5;
(cxxxvii) a V_{H} comprising CDRs 1, 2 and 3 of a sequence set forth in SEQ ID NO: 79 and a V_{L} comprising CDRs 1, 2 and 3 of a sequence set forth in SEQ ID NO: 5;
(cxxxviii) a V_{H} comprising a sequence set forth in SEQ ID NO: 79 and a V_{L} comprising a sequence set forth in SEQ ID NO: 5;
(cxxxix) a V_{H} comprising CDRs 1, 2 and 3 of a sequence set forth in SEQ ID NO: 80 and a V_{L} comprising CDRs 1, 2 and 3 of a sequence set forth in SEQ ID NO: 5;
(cxl) a V_{H} comprising a sequence set forth in SEQ ID NO: 80 and a V_{L} comprising a sequence set forth in SEQ ID NO: 5;
(cxli) a V_{H} comprising CDRs 1, 2 and 3 of a sequence set forth in SEQ ID NO: 81 and a V_{L} comprising CDRs 1, 2 and 3 of a sequence set forth in SEQ ID NO: 5;
(cxlii) a V_{H} comprising a sequence set forth in SEQ ID NO: 81 and a V_{L} comprising a sequence set forth in SEQ ID NO: 5;
(cxliii) a V_{H} comprising CDRs 1, 2 and 3 of a sequence set forth in SEQ ID NO: 82 and a V_{L} comprising CDRs 1, 2 and 3 of a sequence set forth in SEQ ID NO: 5;
(cxliv) a V_{H} comprising a sequence set forth in SEQ ID NO: 82 and a V_{L} comprising a sequence set forth in SEQ ID NO: 5;
(cxlv) a V_{H} comprising CDRs 1, 2 and 3 of a sequence set forth in SEQ ID NO: 83 and a V_{L} comprising CDRs 1, 2 and 3 of a sequence set forth in SEQ ID NO: 5;
(cxlvi) a V_{H} comprising a sequence set forth in SEQ ID NO: 83 and a V_{L} comprising a sequence set forth in SEQ ID NO: 5;
(cxlvii) a V_{H} comprising CDRs 1, 2 and 3 of a sequence set forth in SEQ ID NO: 84 and a V_{L} comprising CDRs 1, 2 and 3 of a sequence set forth in SEQ ID NO: 5;
(cxlviii) a V_{H} comprising a sequence set forth in SEQ ID NO: 84 and a V_{L} comprising a sequence set forth in SEQ ID NO: 5;
(cxlix) a V_{H} comprising CDRs 1, 2 and 3 of a sequence set forth in SEQ ID NO: 85 and a V_{L} comprising CDRs 1, 2 and 3 of a sequence set forth in SEQ ID NO: 5;
(cl) a V_{H} comprising a sequence set forth in SEQ ID NO: 85 and a V_{L} comprising a sequence set forth in SEQ ID NO: 5;
(cli) a V_{H} comprising CDRs 1, 2 and 3 of a sequence set forth in SEQ ID NO: 86 and a V_{L} comprising CDRs 1, 2 and 3 of a sequence set forth in SEQ ID NO: 5;
(clii) a V_{H} comprising a sequence set forth in SEQ ID NO: 86 and a V_{L} comprising a sequence set forth in SEQ ID NO: 5;
(cliii) a V_{H} comprising CDRs 1, 2 and 3 of a sequence set forth in SEQ ID NO: 87 and a V_{L} comprising CDRs 1, 2 and 3 of a sequence set forth in SEQ ID NO: 5;
(cliv) a V_{H} comprising a sequence set forth in SEQ ID NO: 87 and a V_{L} comprising a sequence set forth in SEQ ID NO: 5;
(clv) a V_{H} comprising CDRs 1, 2 and 3 of a sequence set forth in SEQ ID NO: 88 and a V_{L} comprising CDRs 1, 2 and 3 of a sequence set forth in SEQ ID NO: 5;
(clvi) a V_{H} comprising a sequence set forth in SEQ ID NO: 88 and a V_{L} comprising a sequence set forth in SEQ ID NO: 5;
(clvii) a V_{H} comprising CDRs 1, 2 and 3 of a sequence set forth in SEQ ID NO: 89 and a V_{L} comprising CDRs 1, 2 and 3 of a sequence set forth in SEQ ID NO: 5;
(clviii) a V_{H} comprising a sequence set forth in SEQ ID NO: 89 and a V_{L} comprising a sequence set forth in SEQ ID NO: 5;
(clix) a V_{H} comprising CDRs 1, 2 and 3 of a sequence set forth in SEQ ID NO: 90 and a V_{L} comprising CDRs 1, 2 and 3 of a sequence set forth in SEQ ID NO: 5;
(clx) a V_{H} comprising a sequence set forth in SEQ ID NO: 90 and a V_{L} comprising a sequence set forth in SEQ ID NO: 5;
(clxi) a V_{H} comprising CDRs 1, 2 and 3 of a sequence set forth in SEQ ID NO: 91 and a V_{L} comprising CDRs 1, 2 and 3 of a sequence set forth in SEQ ID NO: 5;
(clxii) a V_{H} comprising a sequence set forth in SEQ ID NO: 91 and a V_{L} comprising a sequence set forth in SEQ ID NO: 5;
(clxiii) a V_{H} comprising CDRs 1, 2 and 3 of a sequence set forth in SEQ ID NO: 92 and a V_{L} comprising CDRs 1, 2 and 3 of a sequence set forth in SEQ ID NO: 5;
(clxiv) a V_{H} comprising a sequence set forth in SEQ ID NO: 92 and a V_{L} comprising a sequence set forth in SEQ ID NO: 5;
(clxv) a V_{H} comprising CDRs 1, 2 and 3 of a sequence set forth in SEQ ID NO: 93 and a V_{L} comprising CDRs 1, 2 and 3 of a sequence set forth in SEQ ID NO: 5;
(clxvi) a V_{H} comprising a sequence set forth in SEQ ID NO: 93 and a V_{L} comprising a sequence set forth in SEQ ID NO: 5;
(clxvii) a V_{H} comprising CDRs 1, 2 and 3 of a sequence set forth in SEQ ID NO: 94 and a V_{L} comprising CDRs 1, 2 and 3 of a sequence set forth in SEQ ID NO: 5;
(clxviii) a V_{H} comprising a sequence set forth in SEQ ID NO: 94 and a V_{L} comprising a sequence set forth in SEQ ID NO: 5;
(clxix) a V_{H} comprising CDRs 1, 2 and 3 of a sequence set forth in SEQ ID NO: 95 and a V_{L} comprising CDRs 1, 2 and 3 of a sequence set forth in SEQ ID NO: 5;
(clxx) a V_{H} comprising a sequence set forth in SEQ ID NO: 95 and a V_{L} comprising a sequence set forth in SEQ ID NO: 5;
(clxxi) a V_{H} comprising CDRs 1, 2 and 3 of a sequence set forth in SEQ ID NO: 96 and a V_{L} comprising CDRs 1, 2 and 3 of a sequence set forth in SEQ ID NO: 5;
(clxxii) a V_{H} comprising a sequence set forth in SEQ ID NO: 96 and a V_{L} comprising a sequence set forth in SEQ ID NO: 5;
(clxxiii) a V_{H} comprising CDRs 1, 2 and 3 of a sequence set forth in SEQ ID NO: 97 and a V_{L} comprising CDRs 1, 2 and 3 of a sequence set forth in SEQ ID NO: 5;
(clxxiv) a V_{H} comprising a sequence set forth in SEQ ID NO: 97 and a V_{L} comprising a sequence set forth in SEQ ID NO: 5;
(clxxv) a V_{H} comprising CDRs 1, 2 and 3 of a sequence set forth in SEQ ID NO: 98 and a V_{L} comprising CDRs 1, 2 and 3 of a sequence set forth in SEQ ID NO: 5;
(clxxvi) a V_{H} comprising a sequence set forth in SEQ ID NO: 98 and a V_{L} comprising a sequence set forth in SEQ ID NO: 5;
(clxxvii) a V_{H} comprising CDRs 1, 2 and 3 of a sequence set forth in SEQ ID NO: 99 and a V_{L} comprising CDRs 1, 2 and 3 of a sequence set forth in SEQ ID NO: 5;
(clxxviii) a V_{H} comprising a sequence set forth in SEQ ID NO: 99 and a V_{L} comprising a sequence set forth in SEQ ID NO: 5; (clxxix) a V_{H} comprising CDRs 1, 2 and 3 of a sequence set forth in SEQ ID NO: 100 and a V_{L} comprising CDRs 1, 2 and 3 of a sequence set forth in SEQ ID NO: 5;
(clxxx) a V_{H} comprising a sequence set forth in SEQ ID NO: 100 and a V_{L} comprising a sequence set forth in SEQ ID NO: 5;
(clxxxi) a V_{H} comprising CDRs 1, 2 and 3 of a sequence set forth in SEQ ID NO: 101 and a V_{L} comprising CDRs 1, 2 and 3 of a sequence set forth in SEQ ID NO: 5;
(clxxxii) a V_{H} comprising a sequence set forth in SEQ ID NO: 101 and a V_{L} comprising a sequence set forth in SEQ ID NO: 5;
(clxxxiii) a V_{H} comprising CDRs 1, 2 and 3 of a sequence set forth in SEQ ID NO: 102 and a V_{L} comprising CDRs 1, 2 and 3 of a sequence set forth in SEQ ID NO: 5;
(clxxxiv) a V_{H} comprising a sequence set forth in SEQ ID NO: 102 and a V_{L} comprising a sequence set forth in SEQ ID NO: 5;
(clxxxv) a V_{H} comprising CDRs 1, 2 and 3 of a sequence set forth in SEQ ID NO: 103 and a V_{L} comprising CDRs 1, 2 and 3 of a sequence set forth in SEQ ID NO: 5;
(clxxxvi) a V_{H} comprising a sequence set forth in SEQ ID NO: 103 and a V_{L} comprising a sequence set forth in SEQ ID NO: 5;
(clxxxvii) a V_{H} comprising CDRs 1, 2 and 3 of a sequence set forth in SEQ ID NO: 104 and a V_{L} comprising CDRs 1, 2 and 3 of a sequence set forth in SEQ ID NO: 5;
(clxxxviii) a V_{H} comprising a sequence set forth in SEQ ID NO: 104 and a V_{L} comprising a sequence set forth in SEQ ID NO: 5;
(clxxxix) a V_{H} comprising CDRs 1, 2 and 3 of a sequence set forth in SEQ ID NO: 105 and a V_{L} comprising CDRs 1, 2 and 3 of a sequence set forth in SEQ ID NO: 5;
(cxc) a V_{H} comprising a sequence set forth in SEQ ID NO: 105 and a V_{L} comprising a sequence set forth in SEQ ID NO: 5;
(cxci) a V_{H} comprising CDRs 1, 2 and 3 of a sequence set forth in SEQ ID NO: 106 and a V_{L} comprising CDRs 1, 2 and 3 of a sequence set forth in SEQ ID NO: 5;
(cxcii) a V_{H} comprising a sequence set forth in SEQ ID NO: 106 and a V_{L} comprising a sequence set forth in SEQ ID NO: 5;
(cxciii) a V_{H} comprising CDRs 1, 2 and 3 of a sequence set forth in SEQ ID NO: 107 and a V_{L} comprising CDRs 1, 2 and 3 of a sequence set forth in SEQ ID NO: 5;
(cxciv) a V_{H} comprising a sequence set forth in SEQ ID NO: 107 and a V_{L} comprising a sequence set forth in SEQ ID NO: 5;
(cxcv) a V_{H} comprising CDRs 1, 2 and 3 of a sequence set forth in SEQ ID NO: 108 and a V_{L} comprising CDRs 1, 2 and 3 of a sequence set forth in SEQ ID NO: 5;
(cxcvi) a V_{H} comprising a sequence set forth in SEQ ID NO: 108 and a V_{L} comprising a sequence set forth in SEQ ID NO: 5;
(cxcvii) a V_{H} comprising CDRs 1, 2 and 3 of a sequence set forth in SEQ ID NO: 109 and a V_{L} comprising CDRs 1, 2 and 3 of a sequence set forth in SEQ ID NO: 5;
(cxcviii) a V_{H} comprising a sequence set forth in SEQ ID NO: 109 and a V_{L} comprising a sequence set forth in SEQ ID NO: 5;
(cxcix) a V_{H} comprising CDRs 1, 2 and 3 of a sequence set forth in SEQ ID NO: 110 and a V_{L} comprising CDRs 1, 2 and 3 of a sequence set forth in SEQ ID NO: 5;
(cc) a V_{H} comprising a sequence set forth in SEQ ID NO: 110 and a V_{L} comprising a sequence set forth in SEQ ID NO: 5;
(cci) a V_{H} comprising CDRs 1, 2 and 3 of a sequence set forth in SEQ ID NO: 111 and a V_{L} comprising CDRs 1, 2 and 3 of a sequence set forth in SEQ ID NO: 5;
(ccii) a V_{H} comprising a sequence set forth in SEQ ID NO: 111 and a V_{L} comprising a sequence set forth in SEQ ID NO: 5;
(cciii) a V_{H} comprising CDRs 1, 2 and 3 of a sequence set forth in SEQ ID NO: 112 and a V_{L} comprising CDRs 1, 2 and 3 of a sequence set forth in SEQ ID NO: 5;
(cciv) a V_{H} comprising a sequence set forth in SEQ ID NO: 112 and a V_{L} comprising a sequence set forth in SEQ ID NO: 5;
(ccv) a V_{H} comprising CDRs 1, 2 and 3 of a sequence set forth in SEQ ID NO: 113 and a V_{L} comprising CDRs 1, 2 and 3 of a sequence set forth in SEQ ID NO: 5;
(ccvi) a V_{H} comprising a sequence set forth in SEQ ID NO: 113 and a V_{L} comprising a sequence set forth in SEQ ID NO: 5;
(ccvii) a V_{H} comprising CDRs 1, 2 and 3 of a sequence set forth in SEQ ID NO: 114 and a V_{L} comprising CDRs 1, 2 and 3 of a sequence set forth in SEQ ID NO: 5;
(ccviii) a V_{H} comprising a sequence set forth in SEQ ID NO: 114 and a V_{L} comprising a sequence set forth in SEQ ID NO: 5;
(ccix) a V_{H} comprising CDRs 1, 2 and 3 of a sequence set forth in SEQ ID NO: 115 and a V_{L} comprising CDRs 1, 2 and 3 of a sequence set forth in SEQ ID NO: 5;
(ccx) a V_{H} comprising a sequence set forth in SEQ ID NO: 115 and a V_{L} comprising a sequence set forth in SEQ ID NO: 5;
(ccxi) a V_{H} comprising CDRs 1, 2 and 3 of a sequence set forth in SEQ ID NO: 116 and a V_{L} comprising CDRs 1, 2 and 3 of a sequence set forth in SEQ ID NO: 5; or
(ccxii) a V_{H} comprising a sequence set forth in SEQ ID NO: 116 and a V_{L} comprising a sequence set forth in SEQ ID NO: 5.

In one example, the compound is an antibody comprising:
(i) a V_{H} comprising CDRs 1, 2 and 3 of a sequence set forth in SEQ ID NO: 64 and a V_{L} comprising CDRs 1, 2 and 3 of a sequence set forth in SEQ ID NO: 5; or
(ii) a V_{H} comprising a sequence set forth in SEQ ID NO: 64 and a V_{L} comprising a sequence set forth in SEQ ID NO: 5.

In one example, the compound is an antibody comprising a V_{H} comprising CDRs 1, 2 and 3 of a sequence set forth in SEQ ID NO: 64 and a V_{L} comprising CDRs 1, 2 and 3 of a sequence set forth in SEQ ID NO: 5.

In one example, the compound is an antibody comprising a V_{H} comprising a sequence set forth in SEQ ID NO: 64 and a V_{L} comprising a sequence set forth in SEQ ID NO: 5.

In one example, the CD131-binding protein or antibody as described herein comprises a human constant region, e.g., an IgG constant region, such as an IgG1, IgG2, IgG3 or IgG4 constant region or mixtures thereof. In the case of an antibody or protein comprising a V_{H} and a V_{L}, the V_{H} can be linked to a heavy chain constant region and the V_{L} can be linked to a light chain constant region.

The C-terminal lysine of the heavy chain constant region of a whole antibody (or a CD131-binding protein comprising a constant region or a C_{H}3) of the disclosure may be removed, for example, during production or purification of the antibody or protein, or by recombinantly engineering the nucleic acid encoding a heavy chain of the antibody. Accordingly, whole antibodies (or CD131-binding proteins) may comprise populations with all C-terminal lysine residues removed, populations with no C-terminal lysine residues removed, and/or populations having a mixture of protein with and without the C-terminal lysine residue. In some examples, the populations may additionally comprise protein in which the C-terminal lysine residue is removed in one of the heavy chain constant regions. Similarly, a composition of whole antibodies may comprise the same or a similar mix of antibody populations with or without the C-terminal lysine residue.

In one example, a protein or antibody as described herein comprises a constant region of an IgG4 antibody or a stabilized constant region of an IgG4 antibody. In one example, the protein or antibody comprises an IgG4 constant region with a proline at position 241 (according to the numbering system of Kabat (Kabat et al., Sequences of Proteins of Immunological Interest Washington DC United States Department of Health and Human Services, 1987 and/or 1991)).

In one example, the heavy chain constant region comprises a sequence set forth in SEQ ID NO: 197. In one example a protein or antibody as described herein or a composition of a protein or antibody as described herein, comprises a heavy chain constant region, including a stabilized heavy chain constant region, comprising a mixture of sequences fully or partially with or without the C-terminal lysine residue.

In one example, an antibody of the disclosure comprises a V_{H} disclosed herein linked or fused to an IgG4 constant region or stabilized IgG4 constant region (e.g., as discussed above) and the V_{L} is linked to or fused to a kappa light chain constant region.

The functional characteristics of a compound that binds CD131 (i.e., a CD131-binding protein) of the disclosure will be taken to apply mutatis mutandis to an antibody of the disclosure.

In one example, the compound that binds to CD131 is within a composition. For example, the composition comprises a compound (e.g., CD131-binding protein or antibody) as described herein. In one example, the composition additionally comprises one or more variants of the compound (i.e., protein or antibody). For example, that comprises a variant missing an encoded C-terminal lysine residue, a deamidated variant and/or a glycosylated variant and/or a variant comprising a pyroglutamate, e.g., at the N-terminus of a protein and/or a variant lacking a N-terminal residue, e.g., a N-terminal glutamine in an antibody or V region and/or a variant comprising all or part of a secretion signal. Deamidated variants of encoded asparagine residues may result in isoaspartic, and aspartic acid isoforms being generated or even a succinamide involving an adjacent amino acid residue. Deamidated variants of encoded glutamine residues may result in glutamic acid. Compositions comprising a heterogeneous mixture of such sequences and variants are intended to be included when reference is made to a particular amino acid sequence.

In one example, eosinophil and/or neutrophil levels are measured in the subject. For example, eosinophil and/or neutrophil levels are measured in the subject's sputum. In another example, eosinophil and/or neutrophil levels are measured in the subject's blood.

In one example, a method described herein further comprises determining a peripheral blood eosinophil and/or neutrophil count and/or a peripheral-blood differential eosinophil and/or neutrophil count in the subject and/or proportion of eosinophils and/or neutrophils in the subject's sputum. For example, the method further comprises determining a peripheral blood eosinophil and/or neutrophil count in the subject. In another example, the method further comprises determining a peripheral-blood differential eosinophil and/or neutrophil count in the subject. In a further example, the method further comprises determining a proportion of eosinophils and/or neutrophils in the subject's sputum.

In one example, the subject has a high sputum and/or high blood eosinophil and/or neutrophil count.

In one example, the subject has a high sputum and/or high blood eosinophil count. For example, the subject has an absolute eosinophil count of at least 500 cells per microliter of blood and/or the proportion of eosinophils in the subject's sputum is at least 3.00%. In one example, the subject has an absolute eosinophil count of at least 500 cells per microliter of blood. In one example, the proportion of eosinophils in the subject's sputum is at least 3.00%.

In one example, the subject has a high sputum and/or high blood neutrophil count. In one example, the subject has an absolute neutrophil count of at least 11,000 cells per microliter of blood and/or the proportion of neutrophils in the subject's sputum is at least 61.00%. In one example, the subject has an absolute neutrophil count of at least 11,000 cells per microliter of blood. In another example, the proportion of neutrophils in the subject's sputum is at least 61.00%.

In one example, the subject has a high sputum and/or high blood eosinophil and neutrophil count. For example, the subject has an absolute eosinophil count of at least 500 cells per microliter of blood and an absolute neutrophil count of at least 11,000 cells per microliter of blood, and/or the proportion of eosinophils in the subject's sputum is at least 3.00% and the proportion of neutrophils in the subject's sputum is at least 61.00%.

In one example, the subject is a human. In one example, the subject is an adult, for example over 18 years of age. In one example, the subject is a child, for example less than 18 years of age. In one example, the subject is 12 years of age or older. In one example, the subject is between 5 and 35 years of age. In one example, the subject is between 12 and 35 years of age. In one example, the subject is between 18 and 35 years of age.

### Key to Sequence Listing

SEQ ID NO 1: amino acid sequence of Homo sapiens CD131
SEQ ID NO 2: amino acid sequence of Homo sapiens CD131
SEQ ID NO 3: amino acid sequence of Homo sapiens GCS-F receptor
SEQ ID NO 4: amino acid sequence of Homo sapiens IL-5 receptor
SEQ ID NO 5: amino acid sequence of V_{L} chain of antibody 9A2
SEQ ID NO 6: amino acid sequence of V_{L} chain of antibody 9A2-VR1
SEQ ID NO 7: amino acid sequence of V_{L} chain of antibody 9A2-VR2
SEQ ID NO 8: amino acid sequence of V_{L} chain of antibody 9A2-VR3
SEQ ID NO 9: amino acid sequence of V_{L} chain of antibody 9A2-VR4
SEQ ID NO 10: amino acid sequence of V_{L} chain of antibody 9A2-VR5
SEQ ID NO 11: amino acid sequence of V_{L} chain of antibody 9A2-VR6
SEQ ID NO 12: amino acid sequence of V_{L} chain of antibody 9A2-VR8
SEQ ID NO 13: amino acid sequence of V_{L} chain of antibody 9A2-VR9
SEQ ID NO 14: amino acid sequence of V_{L} chain of antibody 9A2-VR11
SEQ ID NO 15: amino acid sequence of V_{L} chain of antibody 9A2-VR12
SEQ ID NO 16: amino acid sequence of V_{L} chain of antibody 9A2-VR13
SEQ ID NO 17: amino acid sequence of V_{L} chain of antibody 9A2-VR14
SEQ ID NO 18: amino acid sequence of V_{L} chain of antibody 9A2-VR16
SEQ ID NO 19: amino acid sequence of V_{L} chain of antibody 9A2-VR19
SEQ ID NO 20: amino acid sequence of V_{H} chain of antibody 9A2
SEQ ID NO 21: amino acid sequence of V_{H} chain of antibody 9A2-VR20
SEQ ID NO 22: amino acid sequence of V_{H} chain of antibody 9A2-VR21
SEQ ID NO 23: amino acid sequence of V_{H} chain of antibody 9A2-VR22
SEQ ID NO 24: amino acid sequence of V_{H} chain of antibody 9A2-VR23
SEQ ID NO 25: amino acid sequence of V_{H} chain of antibody 9A2-VR24
SEQ ID NO 26: amino acid sequence of V_{H} chain of antibody 9A2-VR26
SEQ ID NO 27: amino acid sequence of V_{H} chain of antibody 9A2-VR27
SEQ ID NO 28: amino acid sequence of V_{H} chain of antibody 9A2-VR28
SEQ ID NO 29: amino acid sequence of V_{H} chain of antibody 9A2-VR31
SEQ ID NO 30: amino acid sequence of V_{H} chain of antibody 9A2-VR32
SEQ ID NO 31: amino acid sequence of V_{H} chain of antibody 9A2-VR33
SEQ ID NO 32: amino acid sequence of V_{H} chain of antibody 9A2-VR34
SEQ ID NO 33: amino acid sequence of V_{H} chain of antibody 9A2-VR35
SEQ ID NO 34: amino acid sequence of V_{H} chain of antibody 9A2-VR36
SEQ ID NO 35: amino acid sequence of V_{H} chain of antibody 9A2-VR37
SEQ ID NO 36: amino acid sequence of V_{H} chain of antibody 9A2-VR38
SEQ ID NO 37: amino acid sequence of V_{H} chain of antibody 9A2-VR39
SEQ ID NO 38: amino acid sequence of V_{H} chain of antibody 9A2-VR40
SEQ ID NO 39: amino acid sequence of V_{H} chain of antibody 9A2-VR41
SEQ ID NO 40: amino acid sequence of V_{H} chain of antibody 9A2-VR42
SEQ ID NO 41: amino acid sequence of V_{H} chain of antibody 9A2-VR43
SEQ ID NO 42: amino acid sequence of V_{H} chain of antibody 9A2-VR44
SEQ ID NO 43: amino acid sequence of V_{H} chain of antibody 9A2-VR45
SEQ ID NO 44: amino acid sequence of V_{H} chain of antibody 9A2-VR46
SEQ ID NO 45: amino acid sequence of V_{H} chain of antibody 9A2-VR47
SEQ ID NO 46: amino acid sequence of V_{H} chain of antibody 9A2-VR48
SEQ ID NO 47: amino acid sequence of V_{H} chain of antibody 9A2-VR49
SEQ ID NO 48: amino acid sequence of V_{H} chain of antibody 9A2-VR50
SEQ ID NO 49: amino acid sequence of V_{H} chain of antibody 9A2-VR24.04
SEQ ID NO 50: amino acid sequence of V_{H} chain of antibody 9A2-VR24.07
SEQ ID NO 51: amino acid sequence of V_{H} chain of antibody 9A2-VR24.10
SEQ ID NO 52: amino acid sequence of V_{H} chain of antibody 9A2-VR24.12
SEQ ID NO 53: amino acid sequence of V_{H} chain of antibody 9A2-VR24.19
SEQ ID NO 54: amino acid sequence of V_{H} chain of antibody 9A2-VR24.24
SEQ ID NO 55: amino acid sequence of V_{H} chain of antibody 9A2-VR24.76
SEQ ID NO 56: amino acid sequence of V_{H} chain of antibody 9A2-VR24.78
SEQ ID NO 57: amino acid sequence of V_{H} chain of antibody 9A2-VR24.81
SEQ ID NO 58: amino acid sequence of V_{H} chain of antibody 9A2-VR24.82
SEQ ID NO 59: amino acid sequence of V_{H} chain of antibody 9A2-VR24.84
SEQ ID NO 60: amino acid sequence of V_{H} chain of antibody 9A2-VR24.87
SEQ ID NO 61: amino acid sequence of V_{H} chain of antibody 9A2-VR24.91
SEQ ID NO 62: amino acid sequence of V_{H} chain of antibody 9A2-VR24.93
SEQ ID NO 63: amino acid sequence of V_{H} chain of antibody 9A2-VR24.27
SEQ ID NO 64: amino acid sequence of V_{H} chain of antibody 9A2-VR24.29
SEQ ID NO 65: amino acid sequence of V_{H} chain of antibody 9A2-VR24.30
SEQ ID NO 66: amino acid sequence of V_{H} chain of antibody 9A2-VR24.33
SEQ ID NO 67: amino acid sequence of V_{H} chain of antibody 9A2-VR24.44
SEQ ID NO 68: amino acid sequence of V_{H} chain of antibody 9A2-VR24.97
SEQ ID NO 69: amino acid sequence of V_{H} chain of antibody 9A2-VR24.98
SEQ ID NO 70: amino acid sequence of V_{H} chain of antibody 9A2-VR24.102
SEQ ID NO 71: amino acid sequence of V_{H} chain of antibody 9A2-VR24.107
SEQ ID NO 72: amino acid sequence of V_{H} chain of antibody 9A2-VR24.110
SEQ ID NO 73: amino acid sequence of V_{H} chain of antibody 9A2-VR24.111
SEQ ID NO 74: amino acid sequence of V_{H} chain of antibody 9A2-VR24.55
SEQ ID NO 75: amino acid sequence of V_{H} chain of antibody 9A2-VR24.56
SEQ ID NO 76: amino acid sequence of V_{H} chain of antibody 9A2-VR24.57
SEQ ID NO 77: amino acid sequence of V_{H} chain of antibody 9A2-VR24.122
SEQ ID NO 78: amino acid sequence of V_{H} chain of antibody 9A2-VR24.124
SEQ ID NO 79: amino acid sequence of V_{H} chain of antibody 9A2-VR24.131
SEQ ID NO 80: amino acid sequence of V_{H} chain of antibody 9A2-VR39.01
SEQ ID NO 81: amino acid sequence of V_{H} chain of antibody 9A2-VR39.02
SEQ ID NO 82: amino acid sequence of V_{H} chain of antibody 9A2-VR39.04
SEQ ID NO 83: amino acid sequence of V_{H} chain of antibody 9A2-VR39.05
SEQ ID NO 84: amino acid sequence of V_{H} chain of antibody 9A2-VR39.06
SEQ ID NO 85: amino acid sequence of V_{H} chain of antibody 9A2-VR39.11
SEQ ID NO 86: amino acid sequence of V_{H} chain of antibody 9A2-VR39.12
SEQ ID NO 87: amino acid sequence of V_{H} chain of antibody 9A2-VR39.16
SEQ ID NO 88: amino acid sequence of V_{H} chain of antibody 9A2-VR39.17
SEQ ID NO 89: amino acid sequence of V_{H} chain of antibody 9A2-VR39.18
SEQ ID NO 90: amino acid sequence of V_{H} chain of antibody 9A2-VR39.19
SEQ ID NO 91: amino acid sequence of V_{H} chain of antibody 9A2-VR39.21
SEQ ID NO 92: amino acid sequence of V_{H} chain of antibody 9A2-VR39.22
SEQ ID NO 93: amino acid sequence of V_{H} chain of antibody 9A2-VR39.23
SEQ ID NO 94: amino acid sequence of V_{H} chain of antibody 9A2-VR39.24
SEQ ID NO 95: amino acid sequence of V_{H} chain of antibody 9A2-VR39.97
SEQ ID NO 96: amino acid sequence of V_{H} chain of antibody 9A2-VR39.98
SEQ ID NO 97: amino acid sequence of V_{H} chain of antibody 9A2-VR39.102
SEQ ID NO 98: amino acid sequence of V_{H} chain of antibody 9A2-VR39.103
SEQ ID NO 99: amino acid sequence of V_{H} chain of antibody 9A2-VR39.105
SEQ ID NO 100: amino acid sequence of V_{H} chain of antibody 9A2-VR39.109
SEQ ID NO 101: amino acid sequence of V_{H} chain of antibody 9A2-VR39.110
SEQ ID NO 102: amino acid sequence of V_{H} chain of antibody 9A2-VR39.111
SEQ ID NO 103: amino acid sequence of V_{H} chain of antibody 9A2-VR39.112
SEQ ID NO 104: amino acid sequence of V_{H} chain of antibody 9A2-VR39.116
SEQ ID NO 105: amino acid sequence of V_{H} chain of antibody 9A2-VR39.27
SEQ ID NO 106: amino acid sequence of V_{H} chain of antibody 9A2-VR39.28
SEQ ID NO 107: amino acid sequence of V_{H} chain of antibody 9A2-VR39.46
SEQ ID NO 108: amino acid sequence of V_{H} chain of antibody 9A2-VR39.122
SEQ ID NO 109: amino acid sequence of V_{H} chain of antibody 9A2-VR39.139
SEQ ID NO 110: amino acid sequence of V_{H} chain of antibody 9A2-VR39.140
SEQ ID NO 111: amino acid sequence of V_{H} chain of antibody 9A2-VR39.148
SEQ ID NO 112: amino acid sequence of V_{H} chain of antibody 9A2-VR39.162
SEQ ID NO 113: amino acid sequence of V_{H} chain of antibody 9A2-VR39.77
SEQ ID NO 114: amino acid sequence of V_{H} chain of antibody 9A2-VR39.93
SEQ ID NO 115: amino acid sequence of V_{H} chain of antibody 9A2-VR39.174
SEQ ID NO 116: amino acid sequence of V_{H} chain of antibody 9A2-VR39.177
SEQ ID NO 117: amino acid sequence of soluble Homo sapiens CD131 comprising a C-terminal 6×His tag and comprising the substitution N37A
SEQ ID NO 118: amino acid sequence of soluble Homo sapiens CD131 comprising a C-terminal 6×His tag and comprising the substitution D38A
SEQ ID NO 119: amino acid sequence of soluble Homo sapiens CD131 comprising a C-terminal 6×His tag and comprising the substitution Y39A
SEQ ID NO 120: amino acid sequence of soluble Homo sapiens CD131 comprising a C-terminal 6×His tag and comprising the substitution T40A
SEQ ID NO 121: amino acid sequence of soluble Homo sapiens CD131 comprising a C-terminal 6×His tag and comprising the substitution S41A
SEQ ID NO 122: amino acid sequence of soluble Homo sapiens CD131 comprising a C-terminal 6×His tag and comprising the substitution H42A
SEQ ID NO 123: amino acid sequence of soluble Homo sapiens CD131 comprising a C-terminal 6×His tag and comprising the substitution S102A
SEQ ID NO 124: amino acid sequence of soluble Homo sapiens CD131 comprising a C-terminal 6×His tag and comprising the substitution F103A
SEQ ID NO 125: amino acid sequence of soluble Homo sapiens CD131 comprising a C-terminal 6×His tag and comprising the substitution V104A
SEQ ID NO 126: amino acid sequence of soluble Homo sapiens CD131 comprising a C-terminal 6×His tag and comprising the substitution V105A
SEQ ID NO 127: amino acid sequence of soluble Homo sapiens CD131 comprising a C-terminal 6×His tag and comprising the substitution T106A
SEQ ID NO 128: amino acid sequence of soluble Homo sapiens CD131 comprising a C-terminal 6×His tag and comprising the substitution D107A
SEQ ID NO 129: amino acid sequence of soluble Homo sapiens CD131 comprising a C-terminal 6×His tag and comprising the substitution V108A
SEQ ID NO 130: amino acid sequence of soluble Homo sapiens CD131 comprising a C-terminal 6×His tag and comprising the substitution N337A
SEQ ID NO 131: amino acid sequence of soluble Homo sapiens CD131 comprising a C-terminal 6×His tag and comprising the substitution I338A
SEQ ID NO 132: amino acid sequence of soluble Homo sapiens CD131 comprising a C-terminal 6×His tag and comprising the substitution Q339A
SEQ ID NO 133: amino acid sequence of soluble Homo sapiens CD131 comprising a C-terminal 6×His tag and comprising the substitution M340A
SEQ ID NO 134: amino acid sequence of soluble Homo sapiens CD131 comprising a C-terminal 6×His tag and comprising the substitution K362A
SEQ ID NO 135: amino acid sequence of soluble Homo sapiens CD131 comprising a C-terminal 6×His tag and comprising the substitution M363A
SEQ ID NO 136: amino acid sequence of soluble Homo sapiens CD131 comprising a C-terminal 6×His tag and comprising the substitution R364A
SEQ ID NO 137: amino acid sequence of soluble Homo sapiens CD131 comprising a C-terminal 6×His tag and comprising the substitution Y365A
SEQ ID NO 138: amino acid sequence of soluble Homo sapiens CD131 comprising a C-terminal 6×His tag and comprising the substitution E366A
SEQ ID NO 139: amino acid sequence of soluble Homo sapiens CD131 comprising a C-terminal 6×His tag and comprising the substitution H367A
SEQ ID NO 140: amino acid sequence of soluble Homo sapiens CD131 comprising a C-terminal 6×His tag and comprising the substitution I368A
SEQ ID NO 141: amino acid sequence of soluble Homo sapiens CD131 comprising a C-terminal 6×His tag and comprising the substitution D369A
SEQ ID NO 142: amino acid sequence of soluble Homo sapiens CD131 comprising a C-terminal 6×His tag and comprising the substitution R418A
SEQ ID NO 143: amino acid sequence of soluble Homo sapiens CD131 comprising a C-terminal 6×His tag and comprising the substitution T419A
SEQ ID NO 144: amino acid sequence of soluble Homo sapiens CD131 comprising a C-terminal 6×His tag and comprising the substitution n G420A
SEQ ID NO 145: amino acid sequence of soluble Homo sapiens CD131 comprising a C-terminal 6×His tag and comprising the substitution Y421A
SEQ ID NO 146: amino acid sequence of soluble Homo sapiens CD131 comprising a C-terminal 6×His tag and comprising the substitution N422A
SEQ ID NO 147: amino acid sequence of soluble Homo sapiens CD131 comprising a C-terminal 6×His tag and comprising the substitution G423A
SEQ ID NO 148: amino acid sequence of soluble Homo sapiens CD131 comprising a C-terminal 6×His tag and comprising the substitution I424A
SEQ ID NO 149: nucleotide acid sequence encoding trimer oligonucleotide 9A2 L1.1
SEQ ID NO 150: nucleotide acid sequence encoding trimer oligonucleotide 9A2 L3.1
SEQ ID NO 151: nucleotide acid sequence encoding trimer oligonucleotide 9A2 L3.2
SEQ ID NO 152: nucleotide acid sequence encoding trimer oligonucleotide 9A2 H1.1
SEQ ID NO 153: nucleotide acid sequence encoding trimer oligonucleotide 9A2 H2.1
SEQ ID NO 154: nucleotide acid sequence encoding trimer oligonucleotide 9A2 H3.1
SEQ ID NO 155: nucleotide acid sequence encoding trimer oligonucleotide 9A2 H3.2
SEQ ID NO 156: nucleotide acid sequence encoding trimer oligonucleotide 9A2-VR24-H2.1
SEQ ID NO 157: nucleotide acid sequence encoding trimer oligonucleotide 9A2-VR24-H2.2
SEQ ID NO 158: nucleotide acid sequence encoding trimer oligonucleotide 9A2-VR24-H2.3
SEQ ID NO 159: nucleotide acid sequence encoding trimer oligonucleotide 9A2-VR39-H1.1
SEQ ID NO 160: nucleotide acid sequence encoding trimer oligonucleotide 9A2-VR39-H1.2
SEQ ID NO 161: nucleotide acid sequence encoding trimer oligonucleotide 9A2-VR39-H2.2
SEQ ID NO 162: nucleotide acid sequence encoding trimer oligonucleotide 9A2-VR39-H2.3
SEQ ID NO 163: amino acid sequence of V_{H} chain of stop template of 9A2 H1.1
SEQ ID NO 164: amino acid sequence of V_{H} chain of stop template of 9A2 H2.1
SEQ ID NO 165: amino acid sequence of V_{H} chain of stop template of 9A2 H3.1
SEQ ID NO 166: amino acid sequence of V_{H} chain of stop template of 9A2 H3.2
SEQ ID NO 167: amino acid sequence of V_{L} chain of stop template of 9A2 L1.1
SEQ ID NO 168: amino acid sequence of V_{L} chain of stop template of 9A2 L3.1
SEQ ID NO 169: amino acid sequence of V_{L} chain of stop template of 9A2 L3.2
SEQ ID NO 170: amino acid sequence of V_{H} chain of stop template of 9A2 VR24-H2.1
SEQ ID NO 171: amino acid sequence of V_{H} chain of stop template of 9A2 VR24-H2.2
SEQ ID NO 172: amino acid sequence of V_{H} chain of stop template of 9A2 VR24-H2.3
SEQ ID NO 173: amino acid sequence of V_{H} chain of stop template of 9A2 VR39-H1.1
SEQ ID NO 174: amino acid sequence of V_{H} chain of stop template of 9A2 VR39-H1.2
SEQ ID NO 175: amino acid sequence of V_{H} chain of stop template of 9A2 VR39-H2.2
SEQ ID NO 176: amino acid sequence of V_{H} chain of stop template of 9A2 VR39-H2.3
SEQ ID NO 177: amino acid sequence of consensus of V_{L} chain of 9A2 and derivatives
SEQ ID NO 178: amino acid sequence of consensus of CDR1 of V_{L} chain of 9A2 and derivatives
SEQ ID NO 179: amino acid sequence of consensus of CDR3 of V_{L} chain of 9A2 and derivatives
SEQ ID NO 180: amino acid sequence of consensus of V_{H} chain of 9A2 and derivatives
SEQ ID NO 181: amino acid sequence of consensus of CDR1 of V_{H} chain of 9A2 and derivatives
SEQ ID NO 182: amino acid sequence of consensus of CDR2 of V_{H} chain of 9A2 and derivatives
SEQ ID NO 183: amino acid sequence of consensus of CDR3 of V_{H} chain of 9A2 and derivatives
SEQ ID NO 184: amino acid sequence of consensus of V_{H} chain of 9A2-VR24 and derivatives
SEQ ID NO 185: amino acid sequence of consensus of CDR1 of V_{H} chain of 9A2-VR24 and derivatives
SEQ ID NO 186: amino acid sequence of consensus of CDR2 of V_{H} chain of 9A2-VR24 and derivatives
SEQ ID NO 187: amino acid sequence of consensus of CDR3 of V_{H} chain of 9A2-VR24 and derivatives
SEQ ID NO 188: amino acid sequence of consensus of V_{H} chain of 9A2-VR39 and derivatives
SEQ ID NO 189: amino acid sequence of consensus of CDR1 of V_{H} chain of 9A2-VR39 and derivatives
SEQ ID NO 190: amino acid sequence of consensus of CDR2 of V_{H} chain of 9A2-VR39 and derivatives
SEQ ID NO 191: amino acid sequence of consensus of CDR3 of V_{H} chain of 9A2-VR39 and derivatives
SEQ ID NO: 192: amino acid sequence of soluble Homo sapiens CD131 comprising a C-terminal 6×His tag
SEQ ID NO: 193: amino acid sequence of V_{H} chain of 9A2-VR24 HCDR2 mutants
SEQ ID NO: 194: amino acid sequence of antibody 9A2 heavy chain
SEQ ID NO: 195: amino acid sequence of antibody 9A2 light chain
SEQ ID NO: 196: amino acid sequence of stabilized IgG4 heavy chain constant region
SEQ ID NO: 197: amino acid sequence of kappa light chain constant region

### BRIEF DESCRIPTION OF THE DRAWINGS

FIG. 1 is a series of graphical representations showing expression of (A) CSF2RB, (B) GMCSF and (C) IL-5 in severe asthmatics. (D) Cluster analysis of CSF2RB expression in neutrophilic asthma relative to non-neutrophilic asthma and (E) correlation with BAL neutrophil percentage. CSF2RB expression in patients with (F) bacterial detection irrespective of (G) atopy status. (H) COL1A1 expression in patients with severe asthma relative to mild-to-moderate asthma. Expression was positively correlated with patients' (I) ACQ scores, and (J) CSF2RB expression. Analysis includes non-parametric Mann Whitney T test and Spearman's correlation and data are expressed as median±interquartile ranges; *p<0.05, **p<0.01, ***p<0.001, ****p<0.0001.

FIG. 2 is a series of graphical representations showing CFA-HDM treated hβcTg mice demonstrate a mixed granulocytic lung inflammation. (A) Schematic representation of treatment protocol in hβcTg mice. White blood cell analysis including (B) blood monocytes and (C) blood neutrophils in mice treated with CFA-HDM. BAL cell analysis including (D) total BAL cell number, (E) BAL macrophages, (F) BAL neutrophils and (G) BAL eosinophils in mice treated with CFA-HDM. Flow cytometry analysis in lung tissue including (H) total lung leukocytes, (I) lung neutrophils, (J) lung eosinophils, (K) lung exudative macrophages, (L) alveolar macrophages and (M) lung monocytes in mice treated with CFA-HDM. n=6; data are mean±SEM; *p<0.05, **p<0.01, ***p<0.001 by unpaired student's T tests.

FIG. 3 is a series of graphical representations showing CFA-HDM treated hβcTg mice develop AHR and baseline airway restriction. (A) Dose-response curves of respiratory system resistance (Rrs) and (B) maximum Rrs. (C) Dose-response curves of conducting airway resistance (Rn) and (D) maximum Rn. (E) Baseline Pressure-Volume (P-V) curve, including (F) statistic compliance (Cst) and (G) respiratory system elastance (Ers). n=6; data are mean±SEM; *p<0.05, **p<0.01, ***p<0.001 by unpaired student's T tests, or repeated student's T tests.

FIG. 4 is a series of graphical representations showing CFA-HDM treated hβcTg mice develop pulmonary inflammation, airway fibrosis, edema and NETosis. (A) Inflammation score and (B) collagen-positive area around the airways. (C) RT-qPCR analysis on Col1a1. (D) Total protein and (E) LDH in BALF. Levels of (F) dsDNA and (G) MPO activity in BALF. RT-qPCR analysis on lung (H) CSF2RB, (I) Il-5, (J) Il-3 and (K) Gm-csf in CFA-HDM treated mice. (L) Pearson's correlation analysis between the gene expression of Col1a1 and CSF2RB using dCT values. n=6; data are mean±SEM; *p<0.05, **p<0.01, ***p<0.001 by unpaired student's T tests.

FIG. 5 is a series of graphical representations showing CSL311 reduces mixed granulocytic inflammation. (A) Schematic representation of treatment protocol. White blood cell analysis of (B) blood monocytes and blood neutrophils in CFA-HDM, CFA-HDM/ISO and CFA-HDM/CSL311 treated mice. (C) BAL fluid cell analysis of BAL macrophages, BAL neutrophils and BAL eosinophils in CFA-HDM, CFA-HDM/ISO and CFA-HDM/CSL311 treated mice. (D) Flow cytometry analysis in lung tissue of lung neutrophils, lung eosinophils, alveolar macrophages and interstitial macrophages in CFA-HDM, CFA-HDM/ISO and CFA-HDM/CSL311 treated mice. (E) Total protein and levels of dsDNA in BALF in CFA-HDM, CFA-HDM/ISO and CFA-HDM/CSL311 treated mice. (F) MPO activity and LDH in BALF in CFA-HDM, CFA-HDM/ISO and CFA-HDM/CSL311 treated mice. (G) RT-qPCR analysis on lung CSF2RB, Il4, Il-13, Il-17, Ifng, Il12b and Ccl17 in CFA-HDM, CFA-HDM/ISO and CFA-HDM/CSL311 treated mice. n=9; data are mean±SEM; *p<0.05, **p<0.01, ***p<0.001 ISO vs. SAL and #n<0.05, ##p<0.01, ###p<0.001 ISO vs CSL311 by One-Way ANOVA.

FIG. 6 is a series of graphical representations showing CSL311 reduces AHR and baseline airway restriction. (A) Dose-response curves of respiratory system resistance (Rrs) and (B) maximum Rrs. (C) Dose-response curves of conducting airway resistance (Rn) and (D) maximum Rn. (E) Baseline Pressure-Volume (P-V) curve including (F) statistic compliance (Cst) and (G) respiratory system elastance (Ers) in CFA-HDM/ISO and CFA-HDM/CSL311 treated mice. n=9; data are mean±SEM; *p<0.05, **p<0.01, ***p<0.001 by One-Way ANOVA.

FIG. 7 is a series of graphical representations showing CSL311 reduces pulmonary inflammation and airway fibrosis. (A) Inflammation score and (B) collagen-positive area around the airways in CFA-HDM/ISO and CFA-HDM/CSL311 treated mice. (C) RT-qPCR analysis on Col1a1. n=9; data are mean±SEM; *p<0.05, **p<0.01, ***p<0.001 by One-Way ANOVA.

FIG. 8 is a series of graphical representations showing CSL311 reduces lung expression of CSF2RB and Th2/Th17 cytokines while preserving Th1 cytokines. Lung expression of (A) CSF2RB, (B) Il-5, (C) Il-3, (D) Gm-csf, Th2 cytokines (E) Il-4 and (F) Il-13, Th1 cytokines (G) Ifng and (H) Il-12, Th2 cell chemokine (I) Ccl-17, (J) neutrophil cytokine Il-17, neutrophil chemokine (K) Cxcl1 and macrophage chemokine (L) Ccl-2. n=9; data are mean±SEM; *p<0.05, **p<0.01, ***p<0.001 by One-Way ANOVA.

FIG. 9 is a series of graphical representations showing CSL311 improves airway function and airway hyperreactivity (AHR) in a house-dust mite (HDM) challenge model. (A) Treatment protocol in hβcTg mice and WT mice. (B) Compliance (CsT), (C) forced vital capacity (FVC) and (D) forced expiratory volume in 0.1 seconds (FEV0.1) 48 hours after challenge. (E) Lung function in HDM challenge model 48 hours after the last HDM challenge, * indicates p<0.05, 2-way ANOVA, significantly increased compared to hβcTg PBS and hβcTg Isotype group. (F) Collagen and smooth muscle thickness and (G) the proportion of collagen in airway tissue. n, WT HDM=4; hbcTg PBS=3, hbcTg HDM+ isotype control antibody=5; and hbcTg HDM+CSL311 antibody=6. Data are expressed as mean+SEM. * indicates p<0.05, Student's t test, significantly increased compared to PBS control group.

FIG. 10 is a series of graphical representations showing CSL311 improves airway function and reduces remodelling following chronic intranasal ASP challenge. (A) Treatment protocol schematic. Lung function in ASP challenge model 48 hours after the last HDM challenge including (B) system elastance (Ers), (C) Compliance (CsT); and (D) pressure volume curves. (E) Inflammation score. (F) Quantitation of collagen positive pixels from ASP+ isotype and ASP+CSL311 groups and (G) airway subepithelial wall thickness. n=9; data are mean±SEM; *p<0.05, **p<0.01, ***p<0.001 ISO vs. SAL and #p<0.05, ##p<0.01, ###p<0.001 ISO vs CSL311 by One-Way ANOVA.

FIG. 11 is a series of graphical representations showing blocking βc signaling inhibits inflammation following 8 weeks of intranasal Aspergillus extract exposure+/−CSL311 or isotype control including (A) multi dimensional scaling of all mice. Expression of genes involved in inflammation and immunity measured by RNA-sequencing including (B) Cd3d (C) Cd8a (D) Cd4 (E) Cd79a (F) Chil3 (G) Arg1 (H) Ear2 (I)I Prg2 (J) Cd86 (K) H2-Ab1 (L) Ccl11 (M) Ccl17. Expression of genes involved in tissue remodelling measured by RNA-sequencing including (N) Mmp12 (O) Mmp13 (P) Tgfb1 (Q) Thbs4 and (F) Fmod. (n=3 PBS, 5 ASP+Iso, 5 ASP+CSL311). * indicates p<0.05

FIG. 12 is a series of graphical representations showing effect of CSL311 treatment on (A) BALF cells (B) BALF macrophages (C) BALF neutrophils (D) BALF eosinophils (E) lung neutrophils (F) lung CD11b+ macrophages and (G) lung eosinophils in hβcTg mice exposed to HDM and PPE (ACO). A separate cohort of mice were challenged to inhaled methacholine (Mch) to measure total respiratory resistance (Rrs, H) and airway resistance (Rn, J), where maximal Mch responses were also presented (Rrs Max, I; Rn Max, K). Airspace enlargement (emphysema) was measured by the mean linear intercept method (Lm) on H&E stained lung sections (L). A schematic of the treatment protocol is depicted in M. n=6 for each group. *p<0.05, **p<0.01, ***p<0.001 One-way ANOVA with Tukey's post hoc analysis.

FIG. 13 is a series of graphical representations showing STAT5 phosphorylation in response to GM-CSF stimulation and subsequent blocking of activation by CSL311 in (A) monocytes (B) basophils (C) eosinophils and (D) neutrophils.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

For the purposes of nomenclature only and not limitation an exemplary sequence of a human CD131 (pre-CD131) is set out in NCBI Reference Sequence: NP_000386.1 and NCBI Genbank Accession Number P32927 (and set out in SEQ ID NO: 1). A sequence of a mature human CD131 lacks amino acids 1 to 16 of SEQ ID NO: 1. Positions of amino acids are often referred to herein by reference to pre-CD131. The positions in mature CD131 is readily determined by accounting for the signal sequence (amino acids 1-16 in the case of SEQ ID NO: 1). The sequence of CD131 from other species can be determined using sequences provided herein and/or in publicly available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)). Reference to human CD131 may be abbreviated to hCD131. Reference to soluble CD131 refers to polypeptides comprising the extracellular region of CD131, e.g., amino acids 17 to 438 of SEQ ID NO: 1.

Reference herein to CD131 includes native forms of CD131 and mutant forms thereof retaining an ability to bind to CD131 (e.g., hCD131) and induce signaling. CD131 is also known as “CSF2RB” and “cytokine receptor common subunit beta” and “β (beta) common receptor” (abbreviated as “βCR” or “βc”).

A “compound”, as contemplated by the present disclosure, can take any of a variety of forms including natural compounds, chemical small molecule compounds or biological compounds or macromolecules. Exemplary compounds include an antibody or a protein comprising an antigen binding fragment of an antibody, a nucleic acid, a polypeptide, a peptide, and a small molecule.

The terms “reduce” or “reducing” as used herein in reference to cells, e.g., eosinophils, refers to administering a compound described herein to stop or hinder the development, differentiation and/or maturation, accumulation, migration and/or recruitment of cells, e.g., eosinophils, in the subject.

The terms “deplete” or “depleting” as used herein in reference to eosinophils refers to administering a compound described herein to stop or hinder the survival of eosinophils in the subject. For example, the compound may induce eosinophil cell death by e.g., apoptosis (programmed cell death), cytolysis, autophagy and/or netosis. The compound may act by blocking pro-survival signals (e.g. IL-5 and/or GM-CSF-mediated signals) to cells thereby leading to cell death, e.g. by apoptosis.

In the context of asthma, the term “treating” or “treat” refers to administering a compound described herein to reduce, eliminate, or prevent an occurrence or exacerbation of at least one symptom. For example, a compound described herein can be administered in order to prevent an asthmatic attack. Alternatively, or additionally, the compound can be administered to alleviate asthmatic symptoms such as wheezing, shortness of breath, chest tightness, and/or coughing.

The terms “reduce” and “prevent an increase in” are used herein to refer to a lower amount of any of the genes and/or factors recited herein, relative to either the amount in the subject prior to administration of the compound that binds to CD131, or relative to the amount in a corresponding control subject. For instance, the control subject may be a subject who receives a placebo and/or a standard of care therapy, rather than the compound that binds to CD131.

The term “therapeutically effective amount” is at least the minimum concentration required to effect a measurable improvement of a particular disease or condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present disclosure to a specific quantity, e.g., weight or amount of protein(s), rather the present disclosure encompasses any amount of the CD131-binding compound(s) sufficient to achieve the stated result in a subject.

As used herein, the term “subject” shall be taken to mean any animal including humans, for example a mammal. Exemplary subjects include but are not limited to humans and non-human primates. In one example, the subject is a human.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions. In some examples, the protein is a fusion protein. As used herein, a “fusion protein” is a protein comprising at least two domains that have been joined so that they are translated as a single unit, producing a single protein.

The term “polypeptide” or “polypeptide chain” will be understood from the foregoing paragraph to mean a series of contiguous amino acids linked by peptide bonds.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

As used herein, the term “antigen binding site” shall be taken to mean a structure formed by a protein that is capable of binding or specifically binding to an antigen. The antigen binding site need not be a series of contiguous amino acids, or even amino acids in a single polypeptide chain. For example, in a Fv produced from two different polypeptide chains the antigen binding site is made up of a series of amino acids of a V_{L} and a V_{H} that interact with the antigen and that are generally, however not always in the one or more of the CDRs in each variable region. In some examples, an antigen binding site is or comprises a V_{H} or a V_{L} or a Fv. In some examples, the antigen binding site comprises one or more CDRs of an antibody. The antigen binding site need not be in the context of an entire antibody, e.g., it can be in isolation (e.g., a domain antibody) or in another form, e.g., as described herein, such as a scFv.

The skilled artisan will be aware that an “antibody” is generally considered to be a protein that comprises a variable region made up of a plurality of polypeptide chains, e.g., a polypeptide comprising a V_{L} and a polypeptide comprising a V_{H}. An antibody also generally comprises constant domains, some of which can be arranged into a constant region, which includes a constant fragment or fragment crystallizable (Fc), in the case of a heavy chain. A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies and chimeric antibodies.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs. In the case of a protein derived from an IgNAR, the protein may lack a CDR2. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are necessary for antigen binding. Each variable region typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol Biol. 196: 901-917, 1987; Chothia et al. Nature 342, 877-883, 1989; and/or Al-Lazikani et al., J Mol Biol 273: 927-948, 1997; the IMGT numbering system of Lefranc et al., Devel. And Compar. Immunol., 27: 55-77, 2003; or the AHO numbering system of Honnegher and Plükthun J. Mol. Biol., 309: 657-670, 2001. For example, according to the numbering system of Kabat, V_{H} framework regions (FRs) and CDRs are positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4). The present disclosure is not limited to FRs and CDRs as defined by the Kabat numbering system, but includes all numbering systems, including those discussed above. In one example, reference herein to a CDR (or a FR) is in respect of those regions according to the Kabat numbering system.

“Framework regions” (FRs) are those variable region residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)₂ fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of a compound or an antigen binding site thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the protein, will reduce the amount of labeled “A” bound to the antibody.

As used herein, the term “specifically binds” or “binds specifically” shall be taken to mean that a compound of the disclosure reacts or associates more frequently, more rapidly, with greater duration and/or with greater affinity with a particular antigen or cell expressing same than it does with alternative antigens or cells. For example, a compound binds to CD131 (e.g., hCD131 or a polypeptide comprising a region thereof, e.g., a polypeptide comprising a sequence set forth in SEQ ID NO: 191) with materially greater affinity (e.g., 20 fold or 40 fold or 60 fold or 80 fold to 100 fold or 150 fold or 200 fold) than it does to other cytokine receptors or to antigens commonly recognized by polyreactive natural antibodies (i.e., by naturally occurring antibodies known to bind a variety of antigens naturally found in humans). In an example of the present disclosure, a compound “specifically binds” to one form of hCD131 or a polypeptide comprising a region thereof (e.g., the extracellular region of hCD131) or a polypeptide comprising a sequence set forth in SEQ ID NO: 191 with an affinity at least 1.5 fold or 2 fold or greater (e.g., 5 fold or 10 fold or 20 fold r 50 fold or 100 fold or 200 fold) than it does to a mutant form of SEQ ID NO: 191 comprising a sequence set forth in SEQ ID NO: 119, 124, 131 or 137. Reference to “binding” provides explicit support for the term “specific binding” and vice versa.

A protein or antibody may be considered to “preferentially bind” to a polypeptide if it binds that polypeptide with a dissociation constant (K_{D}) that is less than the protein's or antibody's K_{D} for another polypeptide. In one example, a protein or antibody is considered to preferentially bind to a polypeptide if it binds the polypeptide with an affinity (i.e., K_{D}) that is at least about 20 fold or 40 fold or 60 fold or 80 fold or 100 fold or 120 fold or 140 fold or 160 fold more than the protein's or antibody's K_{D} for another polypeptide.

For the purposes of clarification and as will be apparent to the skilled artisan based on the exemplified subject matter herein, reference to “affinity” in this specification is a reference to K_{D} of a protein or antibody.

For the purposes of clarification and as will be apparent to the skilled artisan based on the description herein, reference to “a K_{D} of X nM or less” will be understood to mean that the numerical value of the K_{D} is equal to X nM or is lower in numerical value. As a skilled person would understand a lower numerical value of a K_{D} corresponds to a higher (i.e., stronger) affinity, i.e., an affinity of 2 nM is stronger than an affinity of 3 nM.

An “IC₅₀ of at least about” will be understood to mean that the IC₅₀ is equal to the recited value or greater (i.e., the numerical value recited as the IC₅₀ is lower), i.e., an IC₅₀ of 2 nM is greater than an IC₅₀ of 3 nM. Stated another way, this term could be “an IC₅₀ of X or less”, wherein X is a value recited herein.

As used herein, the term “epitope” (syn. “antigenic determinant”) shall be understood to mean a region of CD131 to which a compound comprising an antigen binding site of an antibody binds. This term is not necessarily limited to the specific residues or structure to which the compound makes contact. For example, this term includes the region spanning amino acids contacted by the compound and/or 5-10 or 2-5 or 1-3 amino acids outside of this region. In some examples, the epitope comprises a series of discontinuous amino acids that are positioned close to one another when CD131 is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

The term “competitively inhibits” shall be understood to mean that a compound (i.e., CD131-binding protein or antibody) of the disclosure (or an antigen binding site thereof) reduces or prevents binding of a recited antibody or protein to CD131. This may be due to the protein (or antigen binding site) and antibody binding to the same or an overlapping epitope. It will be apparent from the foregoing that the compound need not completely inhibit binding of the antibody, rather it need only reduce binding by a statistically significant amount, for example, by at least about 10% or 20% or 30% or 40% or 50% or 60% or 70% or 80% or 90% or 95%. Preferably, the compound reduces binding of the protein or antibody to CD131 by at least about 30%, more preferably by at least about 50%, more preferably, by at least about 70%, still more preferably by at least about 75%, even more preferably, by at least about 80% or 85% and even more preferably, by at least about 90%. Methods for determining competitive inhibition of binding are known in the art and/or described herein. For example, the antibody is exposed to CD131 either in the presence or absence of the compound. If less antibody binds in the presence of the compound than in the absence of the compound, the compound is considered to competitively inhibit binding of the antibody. In one example, the competitive inhibition is not due to steric hindrance.

“Overlapping” in the context of two epitopes shall be taken to mean that two epitopes share a sufficient number of amino acid residues to permit a compound (or protein comprising an antigen binding site thereof) that binds to one epitope to competitively inhibit the binding of a compound (or protein comprising an antigen binding site) that binds to the other epitope. For example, the “overlapping” epitopes share at least 1 or 2 or 3 or 4 or 5 or 6 or 7 or 8 or 9 or 20 amino acids.

As used herein, the term “neutralize” shall be taken to mean that a compound that binds CD131 is capable of blocking, reducing or preventing CD131-mediated signaling in a cell by IL-5 and/or GM-CSF. Methods for determining neutralization are known in the art and/or described herein.

### Treatment of Severe Asthma

The methods described herein provide methods of treating severe asthma in a subject, and/or methods of depleting and/or reducing eosinophils and/or neutrophils in a subject suffering from severe asthma, and/or methods of treating neutrophilia and/or eosinophilia in a subject suffering from severe asthma by administering a compound that binds to CD131 and/or inhibits signalling of IL-5 and GM-CSF.

As used herein, the term “severe asthma”, refers to asthma for which symptoms are only partially controlled or even uncontrolled, despite intensive treatment with standard therapies. Severe asthma can be defined according the International ERS/ATS guidelines on definition, evaluation and treatment of severe asthma (Chung et al., Eur Respir J. 2014; 43(2):343-73). According to the ERS/ATS guidelines, severe asthma is defined as asthma which would require treatment with high dose inhaled corticosteroids (ICS) plus a second controller (e.g. a Long-acting ß2 agonist, montelukast, or theophylline) and/or treatment with systemic corticosteroids to prevent it from becoming uncontrolled or which remains uncontrolled despite the treatment.

In one example, the subject is clinically diagnosed with severe asthma. Methods of clinically diagnosing severe asthma will be apparent to the skilled person and/or are described herein. Severe asthma can be defined according the International ERS/ATS guidelines on definition, evaluation and treatment of severe asthma (Chung et al., Eur Respir J. 2014; 43(2):343-73). According to the European Respiratory Society (ERS) and the American Thoracic Society (ATS) guidelines, severe asthma is defined as asthma which would require treatment with high dose inhaled corticosteroids (ICS) plus a second controller (e.g. a long-acting ß2 agonist, montelukast, or theophylline) and/or treatment with systemic corticosteroids to prevent it from becoming uncontrolled or which remains uncontrolled despite the treatment. In one example, the subject satisfies the ERS/ATS definition of severe asthma. Thus, in some examples, the subject:
(i) has, or is receiving treatment, with:
   c) high dose inhaled corticosteroid plus at least one additional controller; or
   d) oral corticosteroids for >6 months/year; and
(ii) has at least one or more, or all, of the following:
   e) asthma control test (ACT)<20 or asthma control questionnaire (ACQ)>1.5;
   f) at least 2 exacerbations in the last 12 months;
   g) at least 1 exacerbation treated in hospital or requiring mechanical ventilation in the last 12 months; and
   h) forced expiratory volume in one second (FEV₁)<80% if the FEV₁/FVC (forced vital capacity) is below the lower limit of normal.

Severe asthma can also be defined according to the Global Initiative for Asthma (GINA) definition. The GINA guidelines 2021 (Global Initiative for Asthma. Global strategy for asthma management and prevention (2021 update). Fontana, WI, USA: GINA, 2021) define severe asthma as asthma that is uncontrolled despite high dose inhaled corticosteroids (ICS)-long-acting beta2-agonist (LABA), or that requires high dose ICS-LABA to remain controlled.

Methods of diagnosing severe asthma will be apparent to the skilled person and/or are defined herein and include, for example, spirometry. In one example, the severe asthma is diagnosed using spirometery. For example, the severe asthma can be diagnosed based on a forced expiratory volume in one second (FEV1) of <80% if the FEV1/FVC (forced vital capacity) is below the lower limit of normal.

As used herein, the term “FEV₁” refers to the volume expired in the first second of maximal expiration initiated at full inspiration.

As used herein, the term “FVC” refers to the maximum volume of air that can be expired during a spirometry test.

In one example, the severe asthma is eosinophilic asthma, neutrophilic asthma, paucigranulocytic asthma or mixed granulocytic asthma.

In one example, the severe asthma is eosinophilic asthma.

In one example, the severe asthma is neutrophilic asthma.

In one example, the severe asthma is paucigranulocytic asthma.

In one example, the severe asthma is mixed granulocytic asthma.

As used herein, the term “neutrophilic asthma” refers to a subset of asthma that is characterized by an increase in the amount of neutrophils in the airways of a subject. Neutrophilic asthma can be categorized by high neutrophil counts in sputum, for example greater than 60% of sputum cells, and low eosinophilic counts in sputum, for example less than 3%. The response to treatment of neutrophilic asthma with corticosteroids is often found to be ineffective, compared to patients with eosinophilic asthma. Neutrophilic asthma is also associated with upregulated expression of IL-8, IL-17, and IFN-γ in the airways, and a low T_{H}2 inflammatory response.

As used herein, the term “eosinophilic asthma”, refers to a subset of asthma that is characterised by an increase in the levels of eosinophils in the airways of a subject. For example, eosinophilic asthma can be characterized by high eosinophil counts in sputum, e.g., greater than 3% and low neutrophil counts in sputum, e.g., less than 60%. Eosinophilic asthma is associated with an increase in IL-5 expression and a T_{H}2-dominant inflammatory response.

As used herein, the term “mixed granulocytic asthma” refers to a subset of asthma which is characterized by an increase in the amount of both neutrophils and eosinophils in the airways of a subject. For example, mixed granulocytic asthma can be characterized by high neutrophil counts in sputum, e.g., greater than 60% and high eosinophil counts in sputum, e.g., greater than 3%. Similar to neutrophilic asthma it is characterised by a low T_{H}2 inflammatory response.

As used herein, the term “paucigranulocytic asthma” refers to a subset of asthma which is characterised by no elevation in the levels of eosinophils and neutrophils in the airways of the subject. Macrophages may also play a role in development of this phenotype. For example, paucigranulocytic asthma can be categorized by low neutrophil counts in sputum, for example less than 60%, and low eosinophil counts in sputum, e.g., less than 3%.

In one example, the severe asthma is T_{H}2-high asthma or T_{H}2-low asthma. For example, the severe asthma is T_{H}2-high asthma. In another example, the severe asthma is T_{H}2-low asthma.

As used herein, the term “T_{H}2-high” asthma refers to a subset of severe asthma which is characterised by eosinophilic airway inflammation and an increased or dominant T_{H}2 inflammatory response. It will be apparent to the skilled person from the disclosure herein that the terms ‘T_{H}2-high’ asthma and ‘eosinophilic’ asthma are used interchangeably.

As used herein, the term “T_{H}2-low” asthma, refers to a subset of asthma which is characterised by with either neutrophilic, paucigranulocytic or mixed granulocytic airway inflammation and a low Th2 inflammatory response.

In one example, the compound that binds to CD131 is administered in an amount sufficient to reduce the severity of or the frequency of or prevent onset of exacerbations of severe asthma.

As used herein, the term “asthma exacerbation” (also referred to as an “asthma attack”) refers to an exaggerated lower airway response to an environmental stimulus which results in acute or subacute episode of progressively worsening shortness of breath, coughing, wheezing, or chest tightness or any combination thereof.

In one example, the subject has airway bacterial colonisation. Colonisation of potentially pathogenic bacteria in the airways is associated with an increased risk of developing severe asthma. Of significance, carriage of Streptococcus pneumoniae in the upper airways has been detected in around of 50% asthmatic children (Esposito et al., BMC Infect Dis, 2016. 16:12) and asthma is more common in children that were colonised with S. pneumoniae, Moraxella catarrhalis and/or Haemophilus influenzae during infancy. Methods for detecting and measuring bacterial colonisation of airways are known in the art. Suitable methods include culturing BAL fluid or lung tissue homogenates on horse blood agar plates to determine bacterial load, for example as described in (FitzPatrick et al., Sci Rep, 2016. 6:22751). Other methods include real time quantitative PCR on bacterial DNA isolated from lung tissue using a standard curve generated from a known quantity of bacteria.

In one example, the subject has a respiratory viral infection. In one example, the respiratory viral infection is an influenza virus infection. Respiratory viral infections may cause asthma symptoms to exacerbate and cause bacterial colonisation, if present, to disseminate into the lower airways of the subject. Accordingly, in one example, the subject has airway bacterial colonisation and a respiratory viral infection.

Compounds that Bind CD131

### Antibodies

In one example, a compound as described herein according to any example is a protein comprising an antigen binding site of an antibody. In some examples, the compound is an antibody.

Methods for generating antibodies are known in the art and/or described in Harlow and Lane (editors) Antibodies: A Laboratory Manual, Cold Spring Harbor Laboratory, (1988). Generally, in such methods CD131 or a region thereof (e.g., an extracellular domain) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

Monoclonal antibodies are one exemplary form of an antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

Alternatively, ABL-MYC technology (NeoClone, Madison WI 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

An antibody of the present disclosure may be a synthetic antibody. For example, the antibody is a chimeric antibody, a humanized antibody, a human antibody or a de-immunized antibody.

In one example, an antibody described herein is a chimeric antibody. The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass. Methods for producing chimeric antibodies are described in, e.g., U.S. Pat. Nos. 4,816,567; and 5,807,715.

The antibodies of the present disclosure may be humanized or human.

The term “humanized antibody” shall be understood to refer to a subclass of chimeric antibodies having an antigen binding site or variable region derived from an antibody from a non-human species and the remaining antibody structure based upon the structure and/or sequence of a human antibody. In a humanized antibody, the antigen-binding site generally comprises the complementarity determining regions (CDRs) from the non-human antibody grafted onto appropriate FRs in the variable regions of a human antibody and the remaining regions from a human antibody. Antigen binding sites may be wild-type (i.e., identical to those of the non-human antibody) or modified by one or more amino acid substitutions. In some instances, FR residues of the human antibody are replaced by corresponding non-human residues.

Methods for humanizing non-human antibodies or parts thereof (e.g., variable regions) are known in the art. Humanization can be performed following the method of U.S. Pat. No. 5,225,539, or U.S. Pat. No. 5,585,089. Other methods for humanizing an antibody are not excluded.

The term “human antibody” as used herein refers to antibodies having variable regions (e.g. V_{H}, V_{L}) and, optionally constant regions derived from or corresponding to sequences found in humans, e.g. in the human germline or somatic cells.

Exemplary human antibodies are described herein and include 9A2-VR24.29 (also, referred to as “CSL311”) described in WO 2017/088028 and BION-1 described in Sun et al. (1999) Blood 94:1943-1951 and/or proteins comprising variable regions thereof or derivatives thereof. These human antibodies provide an advantage of reduced immunogenicity in a human compared to non-human antibodies.

In one example, the antibody is a multispecific antibody. For instance, the compound that binds to CD131 may be a protein comprising an antigen binding site that binds to CD131 and a further antigen binding site that binds to a different antigen. Thus, in some examples, the antibody is a bispecific antibody.

### Antibody Binding Domain Containing Proteins

### Single-Domain Antibodies

In one example, a compound of the disclosure is a protein that is or comprises a single-domain antibody (which is used interchangeably with the term “domain antibody” or “dAb” or “nanobody”). A single-domain antibody, is an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from alight and one from a heavy chain). In certain examples, a single-domain antibody is a human single-domain antibody (Domantis, Inc., Waltham, MA; see, e.g., U.S. Pat. No. 6,248,516).

In one example, the single-domain antibody is a V_{H}H fragment. V_{H}H fragments consist of the variable domain (V_{H}) of camelid heavy-chain antibodies, described below.

In one example, the single-domain antibody is a V_{NAR} fragment. V_{NAR} fragments consist of the variable domain (V_{H}) of heavy-chain antibodies from cartilaginous fish, described below.

### Diabodies, Triabodies, Tetrabodies

In one example, a protein of the disclosure is or comprises a diabody, triabody, tetrabody or higher order protein complex such as those described in WO98/044001 and/or WO94/007921.

Single Chain Fv (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

### Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

### Other Antibodies and Antibody Fragments

The present disclosure also contemplates other antibodies and antibody fragments, such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab₃ (e.g., as described in EP19930302894).

### V-Like Proteins

An example of a compound of the disclosure is a T-cell receptor. T cell receptors have two V-domains that combine into a structure similar to the Fv module of an antibody. Novotny et al., Proc Natl Acad Sci USA 88: 8646-8650, 1991 describes how the two V-domains of the T-cell receptor (termed alpha and beta) can be fused and expressed as a single chain polypeptide and, further, how to alter surface residues to reduce the hydrophobicity directly analogous to an antibody scFv. Other publications describing production of single-chain T-cell receptors or multimeric T cell receptors comprising two V-alpha and V-beta domains include WO1999/045110 or WO2011/107595.

Other non-antibody proteins comprising antigen binding domains include proteins with V-like domains, which are generally monomeric. Examples of proteins comprising such V-like domains include CTLA-4, CD28 and ICOS. Further disclosure of proteins comprising such V-like domains is included in WO1999/045110.

### Adnectins

In one example, a compound of the disclosure is an adnectin. Adnectins are based on the tenth fibronectin type III (¹⁰Fn3) domain of human fibronectin in which the loop regions are altered to confer antigen binding. For example, three loops at one end of the β-sandwich of the ¹⁰Fn3 domain can be engineered to enable an Adnectin to specifically recognize an antigen. For further details see US20080139791 or WO2005/056764.

### Anticalins

In a further example, a compound of the disclosure is an anticalin. Anticalins are derived from lipocalins, which are a family of extracellular proteins which transport small hydrophobic molecules such as steroids, bilins, retinoids and lipids. Lipocalins have a rigid β-sheet secondary structure with a plurality of loops at the open end of the conical structure which can be engineered to bind to an antigen. Such engineered lipocalins are known as anticalins. For further description of anticalins see U.S. Pat. No. 7,250,297B1 or US20070224633.

### Affibodies

In a further example, a compound of the disclosure is an affibody. An affibody is a scaffold derived from the Z domain (antigen binding domain) of Protein A of Staphylococcus aureus which can be engineered to bind to antigen. The Z domain consists of a three-helical bundle of approximately 58 amino acids. Libraries have been generated by randomization of surface residues. For further details see EP1641818.

### Avimers

In a further example, a compound of the disclosure is an Avimer. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see WO2002/088171.

### DARPins

In a further example, a compound of the disclosure is a Designed Ankyrin Repeat Protein (DARPin). DARPins are derived from Ankyrin which is a family of proteins that mediate attachment of integral membrane proteins to the cytoskeleton. A single ankyrin repeat is a 33 residue motif consisting of two a-helices and a p-turn. They can be engineered to bind different target antigens by randomizing residues in the first a-helix and a β-turn of each repeat. Their binding interface can be increased by increasing the number of modules (a method of affinity maturation). For further details see US20040132028.

### De-Immunized Proteins

The present disclosure also contemplates a de-immunized antibody or protein.

De-immunized antibodies and proteins have one or more epitopes, e.g., B cell epitopes or T cell epitopes removed (i.e., mutated) to thereby reduce the likelihood that a mammal will raise an immune response against the antibody or protein. Methods for producing de-immunized antibodies and proteins are known in the art and described, for example, in WO2000/34317, WO2004/108158 and WO2004/064724.

Methods for introducing suitable mutations and expressing and assaying the resulting protein will be apparent to the skilled artisan based on the description herein.

### Mutations to Proteins

The present disclosure also contemplates mutant forms of a protein of the disclosure. For example, such a mutant protein comprises one or more conservative amino acid substitutions compared to a sequence set forth herein. In some examples, the protein comprises 30 or fewer or 20 or fewer or 10 or fewer, e.g., 9 or 8 or 7 or 6 or 5 or 4 or 3 or 2 conservative amino acid substitutions. A “conservative amino acid substitution” is one in which the amino acid residue is replaced with an amino acid residue having a similar side chain and/or hydropathicity and/or hydrophilicity.

In one example, a mutant protein has only, or not more than, one or two or three or four or five or six conservative amino acid changes when compared to a naturally occurring protein. Details of conservative amino acid changes are provided below. As the skilled person would be aware, e.g., from the disclosure herein, such minor changes can reasonably be predicted not to alter the activity of the protein.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), P-branched side chains (e.g., threonine, valine, isoleucine) and aromatic side chains (e.g., tyrosine, phenylalanine, tryptophan, histidine).

The present disclosure also contemplates non-conservative amino acid changes (e.g., substitutions) in a protein of the present disclosure, e.g., in a CDR, such as CDR3. In one example, the protein comprises fewer than 6 or 5 or 4 or 3 or 2 or 1 non-conservative amino acid substitutions, e.g., in a CDR3, such as in a CDR3.

The present disclosure also contemplates one or more insertions or deletions compared to a sequence set forth herein. In some examples, the protein comprises 10 or fewer, e.g., 9 or 8 or 7 or 6 or 5 or 4 or 3 or 2 insertions and/or deletions.

### Constant Regions

The present disclosure encompasses proteins and/or antibodies described herein comprising a constant region of an antibody. This includes antigen binding fragments of an antibody fused to a Fc.

Sequences of constant regions useful for producing the proteins of the present disclosure may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol; 75: 12161-12168, 2001).

In another example, the Fc region is a chimeric Fc region, e.g., comprising at least one C_{H}2 domain from an IgG₄ antibody and at least one C_{H}3 domain from an IgG₁ antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Additional Modifications

The present disclosure also contemplates additional modifications to an antibody or protein of the disclosure.

For example, the antibody comprises one or more amino acid substitutions that increase the half-life of the protein. For example, the antibody comprises a Fc region comprising one or more amino acid substitutions that increase the affinity of the Fc region for the neonatal Fc receptor (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half-life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

The protein may be a fusion protein. Thus, in one example, the protein additionally comprises albumin, a functional fragment or variant thereof. In one example, the albumin, functional fragment or variant thereof is serum albumin, such as human serum albumin. In one example, the albumin, functional fragment or variant thereof, comprises one or more amino acid substitutions, deletions or insertions, e.g., no more than 5 or 4 or 3 or 2 or 1 substitutions. Amino acid substitutions suitable for use in the present disclosure will be apparent to the skilled person and include naturally-occurring substitutions and engineered substitutions such as those described, for example, in WO2011/051489, WO2014/072481, WO2011/103076, WO2012/112188, WO2013/075066, WO2015/063611 and WO2014/179657.

In one example, the protein of the disclosure additionally comprises a soluble complement receptor or functional fragment or variant thereof. In one example, the protein additionally comprises a complement inhibitor.

### Exemplary CD131-Binding Proteins

Exemplary variable regions containing CD131-binding proteins have been previously described in WO 2017/088028 and/or are described in Table 1.

TABLE 1
Sequences of exemplary CD131-binding proteins

| | | V_{L} amino acid SEQ | V_{H} amino acid SEQ |
|---|---|---|---|
| | Antibody name | ID NO. | ID NO. |
| 1 | 9A2 | 5 | 20 |
| 2 | 9A2-VR1 | 6 | 20 |
| 3 | 9A2-VR2 | 7 | 20 |
| 4 | 9A2-VR3 | 8 | 20 |
| 5 | 9A2-VR4 | 9 | 20 |
| 6 | 9A2-VR5 | 10 | 20 |
| 7 | 9A2-VR6 | 11 | 20 |
| 8 | 9A2-VR8 | 12 | 20 |
| 9 | 9A2-VR9 | 13 | 20 |
| 10 | 9A2-VR11 | 14 | 20 |
| 11 | 9A2-VR12 | 15 | 20 |
| 12 | 9A2-VR13 | 16 | 20 |
| 13 | 9A2-VR14 | 17 | 20 |
| 14 | 9A2-VR16 | 18 | 20 |
| 15 | 9A2-VR19 | 19 | 20 |
| 16 | 9A2-VR20 | 5 | 21 |
| 17 | 9A2-VR21 | 5 | 22 |
| 18 | 9A2-VR22 | 5 | 23 |
| 19 | 9A2-VR23 | 5 | 24 |
| 20 | 9A2-VR24 | 5 | 25 |
| 21 | 9A2-VR26 | 5 | 26 |
| 22 | 9A2-VR27 | 5 | 27 |
| 23 | 9A2-VR28 | 5 | 28 |
| 24 | 9A2-VR31 | 5 | 29 |
| 25 | 9A2-VR32 | 5 | 30 |
| 26 | 9A2-VR33 | 5 | 31 |
| 27 | 9A2-VR34 | 5 | 32 |
| 28 | 9A2-VR35 | 5 | 33 |
| 29 | 9A2-VR36 | 5 | 34 |
| 30 | 9A2-VR37 | 5 | 35 |
| 31 | 9A2-VR38 | 5 | 36 |
| 32 | 9A2-VR39 | 5 | 37 |
| 33 | 9A2-VR40 | 5 | 38 |
| 34 | 9A2-VR41 | 5 | 39 |
| 35 | 9A2-VR42 | 5 | 40 |
| 36 | 9A2-VR43 | 5 | 41 |
| 37 | 9A2-VR44 | 5 | 42 |
| 38 | 9A2-VR45 | 5 | 43 |
| 39 | 9A2-VR46 | 5 | 44 |
| 40 | 9A2-VR47 | 5 | 45 |
| 41 | 9A2-VR48 | 5 | 46 |
| 42 | 9A2-VR49 | 5 | 47 |
| 43 | 9A2-VR50 | 5 | 48 |
| 44 | 9A2-VR24.04 | 5 | 49 |
| 45 | 9A2-VR24.07 | 5 | 50 |
| 46 | 9A2-VR24.10 | 5 | 51 |
| 47 | 9A2-VR24.12 | 5 | 52 |
| 48 | 9A2-VR24.19 | 5 | 53 |
| 49 | 9A2-VR24.24 | 5 | 54 |
| 50 | 9A2-VR24.76 | 5 | 55 |
| 51 | 9A2-VR24.78 | 5 | 56 |
| 52 | 9A2-VR24.81 | 5 | 57 |
| 53 | 9A2-VR24.82 | 5 | 58 |
| 54 | 9A2-VR24.84 | 5 | 59 |
| 55 | 9A2-VR24.87 | 5 | 60 |
| 56 | 9A2-VR24.91 | 5 | 61 |
| 57 | 9A2-VR24.93 | 5 | 62 |
| 58 | 9A2-VR24.27 | 5 | 63 |
| 59 | 9A2-VR24.29 | 5 | 64 |
| 60 | 9A2-VR24.30 | 5 | 65 |
| 61 | 9A2-VR24.33 | 5 | 66 |
| 62 | 9A2-VR24.44 | 5 | 67 |
| 63 | 9A2-VR24.97 | 5 | 68 |
| 64 | 9A2-VR24.98 | 5 | 69 |
| 65 | 9A2-VR24.102 | 5 | 70 |
| 66 | 9A2-VR24.107 | 5 | 71 |
| 67 | 9A2-VR24.110 | 5 | 72 |
| 68 | 9A2-VR24.111 | 5 | 73 |
| 69 | 9A2-VR24.55 | 5 | 74 |
| 70 | 9A2-VR24.56 | 5 | 75 |
| 71 | 9A2-VR24.57 | 5 | 76 |
| 72 | 9A2-VR24.122 | 5 | 77 |
| 73 | 9A2-VR24.124 | 5 | 78 |
| 74 | 9A2-VR24.131 | 5 | 79 |
| 75 | 9A2-VR39.01 | 5 | 80 |
| 76 | 9A2-VR39.02 | 5 | 81 |
| 77 | 9A2-VR39.04 | 5 | 82 |
| 78 | 9A2-VR39.05 | 5 | 83 |
| 79 | 9A2-VR39.06 | 5 | 84 |
| 80 | 9A2-VR39.11 | 5 | 85 |
| 81 | 9A2-VR39.12 | 5 | 86 |
| 82 | 9A2-VR39.16 | 5 | 87 |
| 83 | 9A2-VR39.17 | 5 | 88 |
| 84 | 9A2-VR39.18 | 5 | 89 |
| 85 | 9A2-VR39.19 | 5 | 90 |
| 86 | 9A2-VR39.21 | 5 | 91 |
| 87 | 9A2-VR39.22 | 5 | 92 |
| 88 | 9A2-VR39.23 | 5 | 93 |
| 89 | 9A2-VR39.24 | 5 | 94 |
| 90 | 9A2-VR39.97 | 5 | 95 |
| 91 | 9A2-VR39.98 | 5 | 96 |
| 92 | 9A2-VR39.102 | 5 | 97 |
| 93 | 9A2-VR39.103 | 5 | 98 |
| 94 | 9A2-VR39.105 | 5 | 99 |
| 95 | 9A2-VR39.109 | 5 | 100 |
| 96 | 9A2-VR39.110 | 5 | 101 |
| 97 | 9A2-VR39.111 | 5 | 102 |
| 98 | 9A2-VR39.112 | 5 | 103 |
| 99 | 9A2-VR39.116 | 5 | 104 |
| 100 | 9A2-VR39.27 | 5 | 105 |
| 101 | 9A2-VR39.28 | 5 | 106 |
| 102 | 9A2-VR39.46 | 5 | 107 |
| 103 | 9A2-VR39.122 | 5 | 108 |
| 104 | 9A2-VR39.139 | 5 | 109 |
| 105 | 9A2-VR39.140 | 5 | 110 |
| 106 | 9A2-VR39.148 | 5 | 111 |
| 107 | 9A2-VR39.162 | 5 | 112 |
| 108 | 9A2-VR39.77 | 5 | 113 |
| 109 | 9A2-VR39.93 | 5 | 114 |
| 110 | 9A2-VR39.174 | 5 | 115 |
| 111 | 9A2-VR39.177 | 5 | 116 |

### Protein Production

In one example, a protein described herein according to any example is produced by culturing a hybridoma under conditions sufficient to produce the protein, e.g., as described herein and/or as is known in the art.

### Recombinant Expression

In another example, a protein described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Methods for isolating a protein are known in the art and/or described herein.

Where a protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO1999/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

Nucleic Acid Compounds that Bind to CD131

In one example, the compound that binds to CD131 is a nucleic acid aptamer (adaptable oligomer). Aptamers are single stranded oligonucleotides or oligonucleotide analogs that are capable of forming a secondary and/or tertiary structure that provides the ability to bind to a particular target molecule, such as a protein or a small molecule, e.g., CD131. Thus, aptamers are the oligonucleotide analogy to antibodies. In general, aptamers comprise about 15 to about 100 nucleotides, such as about 15 to about 40 nucleotides, for example about 20 to about 40 nucleotides, since oligonucleotides of a length that falls within these ranges can be prepared by conventional techniques.

An aptamer can be isolated from or identified from a library of aptamers. An aptamer library is produced, for example, by cloning random oligonucleotides into a vector (or an expression vector in the case of an RNA aptamer), wherein the random sequence is flanked by known sequences that provide the site of binding for PCR primers. An aptamer that provides the desired biological activity (e.g., binds specifically to CD131) is selected. An aptamer with increased activity is selected, for example, using SELEX (Sytematic Evolution of Ligands by EXponential enrichment). Suitable methods for producing and/or screening an aptamer library are described, for example, in Elloington and Szostak, Nature 346:818-22, 1990; U.S. Pat. No. 5,270,163; and/or U.S. Pat. No. 5,475,096.

### Assaying Activity of a Compound

### Binding to CD131

Methods for assessing binding of a candidate compound to a protein (e.g., CD131) are known in the art, e.g., as described in Scopes (In: Protein purification: principles and practice, Third Edition, Springer Verlag, 1994). Such a method generally involves labeling the protein and contacting it with immobilized compound. Following washing to remove non-specific bound protein, the amount of label and, as a consequence, bound protein is detected. Of course, the protein can be immobilized and the compound labeled. Panning-type assays can also be used. Alternatively, or additionally, surface plasmon resonance assays can be used. The level of binding can also be conveniently determined using a biosensor.

Optionally, the dissociation constant (Kd) of a compound for CD131 or an epitope thereof is determined. The “Kd” or “Kd value” for a compound that binds to CD131 is in one example measured by a radiolabeled or fluorescently-labeled CD131 binding assay. This assay equilibrates the compound with a minimal concentration of labeled CD131 in the presence of a titration series of unlabeled CD131. Following washing to remove unbound CD131, the amount of label is determined, which is indicative of the Kd of the protein.

According to another example the Kd or Kd value is measured by using surface plasmon resonance assays, e.g., using BIAcore surface plasmon resonance (BIAcore, Inc., Piscataway, NJ) with immobilized CD131 or a region thereof.

### Epitope Mapping

In another example, the epitope bound by a protein described herein is mapped. Epitope mapping methods will be apparent to the skilled artisan. For example, a series of overlapping peptides spanning the CD131 sequence or a region thereof comprising an epitope of interest, e.g., peptides comprising 10-15 amino acids are produced. The protein is then contacted to each peptide and the peptide(s) to which it binds determined. This permits determination of peptide(s) comprising the epitope to which the protein binds. If multiple non-contiguous peptides are bound by the protein, the protein may bind a conformational epitope.

Alternatively, or in addition, amino acid residues within CD131 are mutated, e.g., by alanine scanning mutagenesis, and mutations that reduce or prevent protein binding are determined. Any mutation that reduces or prevents binding of the protein is likely to be within the epitope bound by the protein.

A further method is exemplified herein, and involves binding CD131 or a region thereof to an immobilized protein of the present disclosure and digesting the resulting complex with proteases. Peptide that remains bound to the immobilized protein are then isolated and analyzed, e.g., using mass spectrometry, to determine their sequence.

A further method involves converting hydrogens in CD131 or a region thereof to deutrons and binding the resulting protein to an immobilized protein of the present disclosure. The deutrons are then converted back to hydrogen, the CD131 or region thereof isolated, digested with enzymes and analyzed, e.g., using mass spectrometry to identify those regions comprising deutrons, which would have been protected from conversion to hydrogen by the binding of a protein described herein.

Alternatively, the epitope to which the protein binds can be determined by X-ray crystallography. For example, a complex between the protein and CD131 is formed and then crystalized. The resulting crystals are then subjected to x-ray diffraction analysis to determine the atomic co-ordinates of the amino acids in the complex. The epitope comprises the amino acids in CD131 that are in contact with the protein, according to the atomic co-ordinates determined from the x-ray diffraction.

### Determining Competitive Binding

Assays for determining a protein that competitively inhibits binding of antibody described herein will be apparent to the skilled artisan. For example, exemplary antibody CSL311 is conjugated to a detectable label, e.g., a fluorescent label or a radioactive label. The labeled antibody and the test protein are then mixed and contacted with CD131 or a region thereof (e.g., a polypeptide comprising SEQ ID NO: 1) or a cell expressing same. The level of labeled CSL311 is then determined and compared to the level determined when the labeled antibody is contacted with the CD131, region or cells in the absence of the protein. If the level of labeled CSL311 is reduced in the presence of the test protein compared to the absence of the protein, the protein is considered to competitively inhibit binding of CSL311 to CD131.

Optionally, the test protein is conjugated to different label to CSL311. This alternate labeling permits detection of the level of binding of the test protein to CD131 or the region thereof or the cell.

In another example, the protein is permitted to bind to CD131 or a region thereof (e.g., a polypeptide comprising SEQ ID NO: 1) or a cell expressing same prior to contacting the CD131, region or cell with CSL311. A reduction in the amount of bound CSL311 in the presence of the protein compared to in the absence of the protein indicates that the protein competitively inhibits CSL311 binding to CD131. A reciprocal assay can also be performed using labeled protein and first allowing CSL311 to bind to CD131. In this case, a reduced amount of labeled protein bound to CD131 in the presence CSL311 compared to in the absence of CSL311 indicates that the protein competitively inhibits binding of CSL311 to CD131.

Any of the foregoing assays can be performed with a mutant form of CD131 and/or SEQ ID NO: 1 and/or a ligand binding region of CD131 to which CSL311 binds, e.g., as described herein.

### Determining Neutralization

In one example, the compound that binds to CD131 reduces or prevents binding of IL-3, IL-5 and/or GM-CSF to a receptor comprising CD131 (e.g., an IL-3R, an IL-5R and/or a GM-CSF-R, respectively). These assays can be performed as a competitive binding assay using labeled IL-3/IL-5/GM-CSF and/or labeled compound. For example, cells expressing the relevant receptor is contacted with IL-3/IL-5/GM-CSF in the presence or absence of a CD131-binding compound and the amount of bound label detected. A reduction in the amount of bound label in the presence of the CD131-binding compound compared to in the absence of the compound indicates that the compound reduces or prevents binding of IL-3/IL-5/GM-CSF to a receptor comprising CD131. By testing multiple concentrations of the compound an IC₅₀ is determined, i.e., a concentration of the compound that reduces the amount of IL-3/IL-5/GM-CSF that binds to a receptor comprising CD131, or an EC₅₀ can be determined, i.e., a concentration of the protein that achieves 50% of the maximum inhibition of binding of IL-3/IL-5/GM-CSF to CD131 achieved by the compound.

In a further example, the CD131-binding compound reduces or prevents IL-3/IL-5/GM-CSF-mediated proliferation of leukemic cell line TF-1. For example, TF-1 cells are cultured without IL-3/IL-5/GM-CSF for a time sufficient for them to stop proliferating (e.g., 24-48 hours). Cells are then cultured in the presence of IL-3/IL-5/GM-CSF and various concentrations of the CD131-binding compound. Control cells are not contacted with the compound (positive control) or IL-3/IL-5/GM-CSF (negative control). Cell proliferation is then assessed using a standard technique, e.g., ³H-thymidine incorporation. A CD131-binding compound that reduces or prevents cell proliferation in the presence of IL-3 to a level less than the positive control is considered to neutralize IL-3 signaling. By testing multiple concentrations of the CD131-binding compound, an IC₅₀ is determined.

In a further example, a CD131-binding compound of the disclosure affects an immune cell. For example, the CD131-binding compound reduces or inhibits activation of isolated human neutrophils by GM-CSF as determined by reducing or inhibiting GM-CSF-induced increase in neutrophil cell size. For example, neutrophils (e.g., about 1×10⁵ cells) are cultured in the presence of the CD-131-binding protein and GM-CSF for a suitable time (e.g., about 24 hours). Cells are then fixed (e.g., with formaldehyde) and analyzed for forward scatter using flow cytometry.

In a further example, the CD131-binding compound reduces or prevents activation of human peripheral blood eosinophils by IL-5 as determined by assessing change in forward scatter assessed by flow cytometry. For example, eosinophils (e.g., about 1×10⁵ cells) are cultured in the presence of a CD131-binding compound and IL-5 for a suitable time (e.g., about 24 hours). Cells are then fixed (e.g., in formaldehyde) and assessed for change in forward scatter, e.g., using flow cytometry.

In a further example, a CD131-binding compound of the disclosure reduces or prevents survival of human peripheral blood eosinophils. For example, the compound prevents reduces or prevents survival of eosinophils in the presence of IL-5 and/or GM-CSF and/or IL-3. For example, eosinophils (e.g., about 1×10⁴ cells) are cultured in the presence of a CD131-binding compound and IL-5 and/or GM-CSF and/or IL-3 for a suitable time (e.g., about 5 days) and cell numbers assessed using a standard method (e.g., a ViaLight Plus Kit from Lonza).

In a further example, a CD131-binding compound reduces accumulation of or survival of or induces death of immune cells (e.g., eosinophils) from bronchoalveolar (BAL) fluid or lung tissue from a subject suffering from severe asthma. For example, the immune cells are cultured in the presence of IL-3 and/or IL-5 and/or GM-CSF and the protein or antibody. Cell death is then assessed using standard methods, e.g., by detecting Annexin-V expression, e.g., using fluorescence activated cell sorting.

Other methods for assessing neutralization of GM-CSF, IL-5 or IL-3 signaling are contemplated by the present disclosure.

### Therapeutic Efficacy

The therapeutic efficacy of a compound that binds to CD131 can be assessed by comparing the degree of severity of the disease or symptoms in subjects administered with the compound relative to subjects not administered the compound. Alternatively, or additionally, therapeutic efficacy of candidate compounds can be assessed in an animal model. Suitable assays for assessing therapeutic efficacy are described.

In one example, the efficacy of a protein to treat a condition is assessed using an in vivo assay.

In one example, the CD131-binding compound is administered to a non-human animal (e.g., a non-human primate) and the number/level of immune cells, e.g., eosinophils or neutrophils, in circulation or in a tissue or other sample (e.g., skin tissue at the site of inflammation) is assessed. A CD131-binding compound that reduces the number/level of immune cells compared to prior to administration and/or in a control mammal to which the protein has not been administered is considered suitable for treating the disease or condition.

In one example, a CD131-binding compound is tested in a model of severe asthma. Exemplary models of severe asthma are described in Ouyang S et al., JCI Insight 2020. In such models, a non-human mammal (e.g., a rodent, such as a mouse) is sensitised, and subsequently challenged, with a house dust mite extract (HDM). In another model, a non-human mammal (e.g., a rodent, such as a mouse) is sensitised, and subsequently challenged, with an Aspergillus extract (ASP). Following exposure, the mammal is administered a CD131-binding protein and the level of lung inflammation and/or the number of eosinophils and/or neutrophils in the lung is assessed or estimated using standard techniques. A CD131-binding protein that reduces lung inflammation and/or the number of eosinophils and/or neutrophils is considered useful for treating severe asthma.

In one example, a CD131-binging protein is tested in a model of eosinophil-associated COPD/asthma overlap (ACO). In such models, a non-human mammal (e.g., a rodent, such as a mouse) is sensitised, and subsequently challenged, with a house dust mite extract (HDM) and porcine pancreatic elastase (PPE). Following exposure, the mammal is administered a CD131-binding protein and the level of lung inflammation and/or the number of eosinophils and/or neutrophils in the lung is assessed or estimated using standard techniques. A CD131-binding protein that reduces lung inflammation and/or the number of eosinophils and/or neutrophils is considered useful for treating eosinophil-associated COPD/asthma overlap (ACO).

In another example, the CD131-binding protein is administered to a non-human animal (e.g., a non-human primate) and the number/level of immune cells, e.g., eosinophils, in circulation or in a tissue or other sample (e.g., BAL fluid) is assessed. A CD131-binding protein that reduces the number/level of immune cells compared to prior to administration and/or in a control mammal to which the protein has not been administered is considered suitable for treating the disease or condition.

In some examples, assessing the therapeutic efficacy of a compound comprises detecting and/or quantifying the level of expression of a biomarker in the subject. Suitable biomarkers for assessing efficacy include pro-type I collagen (Col1a1), beta chain cytokine receptor (CSF2RB), Interleukin 13 (IL-13), IL-4, IL-17 and C—C Motif Chemokine Ligand 17 (CCL17).

Detecting and/or quantifying biomarkers can be performed by any method known in the art. For instance, in one example, the levels of biomarkers are assessed using mass spectrometry. The mass spectrometry may be performed in conjunction with ultra-performance liquid chromatography (UPLC), high-performance liquid chromatography (HPLC), gas chromatography (GC), gas chromatography/mass spectroscopy (GC/MS), and UPLC, for example. Other methods of assessing levels of biomarkers include biological methods, such as but not limited to ELISA assays, Western Blot and multiplexed immunoassays etc. Other techniques may include using quantitative arrays, PCR, Northern Blot analysis. To determine levels of components or factors, it is not necessary that an entire component, e.g., a full length protein or an entire RNA transcript, be present or fully sequenced. In other words, determining levels of, for example, a fragment of protein being analyzed may be sufficient to conclude or assess that the level of the biomarker being analyzed is increased or decreased. Similarly, if, for example, arrays or blots are used to determine component levels, the presence/absence/strength of a detectable signal may be sufficient to assess levels of biomarkers.

To assess levels of biomarkers, a sample may be taken from the subject. The sample may or may not processed prior assaying levels of the components of the biomarker profile. For example, whole blood may be taken from an individual and the blood sample may be processed, e.g., centrifuged, to isolate plasma or serum from the blood. The sample may or may not be stored, e.g., frozen, prior to processing or analysis.

Biological samples that may be tested in a method of the invention include whole blood, blood serum, plasma, tracheal aspirate, BALF, urine, saliva, or other bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner.

### Compositions

In one example, a CD131-binding protein as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, intrapolyp and intracranial injection or infusion techniques.

In one example, the CD131-binding protein of the disclosure is formulated for administration intraveneously. For example, the CD131-binding protein of the disclosure is administered intraveneously.

In one example, the CD131-binding protein of the disclosure is formulated for administration subcutaneously. For example, the CD131-binding protein of the disclosure is administered subcutaneously.

Methods for preparing a CD131-binding protein into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this disclosure are particularly useful for parenteral administration, such as intravenous administration or administration into a body cavity or lumen of an organ or joint. The compositions for administration will commonly comprise a solution of a CD131-binding compound dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of a CD131-binding compound of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, a CD131-binding compound of the present disclosure will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver a CD131-binding compound of the present disclosure.

WO2002/080967 describes compositions and methods for administering aerosolized compositions comprising antibodies for the treatment of respiratory conditions, which are also suitable for administration of compounds in accordance with the methods of the present disclosure.

### Combination Therapies

In one example, a CD131-binding compound of the present disclosure is administered in combination with another compound or therapy useful for treating a condition described herein, either as combined or additional treatment steps or as additional components of a therapeutic formulation. For example, the other compound or therapy is a standard of care therapy.

In one example, the additional therapy (or standard of care therapy) comprises an anti-inflammatory compound, and immuno-modulator, an immunosuppressant, a corticosteroid, a β2 agonist, leukotriene receptor antagonist, muscarinic antagonist, theophylline, magnesium sulfate, anti-thymic stromal lymphopoietin (TSLP) antibody, tiotropium, anti-IL-5 antibody, anti-IL-13 antibody and/or anti-1L-17A antibody. In one example, the additional therapy is an anti-inflammatory compound.

In one example, the additional therapy is an immunomodulator or an immunosuppressant.

In one example, the additional therapy is a corticosteroid, e.g. a glucocorticoid. For example, the corticosteroid is an inhaled corticosteroid or an oral corticosteroid. For example, the corticosteroid is glucocorticoid, beclometasone (e.g., Qvar®, Beconase AQ®, budesonide (e.g., Pulmicort Flexhaler®), ciclesonide (e.g., Alvesco®), or fluticasone (e.g., Flovent® HFA).

In one example, the additional therapy is a β2 agonist. For example, the β2 agonist is salbutamol (e.g., Ventolin®), terbutaline sulfate (e.g., Bricanyl®, Marex®), formoterol (e.g., Perforomist®), or salmeterol (e.g., Serevent®).

In one example, the additional therapy is a leukotriene receptor antagonist. For example, the leukotriene receptor antagonist is montelukast (e.g., Singulair®).

In one example, the additional therapy is a muscarinic antagonist. For example, the muscarinic antagonist is ipratropium bromide (e.g., Atrovent®).

In one example, the additional therapy is a theophylline. For example, the theophylline is aminophylline (e.g., Euphyllin®).

In one example, the additional therapy is magnesium sulfate.

In one example, the additional therapy is a mast cell stabilizer. For example, the mast cell stabilizer is sodium cromoglycate or nedocromil (e.g., Tilade®).

In one example, the additional therapy is an anti-IL-5 antibody, e.g., mepolizumab (e.g., Nucala®) or reslizumab (e.g., Cinqair®) or benralizumab (e.g., Fasenra®).

In one example, the additional therapy is an anti-1L-13 antibody, e.g., dupilumab (Dupixent®).

In one example, the additional therapy is an anti-1L-17A antibody, e.g., secukinumab (e.g., Cosentyx®).

In one example, the additional therapy is an anti-thymic stromal lymphopoietin (TSLP) antibody, e.g., tezepelumab (e.g., Tezspire®).

In one example, the additional therapy is tiotropium (e.g., Spiriva®).

One skilled in the art would be able, by routine experimentation, to determine what an effective, non-toxic amount of a CD131-binding protein composition would be for the purpose of treating severe asthma as described herein. For example, a therapeutically active amount of a polypeptide may vary according to factors such as the disease stage, age, sex and weight of the subject, and the ability of the binding protein to elicit a desired response in the subject. The dosage regimen may be adjusted to provide the optimum therapeutic response. For example, several divided doses may be administered daily, or the dose may be proportionally reduced as indicated by the exigencies of the therapeutic situation. Generally, however, an effective dosage is expected to be in the range of about 1 to 200 mg/kg body weight.

### Kits

Another example of the disclosure provides kits containing compounds useful for the treatment of severe asthma as described above.

In one example, the kit comprises (a) a container comprising a compound that binds to CD131 as described herein, optionally in a pharmaceutically acceptable carrier or diluent; and (b) a package insert with instructions for treating severe asthma in a subject.

In one example, the kit comprises (a) a container comprising a compound that binds to CD131 as described herein, optionally in a pharmaceutically acceptable carrier or diluent; and (b) a package insert with instructions for depleting and/or reducing eosinophils and/or neutrophils in a subject suffering from severe asthma.

In one example, the kit comprises (a) a container comprising a compound that binds to CD131 as described herein, optionally in a pharmaceutically acceptable carrier or diluent; and (b) a package insert with instructions for treating neutrophilia and/or eosinophilia in a subject suffering from severe asthma.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for treating severe asthma as described herein and may have a sterile access port (for example, the container may be an intravenous solution bag or a vial having a stopper pierceable by a hypodermic injection needle). At least one active agent in the composition is the compound that binds to CD131. The label or package insert indicates that the composition is administered to a subject eligible for treatment, e.g., one having severe asthma as described herein, with specific guidance regarding dosing amounts and intervals of compound and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The present disclosure includes the following non-limiting Examples.

### EXAMPLES

### Example 1: Materials and Methods

### Asthma Cohort Analysis

To evaluate βc receptor expression in patients with asthma, CSF2RB gene was measured in the bronchial biopsies obtained from a cohort of asthmatics (n=26), consisting of 12 patients with mild-to-moderate disease and 14 patients with severe disease based on GINA definitions. Fibrosis-associated gene COL1A1 was also measured.

### Animals

All animal experiments were approved at RMIT University in accordance with the guidelines of ARRIVE and National Health and Medical Research Council of Australia (NHMRC). Transgenic (Tg) mice devoid of murine βc/βIL-3 receptors and expressing the human (h)pc receptor (hβcTg mice) were used in this study, which respond to mouse GM-CSF and IL-5 but not IL-3.

To model steroid-refractory severe asthma, 8-12 weeks female hβcTg mice were sensitised (s.c. injection) with house dust mite extract (HDM, Dermatophagoides pteronyssinus, Greer Laboratories, US; 100 μg) that was emulsified in complete Freund's adjuvant (CFA, Sigma-Aldrich, US). Eight days later, CFA-HDM treated mice were further challenged with HDM via the intranasal route (25 μg in 35 L saline, daily) for 4 consecutive days, whereas control mice were instilled with saline (SAL). Endpoint analysis were performed 24 hours post the last HDM treatment. In a separate cohort of mice, a human monoclonal antibody against βc (CSL311, 9A2-VR24 clone) or matching isotype control (BM4 clone, both from CSL Ltd, Australia) was therapeutically administered on day 8 and day 10 prior to the HDM challenge. Antibodies were delivered via i.v. injections at 50 mg/kg. For chronic Aspergillus (ASP) exposure mice were treated with 50 μg of extract from Aspergillus Fumigatus that was purchased from Stallergenes Greer (item XPM3D3A25, lot 365084) once a week for 4 weeks followed by 20 μg ASP every second day for 4 weeks. Isotype and CSL311 groups receive intravenous injection of antibody via the tail vein for the final 4 weeks. Mice were analysed 48 hours after the last ASP challenge.

### Lung Function

Respiratory mechanics were measured in vivo using a flexiVent system (flexiVent FX1, SCIREQ Inc, Canada). Briefly, mice were anaesthetised with ketamine (125 mg/kg) and xylazine (25 mg/kg) before being tracheostomized and cannulated. Mice were then connected to the flexiVent where baseline parameters were first obtained. This was followed by the stepwise delivery of nebulized saline and increasing doses of methacholine (MCh, 3-50 mg/mL) to the lungs to assess respiratory resistance, compliance and elastance.

### Bronchoalveolar Lavage and Tissue Collection

After lung function testing, mice were euthanised by pentobarbital overdosing. This was followed by bronchoalveolar lavage (BAL) to collect BAL-fluid and BAL cells, and total cells recovered was enumerated using a hemocytometer. Cytospins were prepared and stained with a Hemacolor® Rapid staining kit (Merck Millipore, Germany) for differential cell analysis. BAL-fluid was then centrifuged to collect the cell-free supernatant (BALF) for biochemical assays. Blood was drawn by cardiac puncture for haematologic analysis (Cell-Dyn Emerald, Abbott Laboratories, US). Lungs were perfused free of blood with ice-old PBS. The superior lobe of the lungs was excised for flow cytometry analysis and the left lobe was fixed in 10% neutral-buffered formalin for histological analysis. The remaining lung lobes were snap-frozen in liquid nitrogen prior to −80° C. storage.

### Flow Cytometry

The superior lung lobe was finely minced and digested in Liberase™ TM (Sigma-Aldrich, US) at 37° C. with constant shaking. Single cell suspensions were prepared by passing the digested tissue through a 21G needle and then a 40 μm cell strainer. This cell suspension was centrifuged and treated with ACK red cell lysis buffer. After blocking with CD16/CD32 antibody, cells were then stained with a myeloid cell antibody cocktail consisting of FITC-CD45, PE-Siglec F, APC-F4/80, eFluor 450-CD11b, PE/Cy7-CD11c, PerCp/eFluor710-Ly6G and LIVE/DEAD™ Fixable Yellow Dead Cell Stain. Following staining, cells were fixed with an IC Fixation Buffer before analysed on a BD FACSAria flow cytometer (BD, US). All antibodies and reagents were from Thermofisher Scientific, US unless otherwise stated. Data were analysed with FlowJo v10.7.2 software (BD, US).

### Histology and Immunofluorescence Staining

The left lung lobe was fixed in 10% neutral-buffered formalin before processed, paraffin-embedded and sectioned at 4 μm. Sections were stained with hematoxylin and eosin (H&E) for assessment of lung injury, and Masson's trichrome (MT) for quantification of airway collagen. Stained whole lung sections were scanned using a slide scanner (VS-120, Olympus, Japan) and morphometric analysis was performed using CellSens Dimensions software (Olympus, Japan). Lung inflammation was scored in a blinded manner on a scale of 0 to 3 (none to severe) in peribronchial, perivascular and interstitial/alveolar regions individually based on the degree of inflammatory cell infiltration. Morphometric evaluation of airway collagen was analysed on a minimum of four bronchioles per section (100 to 350 μm diameter). A 30 μm band (region of interest, ROI) was circled around the subepithelial layer of the selected bronchioles. MT-positive area was quantified within the ROI and expressed as ROI fractions.

### Quantitative Reverse Transcription PCR (RT-qPCR) and BALF Assays

Total RNA was extracted from snap-frozen lung tissue with a RNeasy kit (Qiagen, Germany) and converted to cDNA with a High Capacity cDNA Kit (Thermofisher Scientific, US). qPCR was performed using bioinformatically validated TaqMan assays (Thermofisher Scientific, US). The threshold cycle values (Ct) of target genes were normalized to a reference gene (glyceraldehyde phosphate dehydrogenase—Gapdh) and the relative fold change was calculated using the ΔΔCt method. To measure markers of neutrophil extracellular traps (NETs), myeloperoxidase (MPO) activity in the BALF was determined using o-Dianisidine substrate (Sigma-Aldrich, US) and dsDNA levels were quantified using a Quant-iT™ PicoGreen™ dsDNA Assay (Thermofisher Scientific, US). To assess lung injury, cell death marker LDH was determined in the BALF using a Cytotoxicity Detection Kit (Sigma-Aldrich, US) and total protein contents were measured using a BCA Protein Assay Kit (Thermofisher Scientific, US).

### Data Analysis

All data were statistically analysed using GraphPad Prism 9.0 (GraphPad, US). Animal data were presented as the mean±SEM. Where detailed and appropriate, unpaired Student's t-tests, one-way ANOVA with Sidak's post-hoc tests, or Pearson's correlation were performed. Non-parametric data from the clinical samples were reported as the median±interquartile ranges and analysed by the Mann-Whitney test or Spearman's correlation. p<0.05 was considered to be statistically significant.

### Example 2: Expression of the βc Receptor CSF2RB is Increased in the Lungs of Severe Asthmatics

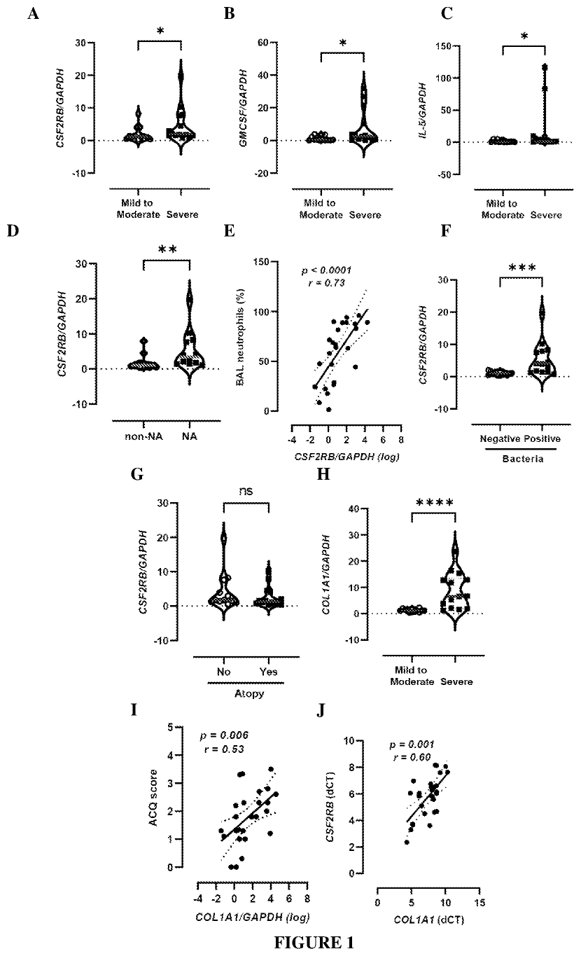

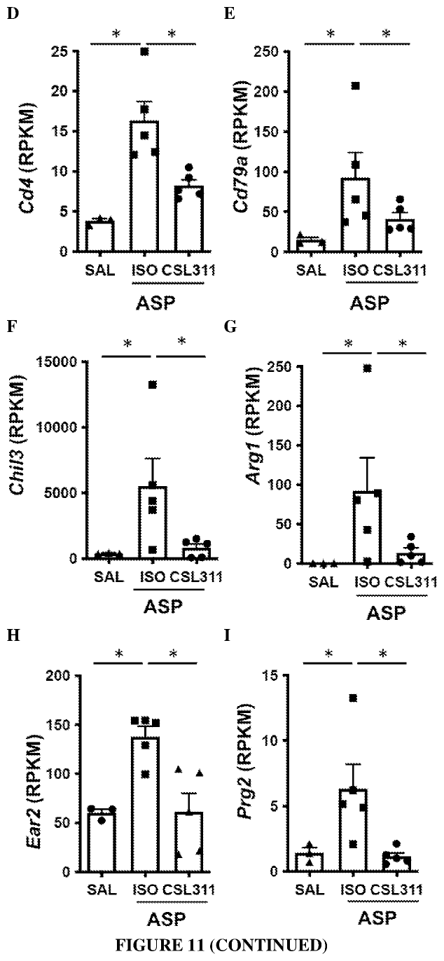

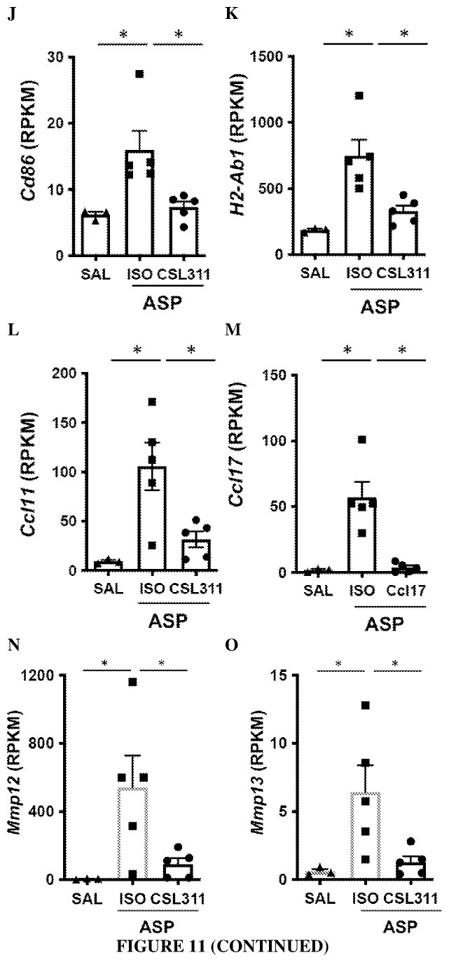

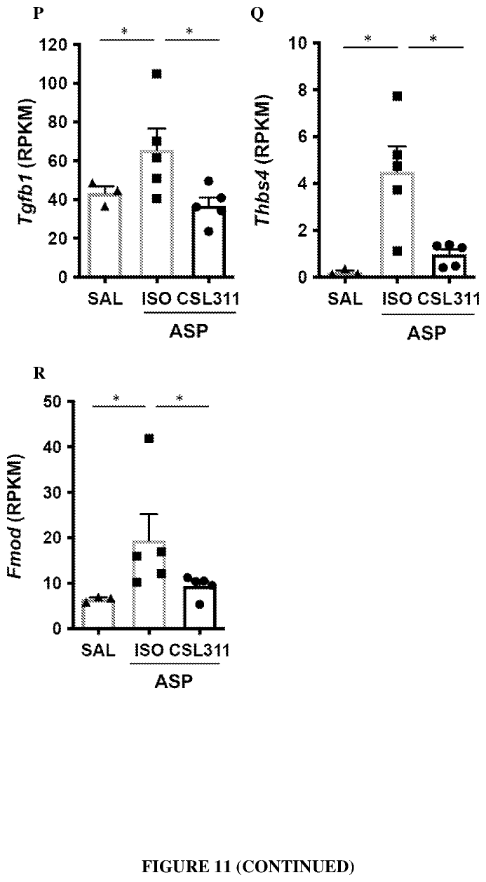

The gene expression of βc receptor CSF2RB, as well as GMCSF, IL5 and IL3 was quantified in bronchial biopsies from a cohort of asthmatic patients grouped by disease severity as previously described (Wang H et al., J Allergy Clin Immunol 2019; 143:785-8 e6). Severe asthmatics showed a 2-fold increase in CSF2RB expression compared to patients with mild-to-moderate disease (FIG. 1A, p<0.05). GMCSF and IL5 transcripts were increased by 5-fold and 3-fold respectively (FIGS. 1B and C, both p<0.05) while IL3 was below the limit of detection. In addition, neutrophilic asthmatics (NA) had 3-fold higher CSF2RB levels compared to non-neutrophilic asthmatics (non-NA) (FIG. 1D, p<0.01), the level of which strongly correlated with their BAL neutrophil percentages (FIG. 1E, p<0.0001). When patients were further stratified based on the presence of bacteria in airways (FIG. 1F) or atopy (FIG. 1G), CSF2RB levels were significantly increased in asthmatics with bacteria (3-fold, p<0.0001) but not in asthmatics with atopy. Expression of pro-collagen gene COL1A1 was analysed, where there was a 6-fold increase in patients with severe asthma compared to those with mild-to-moderate disease (FIG. 1H, p<0.0001). Of significance, high COL1A1 expression positively correlated with both worse asthma symptoms (ACQ score) and high CSF2RB expression (FIGS. 11 and 1J, both p<0.01).

### Example 3: Lung Inflammation and CSF2RB Expression in a Pre-Clinical Model of Severe Asthma in hβcTg Mice

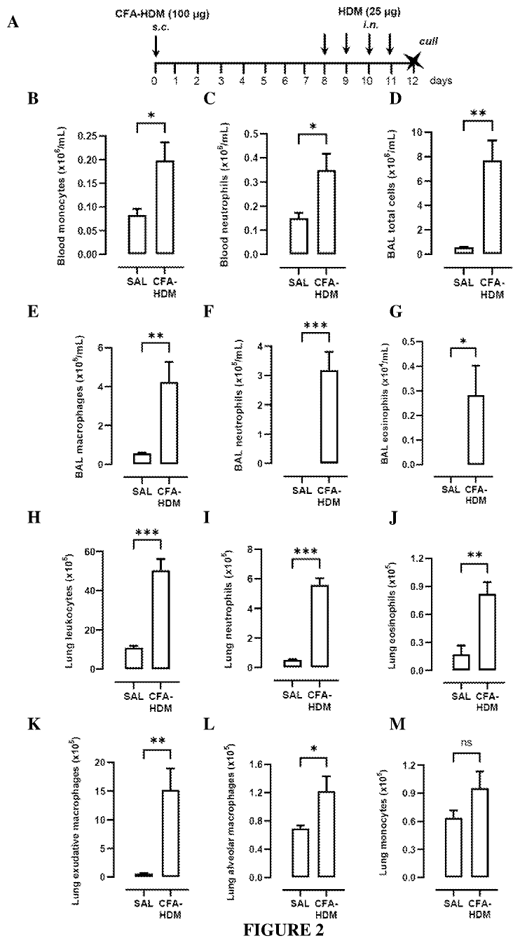

CSF2RB gene expression in an established pre-clinical model of severe asthma (previously described in Ouyang S et al., JCI Insight 2020) using hβcTg mice was evaluated as summarised in FIG. 2A. This CFA-HDM protocol induced a systemic inflammatory response with high numbers of circulating blood monocytes (FIG. 2B) and neutrophils (FIG. 2C) being detected in hβcTg mice, which was accompanied with BAL inflammation (FIG. 2D) dominated by infiltrating macrophages (FIG. 2E), neutrophils (FIG. 2F) and eosinophils (FIG. 2G). In lung tissue, marked leukocyte (CD45⁺) infiltration was observed in CFA-HDM treated mice (FIG. 2H), with significant increases in recruited neutrophils (CD45⁺Ly6G^{high}CD11b⁺, FIG. 2I) and eosinophils (CD45⁺F4/80⁺CD11c^{low}SiglecF^{high}, FIG. 2J). The predominant inflammatory cell type was CD11b⁺ monocyte-derived exudative macrophages (CD45⁺F4/80⁺CD11c⁺CD11b⁺, FIG. 2K), accounting for >35% of all infiltrated cells. Alveolar macrophages (CD45⁺F4/80⁺CD11c^{high}SiglecF^{high}, FIG. 2L) were also increased while monocytes (CD45⁺F4/80⁺CD11c⁻CD11b⁺, FIG. 2M) were unaltered at this time-point.

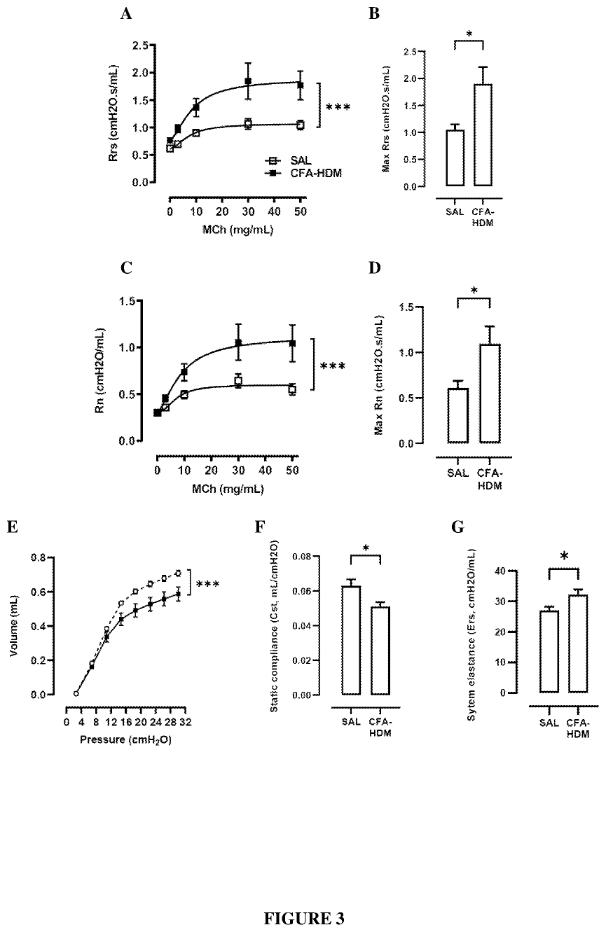

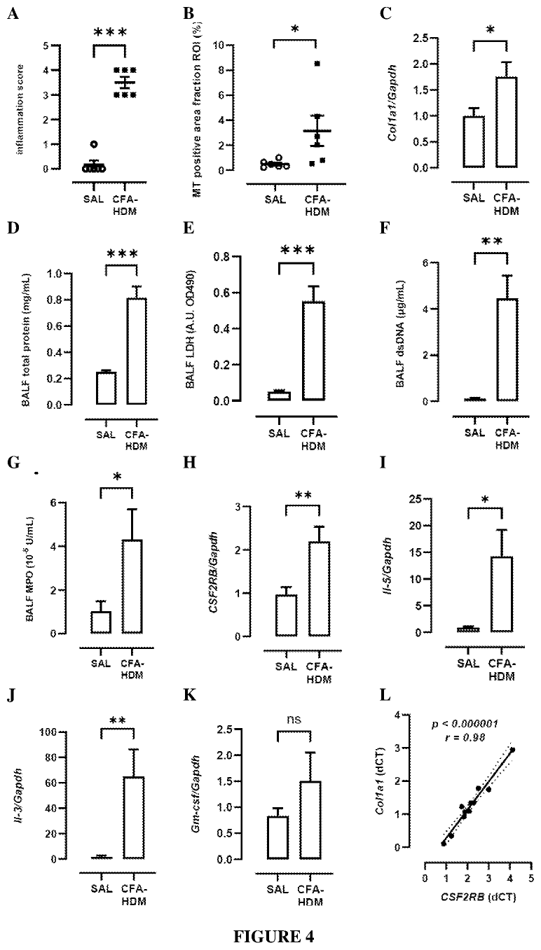

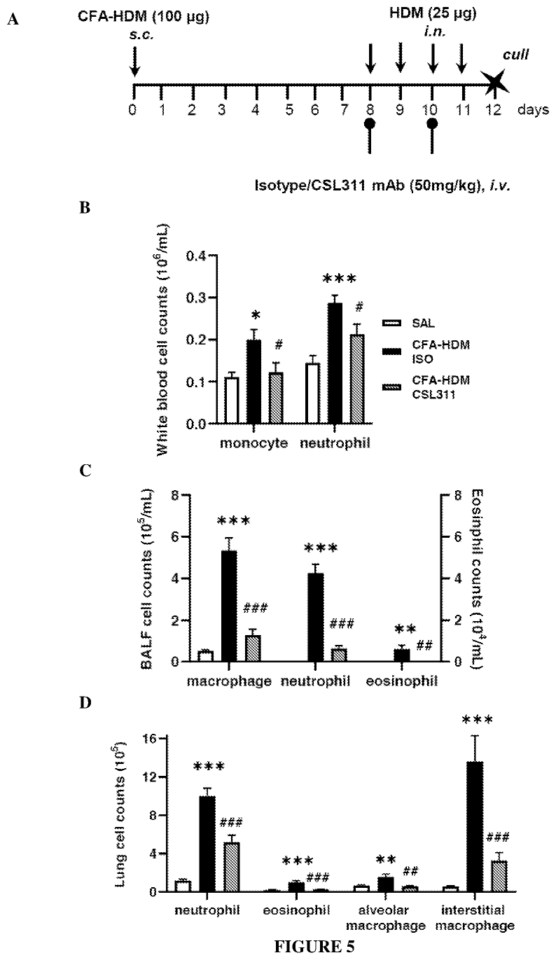

The respiratory mechanics of the CFA-HDM treated hβcTg mice was also evaluated. Nebulised Mch dose-dependently increased total respiratory resistance (Rrs, FIG. 3A) and airway resistance (Rn, FIG. 3C) in hβcTg mice, and this response was potentiated by CFA-HDM treatment. Significant increases in maximal Rrs and Rn were subsequently detected in CFA-HDM treated mice (FIGS. 3B and 3D). At baseline, CFA-HDM treatment led to a downward shift in the pressure-volume curve (FIG. 3E), as well as reduction in respiratory compliance (Cst, FIG. 3F) and increase in system elastance (Ers, FIG. 3G), demonstrating a restrictive respiratory pattern that is suggestive of tissue stiffness and pulmonary fibrosis. Underlying these functional alternations, marked histopathology associated with peribronchiolar and alveolar inflammation was detected in CFA-HDM treated mice (FIG. 4A). MT-stained lung tissue sections also identified larger regions of intense collagen staining around their airways (FIG. 4B). A significant increase in the expression of pro-type I collagen gene (Col1a1, FIG. 4C) further supported the establishment of pulmonary fibrosis in CFA-HDM treated mice. Markers of lung injury and oedema were also detected, measured by the increase in total BALF protein and BALF LDH in CFA-HDM treated mice (FIGS. 4D and E). In addition, markers of neutrophil extracellular traps (NETs)—dsDNA and MPO (FIGS. 4F and 4G), were concurrently increased in the BALF. Assessment of CSF2RB, Il-5, Il-3 and Gm-csf lung expression in CFA-HDM treated mice revealed a 2-fold increase of CSF2RB (FIG. 4H, p<0.01), 14-fold increase of Il-5 (FIG. 4I, p<0.01) and 60-fold increase of Il-3 (FIG. 4J, p<0.01), whereas Gm-csf was not significantly altered at this timepoint (FIG. 4K). There was a strong positive correlation between CSF2RB and Col1a1 expression in hβcTg mice (FIG. 4L).

### Example 4: Severe Asthma in hβcTg Mice is Blocked by CSL311 Antibody Treatment

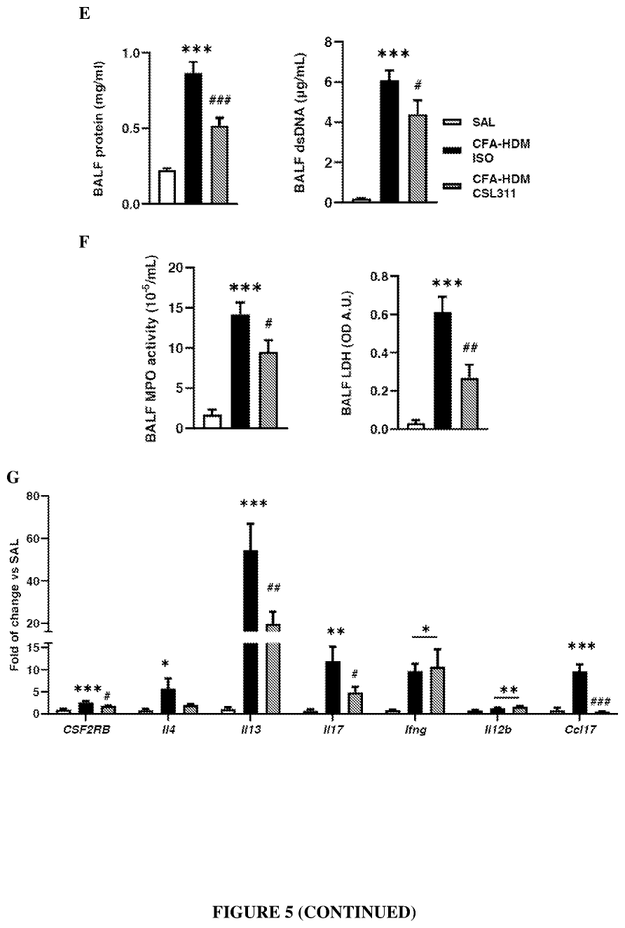

CSL311 was then administered to hβcTg mice during the HDM challenge phase to evaluate its therapeutic potential (FIG. 5A). In CFA-HDM treated mice, CSL311 fully prevented the expansion of blood monocytes and neutrophils (FIG. 5B, p<0.05) and significantly reduced the BAL and lung infiltration of all types of leukocytes measured (FIGS. 5C and D, p<0.001). Differential cell analysis revealed that there was a 76% reduction in BAL macrophages, 85% reduction in BAL neutrophils and near complete diminishment of BAL eosinophils with CSL311 treatment (FIG. 5C, all p<0.01). In lung tissue, CSL311 significantly lowered the infiltration of total leukocytes by 50%. Lung neutrophils and interstitial macrophages were reduced by 50% and 76% respectively, and lung eosinophils and alveolar macrophages were reduced to control levels (FIG. 5D, all p<0.01).

In CFA-HDM_ISO treated mice, makers of neutrophil extracellular traps (NETs) including dsDNA and MPO activity and markers of lung injury such as BALF protein and LDH were detected, all of which were reduced by CSL311 (FIGS. 5E and 5F). Assessment of CSF2RB lung expression in CFA-HDM_ISO treated mice further revealed a 2-fold increase of CSF2RB, which was lowered to control levels by the blockade of βc with CSL311 (FIG. 5G). Th2/Th17 cytokines Il4, Il13 and Il17 were induced by CFA-HDM and suppressed by CSL311 whilst Th1 cytokines Ifng and Il12b induced by CFA-HDM were preserved in CSL311 treated mice (FIG. 5G). T cell chemokine Ccl17 was induced by CFA-HDM and suppressed by CSL311 (FIG. 5G).

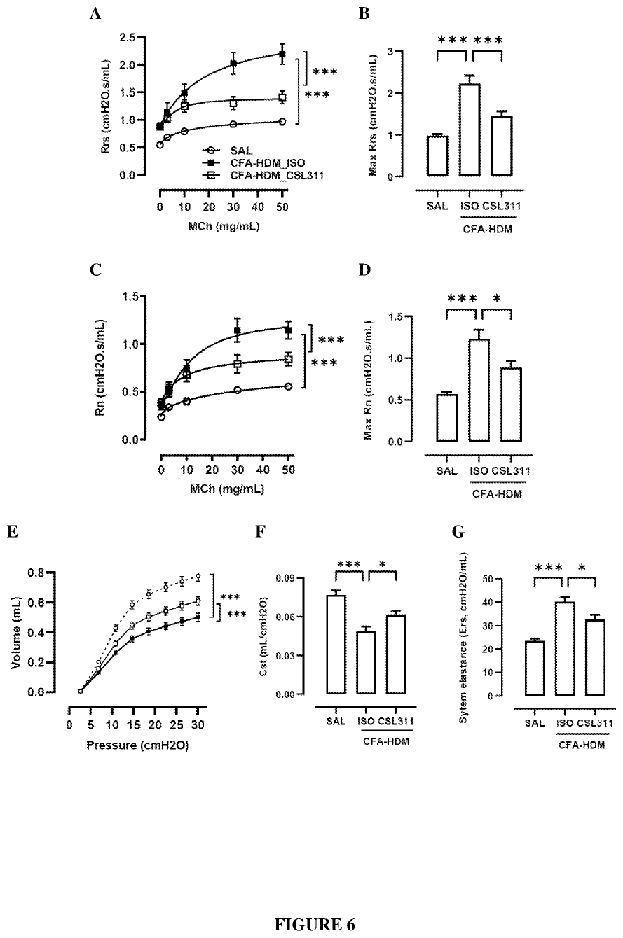

Whether this potent anti-inflammatory response mediated by CSL311 translated to improved lung function in CFA-HDM treated mice was then explored. Of importance, respiratory resistance including Rrs and Rn in response to the increasing doses of Mch was reduced in CSL311 treated mice (FIGS. 6A and 6C, p<0.0001) and maximal responses in Rrs and Rn were significantly decreased (FIGS. 6B and 6D). In addition, the baseline airway constriction reflected by the shifted pressure-volume curve (FIG. 6E), reduced Cst (FIG. 6F) and increased Ers (FIG. 6G) were improved with CSL311 treatment (p<0.05).

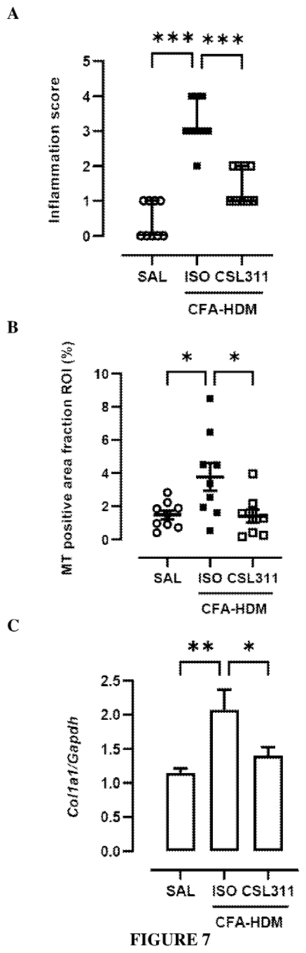

Histopathology including alveolar and peribronchiolar inflammation was significantly reduced (FIG. 7A, p<0.001), as was airway fibrosis measured by quantifying Masson's Trichrome (MT)-stained sections (FIG. 7B, p<0.05). Consistent with the MT-staining, lung expression of Col1a1 (FIG. 7C) was reduced to control levels.

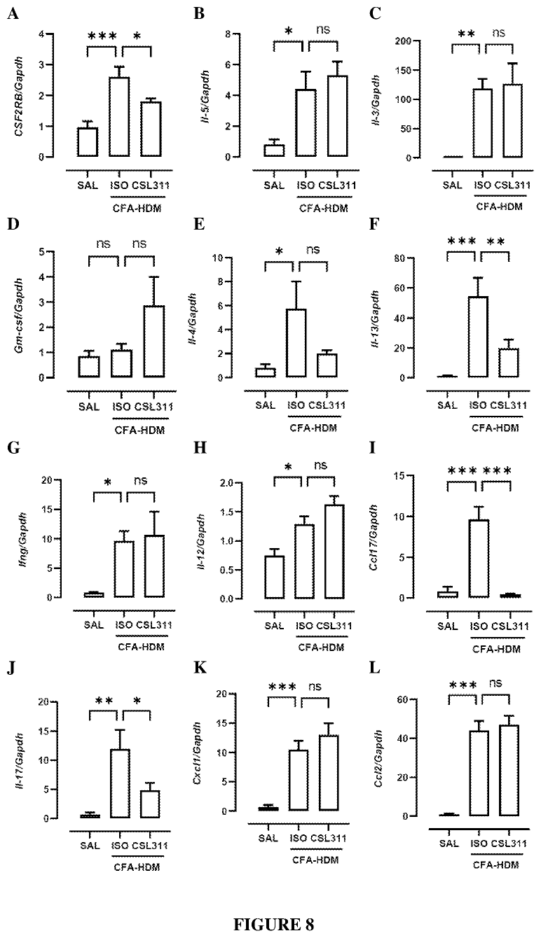

At a molecular level, blockade of βc with CSL311 reduced lung expression of CSF2RB in CFA-HDM treated mice (FIG. 8A, p<0.05), whereas βc cytokine expression was not significantly altered (FIGS. 8B, 8C and 8D). In CFA-HDM treated mice, Th2 cytokines Il-4 and Il-13 were increased, and both were reduced by CSL311 treatment, although only Il-13 reached statistical significance (FIGS. 8E and 8F). The expression of Th1 cytokines Ifng and Il-12 were induced in the lungs of CFA-HDM treated mice and CSL311 treatment did not alter Th1 cytokine expression (FIGS. 8G and 8H). The Th2-biased inhibitory profile of CSL311 was consistent with its suppression of Ccl17, a Th2 cell chemokine that was highly elevated in our model and effectively reduced by βc blockade with CSL311 (FIG. 8I). The Il-17 cytokine and neutrophil chemokine Cxcl1 were increased in CFA-HDM treated mice and only Il-17 was reduced by CSL311 (FIGS. 8J and 8K). Monocyte/macrophage chemokine Ccl2 was increased in these mice, which was not affected by CSL311 treatment (FIG. 8L).

### Example 5: Blocking βc Signaling Inhibits Loss of Airway Function and Development of Airway Fibrosis Following 5 Weeks of Intranasal House Dust Mite Extract Exposure

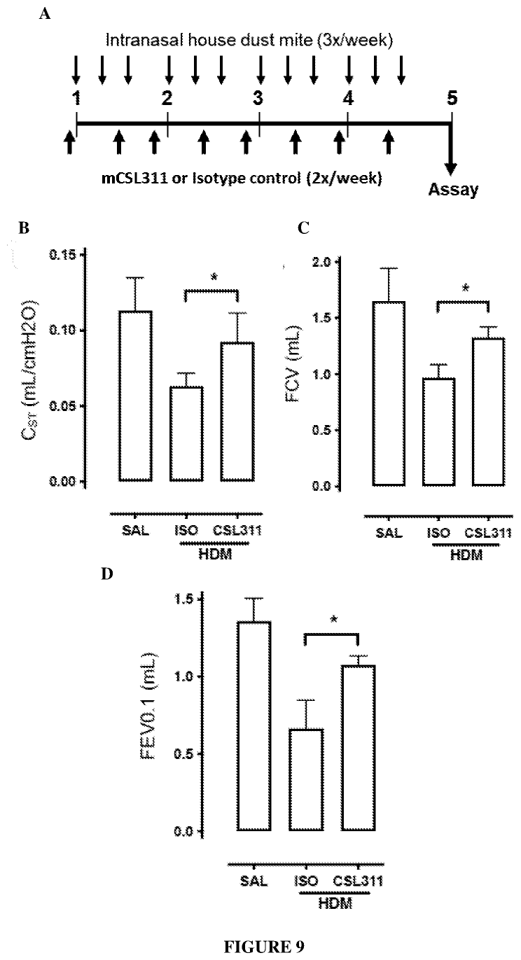

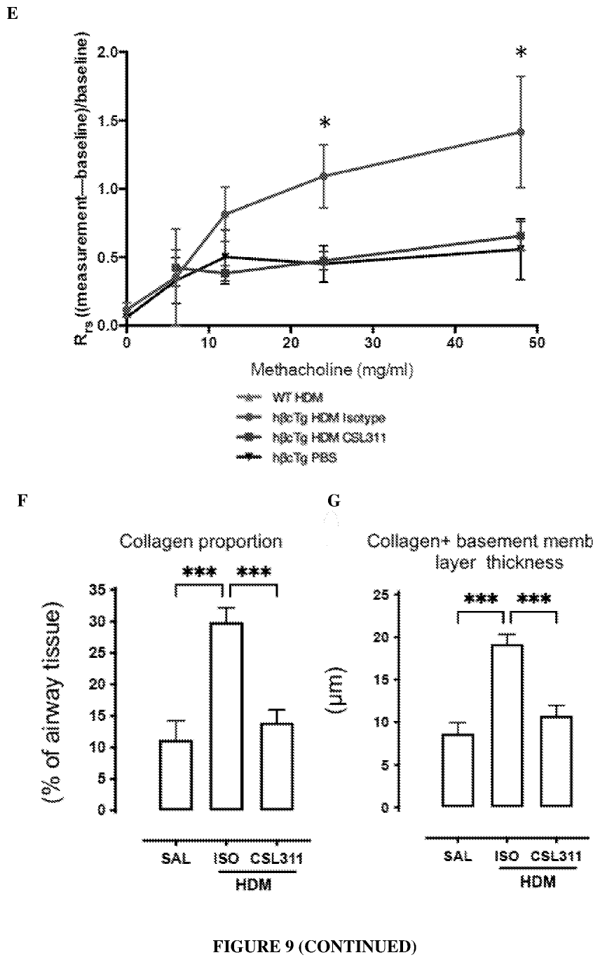

To determine if CSL311 could prevent AHR and airway fibrosis in response to chronic allergen exposure, hβcTg mice were challenged intranasally (i.n.) with 25 ul of 50 mg/ml of a house dust mite every second day for 5 weeks, during which CSL311 or isotype control antibodies were administered twice a week (FIG. 9A). 48 hours after the last HDM challenge airway function and AHR measurement was done and the lungs were removed, fixed, embedded, sectioned and stained with Masson's Trichrome stain.

Chronic HDM challenge led to reductions in CST and FVC that occur in airways with fibrosis and reduction in FEV0.1 that occurs in lungs with narrowed airways, all of which were improved (approximately 50% rescue) by CSL311 (FIG. 9B-D). HDM challenge resulted in significantly increased AHR in hβcTg mice which was completely blocked by CSL311 (FIG. 9E). Analysis of collagen using MT staining showed that administration of HDM for 5 weeks induced significant collagen deposition around the airways and that treatment of hβcTg mice with CSL311 significantly reduced the amount of collagen (FIG. 9F). Moreover, the thickness of the subepithelial area of the airways was significantly reduced in CSL311-treated hβcTg mice (FIG. 9G).

### Example 6: Blocking βc Signaling Inhibits Inflammation, Loss of Airway Function and Airway Remodelling Following 8 Weeks of Intranasal Aspergillus Extract Exposure

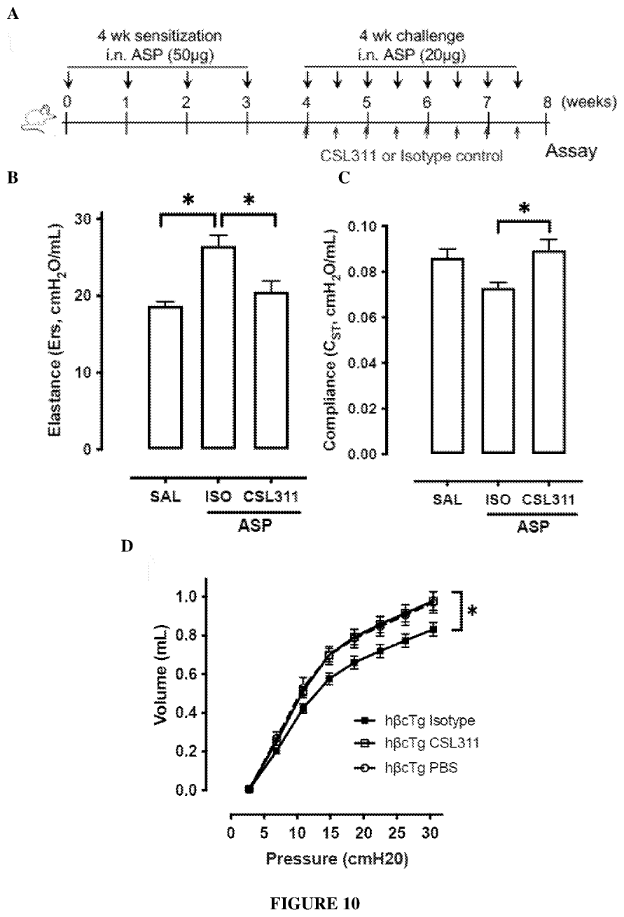

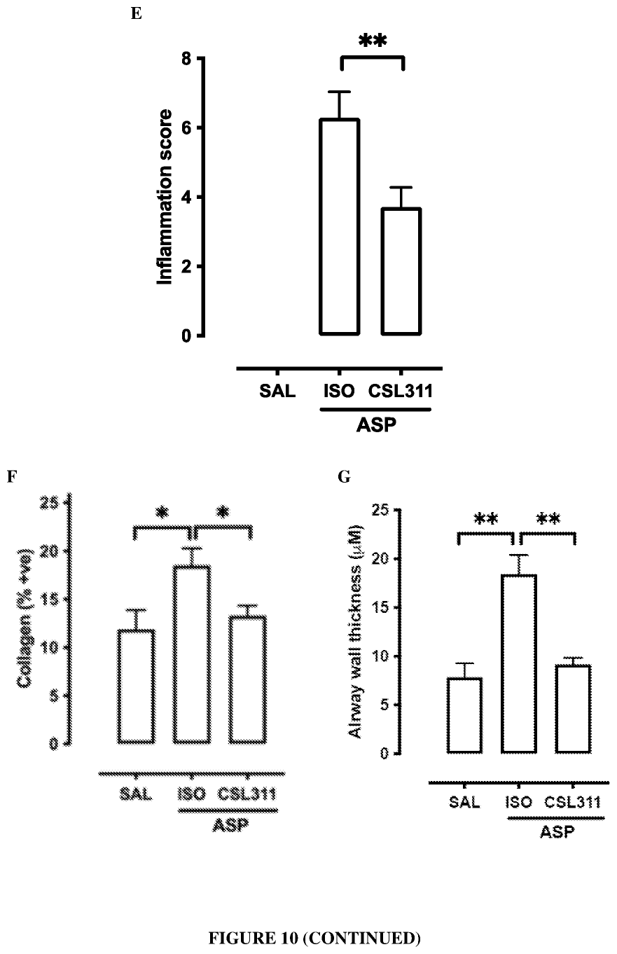

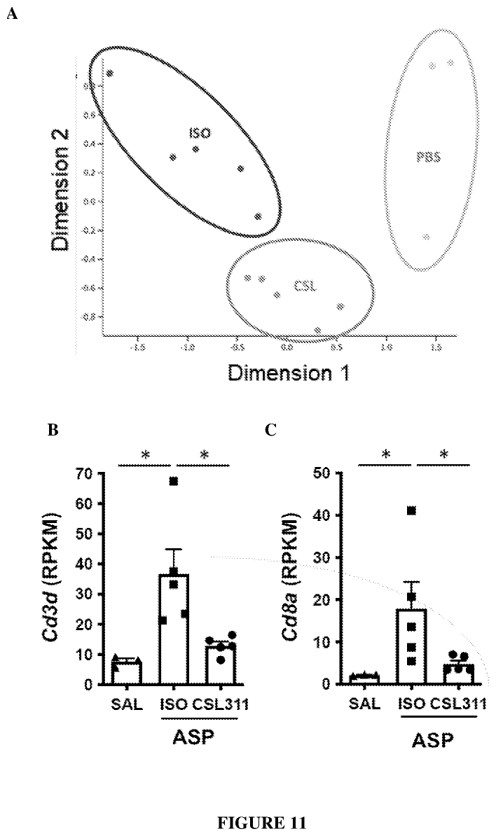

Mice were treated intranasally with 50 ug Aspergillus (Asp; extract from Aspergillus Fumigatus that was purchased from Stallergenes Greer (item XPM3D3A25, lot 365084)) once a week for 4 weeks followed by 20 ug Asp every second day for 4 weeks. Isotype and CSL311 groups receive intravenous injection of antibody via the tail vein for the final 4 weeks (FIG. 10A). Chronic Aspergillus extract exposure in this model induces airway remodelling and a mixed lymphocytic and granulocytic infiltrate into the lungs that includes eosinophils and neutrophils (Ichikawa et al., 2019, Nat. Immunol. 20(11):1469-80). Lung function was measured using a Flexivent (Scireq). Exposure to ASP induced fibrosis-associated changes in lung function including increased elastance (Ers), CST (FIG. 10B-C), and associated pressure volume curves (FIG. 10D). In contrast to the HDM challenged model there was no change in FVC or FEV0.1 following ASP treatment. Nonetheless, CSL311 was able to rescue each of these changes returning their values to those similar to saline treated animals (FIG. 10B-D). In addition, tissue inflammation was induced by ASP as previously described (Ichikawa et al., 2019, Nat. Immunol. 20(11):1469-80) and was significantly inhibited by CSL311 (FIG. 10F). Visual changes to the airway using microscopy analysis of Masson's trichrome staining of lung sections was assessed. Markers of airway remodelling, collagen content around the airways and the thickness of the subepithelial layer of the airways, were increased by ASP treatment (data not shown). Importantly, both collagen (FIG. 4G) and subepithelial thickness (FIG. 4I), were strongly inhibited by treatment of the mice with CSL311.

### Example 7: Blocking βc Signaling with CSL311 Normalisation of Gene Expression and Extracellular Matrisome in ASP Challenged Mice

RNA-Sequencing on lung tissue from saline-challenged and ASP-challenged mice treated with isotype control antibody or CSL311 was performed. Gene expression in saline treated control hβcTg mice (n=3), ASP-treated Isotype-treated hβcTg mice (n=5) and ASP-treated CSL311-treated hβcTg mice (n=5) was analysed. Representation of the data using multi dimensional scaling (MDS) showed that the largest amount of variance is as a result of the ASP challenge with a smaller amount of variance attributed to the CSL311 intervention (FIG. 11A). Importantly, each group clusters away from each other. ASP treatment resulted differential regulation of 1043 genes with 728 genes upregulated and 315 genes downregulated. Administration of i.v. CSL311 to ASP treated mice resulted in 459 genes differentially regulated, with the majority (432 genes) downregulated. Of the 728 genes upregulated by ASP treatment 296 were downregulated by CSL311 treatment indicating that CSL311 broadly inhibits ASP associated changes in gene expression. Of the ASP upregulated genes that were downregulated by CSL311, the most enriched KEGG pathway was Cytokine-Cytokine Receptor Interaction (31 genes, p=1.3×10⁻¹⁴). The top biological process was immune system process (50 genes, p=4.2×10⁻²⁷) followed by adaptive immune response (35 genes, p=2.9×10⁻²³) and positive regulation of T cell activation (13 genes, p=3.4×10⁻¹⁴). The genes enriched in these biological processes consisted primarily of those encoding surface receptors including Cd3d, Cd4 and Cd8a that are expressed on T cells and the accessory molecules Cd79a that is expressed on B cells, several MCH-II genes, the antigen-presenting cell-expressed co-stimulatory molecule CD86. Of note, Arg1 and Chil3, markers of M2 macrophages and Ear2 and Prg2 which are specifically expressed by eosinophils were also decreased by CSL311 treatment. Notably, Ccr4 that is highly expressed by Th2 cells and Th17 cells in single-cell RNAseqence analysis was increased by ASP and reduced by CSL311. The same pattern of expression on Ccl17, a CCR4 ligand was also observed. Selected immune genes are shown in FIG. 11B-M. Notably, prediction of cell types using xCell analysis indicated that Th2 cells and dendritic cells were decreased following CSL311 treatment. Gene set enrichment analysis further demonstrated the marked dependency of be signalling in inflammation following ASP exposure with strong enrichment of the hallmark inflammatory response gene set in the genes downregulated by CSL311 treatment (data not shown). To dissect the inflammatory response further ingenuity pathway analysis (IPA) was used to identify gene modules that were inhibited by CSL311. Broad inhibition of Th1, Th2 signatures and IL-17 signalling along with decreases in genes associated with myeloid cell recruitment was observed.

Analysis of lung tissue and airway function following ASP challenge indicated that blocking βc resulted in strong inhibition of markers of airway remodelling including increased amounts of collagen around the airways and thickening of the airway wall and changes in airflow indicative of loss of airway tissue compliance. Abnormal extracellular matrix deposition is one of the main pathological processes that results in tissue remodelling and fibrosis.

To investigate how CSL311 protects against airway remodelling, the components of the extracellular matrix which were altered in response to ASP exposure and CSL311 treatment were identified. The publicly available Matrisome database is a comprehensive catalogue of extracellular matrix components and extracellular matrix-associated genes that was used to define the effect of ASP challenge and CSL311 on extracellular matrix (ECM) formation. Using IPA analysis with the matrisome database gene list showed the matrisome gene signature was significantly reduced by CSL311 and that the effect was most evident for ECM regulators and matrisome associated genes (data not shown). GSEA also showed that both ECM regulators and matrisome-associated genes were strongly enriched in the CSL311-downregulated gene list (data not shown). 24 ECM components were upregulated by ASP treatment, of which 14 were reduced by CSL311. These included important fibrosis-associated genes including Thromobospondin 4 (Thbs4), Fibromodulin (Fmod), other genes included Podnl1, Mfge8 and Slamf6. Moreover, of the 744 known ECM components and ECM regulators in the matrisome database ˜8% were upregulated (59 genes) and ˜8% were down regulated (64 genes) and in each case the majority of these were significantly rescued (toward levels detected in saline challenged mice) by treatment with CSL311. Unsupervised hierarchical clustering of these genes visually shows that the ASP-induced ECM gene program is largely normalized toward that similar to unchallenged animals following treatment with CSL311 (data not shown) with both ASP-upregulated and ASP-downregulated returned to levels more similar to saline treated animals. Selected ECM genes increased by ASP exposure and reduced by CSL311 are shown in FIG. 11N-R. Together these data indicate that blocking βc cytokine signalling results in reprogramming to a healthy airway following chronic ASP exposure.

### Example 8: Airway Hyper-Responsiveness and Severe Emphysema are Reduced by CSL311 Treatment

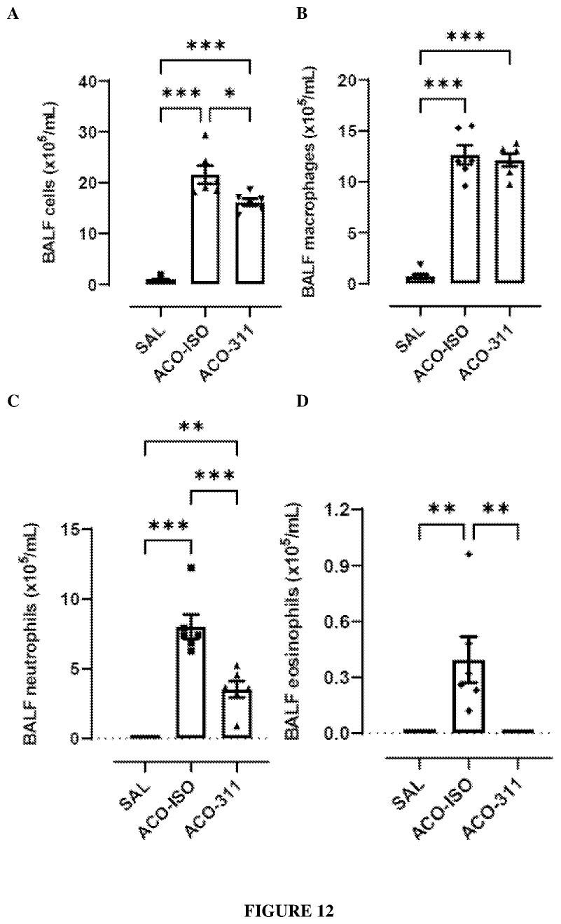

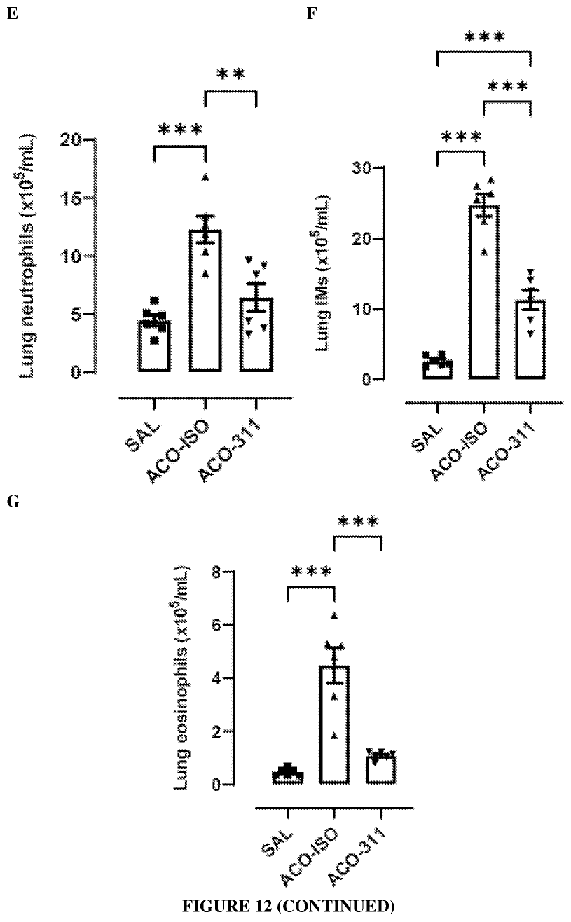

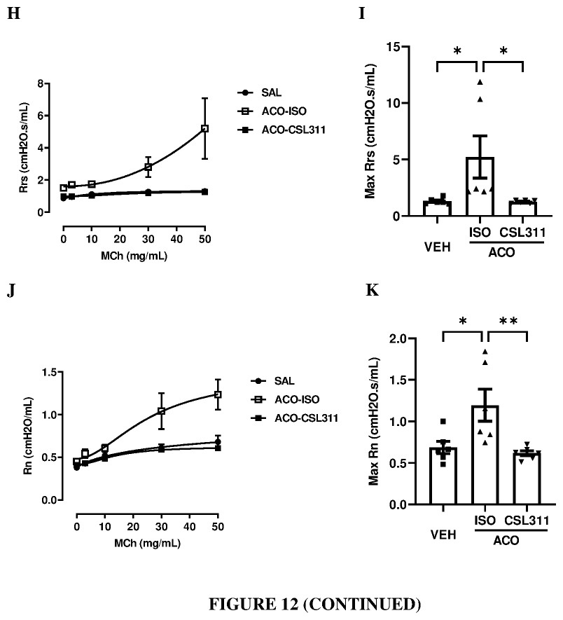

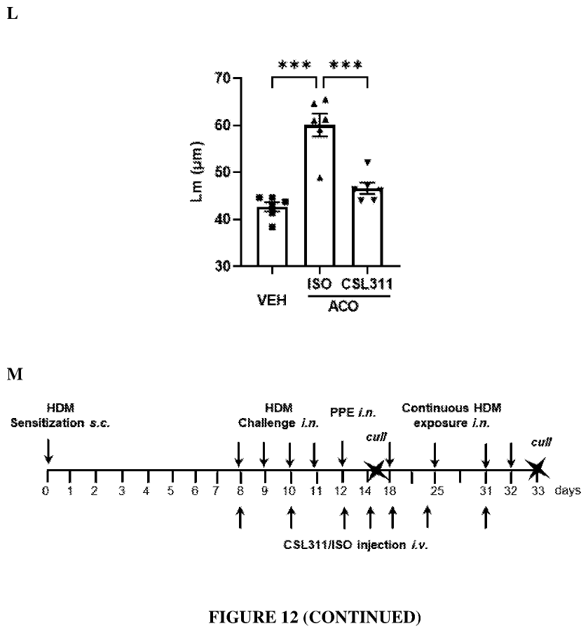

To investigate eosinophil-associated COPD/asthma overlap (ACO), female hβcTg mice were sensitised (s.c. injection) with house dust mite extract (HDM, Dermatophagoides pteronyssinus, Greer Laboratories, US; 100 g emulsified in complete Freund's adjuvant, CFA) and further challenged with porcine pancreatic elastase (PPE, 0.5 U in saline, i.n.) and intranasal HDM (25 g in saline) as shown in FIG. 12M. CSL311 or isotype control was administered to the HDM+PPE co-treated hβcTg mice (ACO) intravenously (i.v.) at 50 mg/kg on experimental day 8, day 10, day 12, day 14, day 18, day 25, and day 31, respectively. Mice were euthanized on experimental day 15 to assess the lung inflammation, or day 33 for lung function measurements and lung histology.

During the inflammatory phase (day 15), infiltration of immune cells to the BAL compartment (FIG. 12A), comprised macrophages (FIG. 12B), neutrophils (FIG. 12C) and eosinophils (FIG. 12D) was markedly increased in ACO mice. Pc antagonism by CSL311 significantly reduced BALF neutrophils (FIG. 12C) and eosinophils (FIG. 12D). In the lung compartment, accumulation of neutrophils (FIG. 12E), CD11b+ macrophages (including IMs and EMs, FIG. 10F) and eosinophils (FIG. 12G) was increased. βc antagonism by CSL311 largely reduced the lung accumulation of all these immune cell types. On experimental day 33, airway hyper-responsiveness (AHR) measured by increased total respiratory resistance (Rrs, FIGS. 12H and I) and airway resistance (Rn, FIGS. 12J and K)) in ACO mice was also prevented by CSL311. At a histopathological level, airspace enlargement or emphysema measured by the increased mean linear intercept on H&E stain lung sections (Lm, FIG. 12L) was fully prevented by CSL311.

### Example 9: Human, Non-Interventional Analysis of CD131 as a Target for Reducing Inflammation in Chronic Airway Disease

Peripheral blood (PB) and sputum was collected from 50 asthmatics and 30 COPD patients. PB was also collected from 60 matched healthy volunteers (HV). RNA was extracted and analysed by RNA sequencing. Immune cell subsets were quantified, and baseline activation and receptor expression were assessed by flow cytometry or Wright's staining of sputum Cytospins. White blood cells (WBC) were isolated and stimulated with combined βc cytokines+/− CSL311, and activation was evaluated by flow cytometry. Serum protein levels were measured by Luminex assays.

CD131 was detected on key immune cell effectors and expression was maintained in disease. Several subsets were elevated in PB of patients with airway disease and these cells exhibited a more activated profile (Table 2). Airway infiltration of neutrophils and eosinophils was also demonstrated by sputum cell counts.

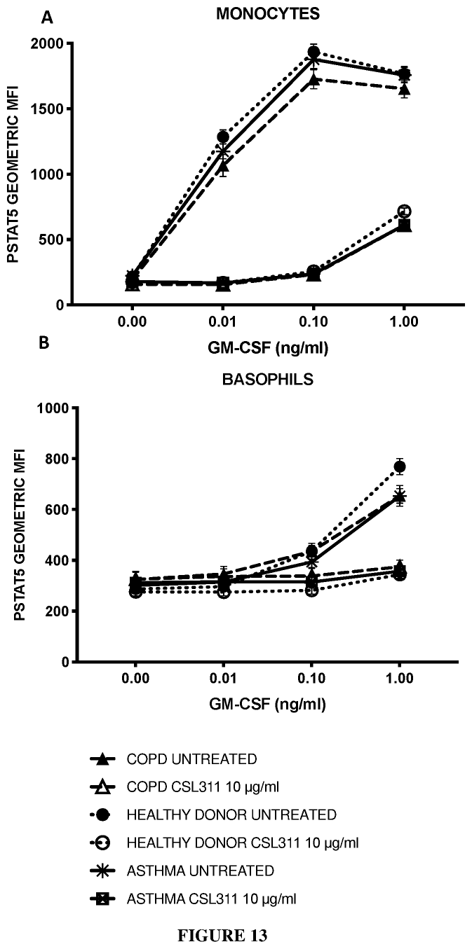

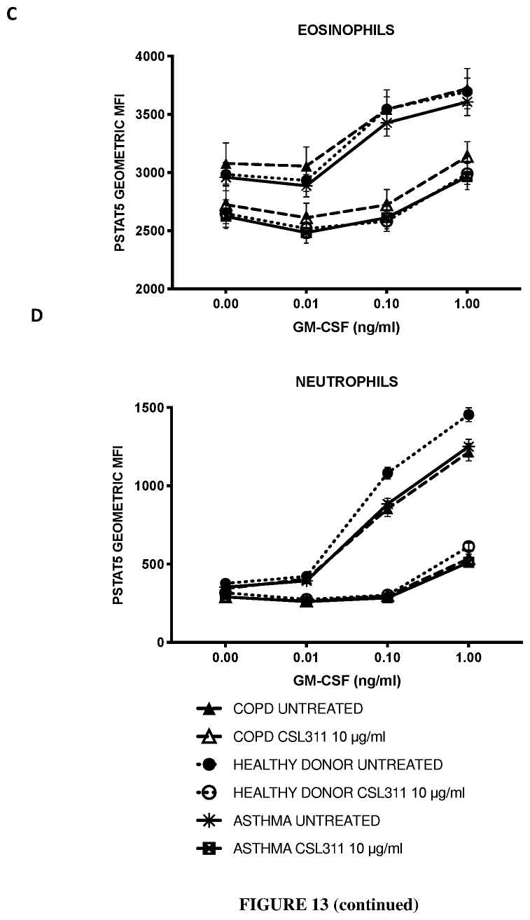

Induction of CD11b and CD35 expression and CD62L shedding was replicated ex-vivo by stimulating WBC with βc-cytokines IL-3, IL-5 and GM-CSF. As shown in Table 3, CSL311 pre-treatment was able to inhibit this response. In addition, GM-CSF-induced STAT5 activation was inhibited by CSL311 in a dose-dependent manner (FIG. 13).

Pathway analysis of the transcriptomic data derived from peripheral blood cells indicated over representation of transcripts related to inflammatory responses and immune cell trafficking in the peripheral blood from asthma and COPD patients.

Evidence of additional βc-cytokine activity was seen systemically, with serum CCL13 significantly elevated in asthmatics (p<0.0001) and CCL17 elevated in both asthma and COPD patients when compared to the matched healthy volunteers (p<0.001, p=0.011 respectively).

TABLE 2
CD131 expression and changes in peripheral blood cell frequency and activation in asthma and COPD

| | HV | ASTHMA | | | | | |
|---|---|---|---|---|---|---|---|
| CELL TYPE | CD131+ | CD131+ | Count | CD11b | CD35 | CD62L | CD63 |
| BASOPHIL | Yes | Yes | ↑ | ↑ | — | ↓ | ↓ |
| | | — | p = 0.0382 | p = 0.0020 | | p = 0.0014 | p = 0.0006 |
| EOSINOPHIL | Yes | Yes | ↑ | ↑ | — | ↓ | ↓ |
| | | ↑ | p = 0.0017 | p = 0.0001 | | p = 0.0011 | p = 0.0116 |
| | | p = 0.0091 | | | | | |
| MONOCYTE | Yes | Yes | — | ↑ | ↑ | — | ↓ |
| | | — | | p = 0.0063 | (p = 0.0247) | | |
| NEUTROPHIL | Yes | Yes | ↑ | ↑ | ↑ | ↓ | ↓ |
| | | — | p = 0.0023 | p < 0.0001 | (p = 0.0332) | p = 0.0015 | |

| | COPD | | | | | |
|---|---|---|---|---|---|---|
| CELL TYPE | CD131+ | Count | CD11b | CD35 | CD62L | CD63 |
| BASOPHIL | Yes | — | — | — | — | ↓ |
| | — | | | | | p = 0.0005 |
| EOSINOPHIL | Yes | — | ↑ | — | — | ↓ |
| | — | | p < 0.0001 | | | p = 0.0073 |
| MONOCYTE | Yes | — | — | — | — | ↓ |
| | — | | | | | p = 0.0004 |
| NEUTROPHIL | Yes | ↑ | — | — | — | ↓ |
| | — | p = 0.001 | | | | p = 0.0046 |

Yes indicates CD131 expression above 200 ABC (antibodies bound per cell); ↑ indicates significant increase compared to healthy volunteers (HV); ↓ indicates significant decrease compared to HV; — indicates no significant change.

TABLE 3
Ex vivo stimulation of white blood cells and effect of CSL311 pre-treatment compared to healthy volunteers

| | ASTHMA | | | | COPD | | | |
|---|---|---|---|---|---|---|---|---|
| | Basophils | Eosinophils | Neutrophils | Monocytes | Basophils | Eosinophils | Neutrophils | Monocytes |
| CD35 | Increased | Increased | Increased | — | Increased | Increased | Increased | — |
| Modulated by CSL311 | Yes | Yes | Yes | | Yes | Yes | Yes | |
| CD62L | — | Decreased | Decreased | Decreased | — | Decreased | Decreased | |
| Modulated by CSL311 | | Yes | Yes | | | Yes | Yes | |
| CD11b | Increased | Increased | Increased | — | Increased | Increased | Increased | — |
| Modulated by CSL311 | Yes | Yes | Yes | | Yes | Yes | Yes | |

WB markers analysed used a paired two-tailed t-test.
The ability of IL-3/IL-5/GM-CSF to activate the cells was determined for the stimuli that induced >10% change in MFI and tested by uncorrected fisher's LSD test.
The ability of CSL311 to modulate this activation was then tested for the stimuli that had >5% change in MFI and tested with a paired two-tailed t-test.
“—” no significant difference detected.

1. A method of treating severe asthma in a subject, the method comprising administering to the subject a compound that binds to or specifically binds to CD131 and neutralises signaling by interleukin (IL) 5 and granulocyte-macrophage colony stimulating factor (GM-CSF) in a subject.

2. A method of depleting and/or reducing eosinophils and/or neutrophils in a subject suffering from severe asthma, the method comprising administering to the subject a compound that binds to or specifically binds to CD131 and neutralises signaling by interleukin (IL) 5 and granulocyte-macrophage colony stimulating factor (GM-CSF) in a subject.

3. A method of treating neutrophilia and/or eosinophilia in a subject suffering from severe asthma, the method comprising administering to the subject a compound that binds to or specifically binds to CD131 and neutralises signaling by interleukin (IL) 5 and granulocyte-macrophage colony stimulating factor (GM-CSF) in a subject.

4. The method of any one of claims 1 to 3, wherein the severe asthma is difficult-to-treat asthma, treatment resistant or refractory severe asthma and/or severe uncontrolled asthma.

5. A method of treating severe uncontrolled asthma in a subject, regardless of eosinophilic phenotype, wherein the subject has had at least two asthma exacerbations during the previous 12 months, the method comprising administering to the subject a compound that binds to or specifically binds to CD131 and neutralises signaling by interleukin (IL) 5 and granulocyte-macrophage colony stimulating factor (GM-CSF) in a subject.

6. The method of any one of claims 1 to 5, wherein the severe asthma is eosinophilic asthma, neutrophilic asthma, paucigranulocytic asthma or mixed granulocytic asthma.

7. The method of any one of claims 1 to 6, wherein the compound that binds to CD131 is administered to the subject as an add-on maintenance therapy, wherein the subject is 12 years and older with severe uncontrolled asthma.

8. The method of any one of claims 1 to 7, wherein the compound that binds to CD131 is administered as a first line maintenance therapy to the subject as an add-on to a standard of care therapy comprising inhaled corticosteroids, long-acting P-agonist and tiotropium, wherein the subject has severe uncontrolled asthma regardless of inflammatory phenotype and biomarker threshold.

9. The method of any one of claims 1 to 8, wherein the subject has, or is suffering from, severe asthma regardless of the eosinophilic phenotype, wherein the severe asthma is uncontrolled despite maximum standard of care therapy, and wherein the subject has had at least 2 asthma exacerbations during the previous 12 months.

10. The method of any one of claims 1 to 9, wherein the subject has, or suffers from, asthma-chronic obstructive pulmonary disease (COPD) overlap (ACO), airway remodelling, fibrosis and/or persistent airflow limitation.

11. The method of any one of claims 1 to 10, wherein administration of the compound reduces, or prevents an increase in, one or more or all of the following:
(xiv) amount of monocytes in the subject's blood;
(xv) amount of neutrophils in the subject's blood;
(xvi) total amount of cells in the bronchoalveolar (BAL) fluid of the subject's lung;
(xvii) macrophage accumulation in the BAL fluid of the subject's lung;
(xviii) neutrophil accumulation in BAL fluid of the subject's lung;
(xix) eosinophil accumulation in the BAL fluid of the subject's lung;
(xx) leukocyte accumulation in the subject's lung;
(xxi) neutrophil accumulation in the subject's lung;
(xxii) eosinophil accumulation in the subject's lung;
(xxiii) interstitial macrophage accumulation in the subject's lung;
(xxiv) alveolar macrophage accumulation in the subject's lung;
(xxv) airway fibrosis of the subject's lung; and/or
(xxvi) inflammation of the subject's lung.

12. The method of any one of claims 1 to 11, wherein administration of the compound that binds to CD131 reduces, or prevents an increase in, one or more or all of the following:
(xii) airway hyper-responsiveness (AHR);
(xiii) baseline airway restriction;
(xiv) collagen fibre deposition in the subject's lung;
(xv) amount of BALF protein in the BAL fluid of the subject's lung;
(xvi) lactate dehydrogenase (LDH) levels in the BAL fluid of the subject's lung;
(xvii) NETosis in the subject's lung;
(xviii) myeloperoxidase activity in the BAL fluid of the subject's lung; and/or
(xix) amount of double stranded DNA (dsDNA) in the BAL fluid of the subject's lung.

13. The method of any one of claims 1 to 12, wherein the compound that binds to CD131 is administered in an amount sufficient to:
(v) reduce asthma exacerbation rates (AER) in the subject by at least 60% (e.g., compared to administration of placebo in the overall population);
(vi) prolong onset to the first exacerbation in the subject;
(vii) improve lung function in the subject as measured by trough forced expiratory volume in one second (FEV₁); and/or
(viii) safely reduce or safely curtail corticosteroid use in the subject.

14. The method of any one of claims 1 to 13, wherein administration of the compound reduces, or prevents an increase in, the level of expression of one or more genes in the subject's lung selected from the group consisting of pro-type I collagen (Col1a1), beta chain cytokine receptor (CSF2RB), Interleukin 13 (IL-13), IL-4, IL-17, IL-12b, interferon gamma (Ifng), C—C Motif Chemokine Ligand 17 (CCL17), Ccl11, matrix metallopeptidase 12 (Mmp12), Mmp13, Thrombospondin 4 (Thbs4), transforming growth factor-beta (Tgfb1), Proteoglycan 2 (Prg2), Cd3d, Cd8a, Cd4, Cd79a, Cd86, chitinase-like 3 (Chil3), arginase 1 (Arg1), eosinophil-associated, ribonuclease A family member 2 (Ear2), histocompatibility 2, class II antigen A, beta 1 (H2-Ab1) and fibromodulin (Fmod).

15. The method of any one of claims 1 to 14, wherein the compound that binds to CD131 and neutralizes signaling by GM-CSF and IL-5 also neutralizes signaling by IL-3.

16. The method of any one of claims 1 to 15, wherein the compound inhibits:
(a) GM-CSF-induced proliferation of TF-1 erythroleukemic cells with an IC₅₀ of at least 460 nM; and/or
(b) IL-5-induced proliferation of TF-1 erythroleukemic cells with an IC₅₀ of at least 1600 nM; and/or
(c) IL-3-induced proliferation of TF-1 erythroleukemic cells with an IC₅₀ of at least 10 nM.

17. The method of any one of claims 1 to 16, wherein the compound is a CD131-binding protein comprising an antigen binding site that:
(a) binds to or specifically binds to an epitope within site 2 of CD131 and neutralizes signaling by interleukin-5 (IL-5) and granulocyte-macrophage colony stimulating factor (GM-CSF);
(b) binds to an epitope formed upon dimerization of two CD131 polypeptides; and
(c) binds to residues within domain 1 of a CD131 polypeptide and residues within domain 4 of another CD131 polypeptide.

18. The method of claim 17, wherein the epitope comprises one or more (or all) of amino acids corresponding to residues 39 and 103 of one CD131 polypeptide and residues 338, 365, 367 and 368 of the other CD131 polypeptide.

19. The method of any one of claims 1 to 18, wherein the compound is a CD131-binding protein that binds to one or more (or all) of the following mutant polypeptide(s):
(i) a mutant polypeptide comprising a sequence set forth in SEQ ID NO: 119;
(ii) a mutant polypeptide comprising a sequence set forth in SEQ ID NO: 123;
(iii) a mutant polypeptide comprising a sequence set forth in SEQ ID NO: 124;
(iv) a mutant polypeptide comprising a sequence set forth in SEQ ID NO: 135;
(v) a mutant polypeptide comprising a sequence set forth in SEQ ID NO: 131;
(vi) a mutant polypeptide comprising a sequence set forth in SEQ ID NO: 136;
(vii) a mutant polypeptide comprising a sequence set forth in SEQ ID NO: 137;
(viii) a mutant polypeptide comprising a sequence set forth in SEQ ID NO: 139;
(ix) a mutant polypeptide comprising a sequence set forth in SEQ ID NO: 145, at a level that is reduced compared to the level of binding of the CD131-binding protein to a polypeptide comprising a sequence set forth in SEQ ID NO: 192.

20. The method of any one of claims 1 to 19, wherein the compound is a CD131-binding protein comprising a Fv.

21. The method of claim 20, wherein the CD131-binding protein comprises:
(i) a single chain Fv fragment (scFv);
(ii) a dimeric scFv (di-scFv);
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fc or a heavy chain constant domain (C_{H}) 2 and/or C_{H}3; or
(x) one of (i) to (viii) linked to albumin or a functional fragment or variants thereof or a protein that binds to albumin; or
(xi) an antibody.

22. The method of any one of claims 1 to 21, wherein the compound is a CD131-binding protein comprising an antigen binding domain of an antibody, wherein the antigen binding domain binds to or specifically binds to CD131, and wherein the CD131-binding protein competitively inhibits binding of antibody 9A2 (comprising a light chain variable region (V_{L}) comprising a sequence set forth in SEQ ID NO: 5 and a heavy chain variable region (V_{H}) comprising a sequence set forth in SEQ ID NO: 20).

23. The method of any one of claims 1 to 22, wherein the compound is a CD131-binding protein comprising an antigen binding domain of an antibody, wherein the antigen binding domain binds to a polypeptide comprising a sequence set forth in SEQ ID NO: 127 with a higher affinity than it does to a polypeptide comprising a sequence set forth in SEQ ID NO: 192.

24. The method of any one of claims 1 to 23, wherein the compound is a CD131-binding protein comprising an antigen binding domain of an antibody, wherein the antigen binding domain comprises a V_{H} and a V_{L}, wherein:
(A) (i) the V_{H} comprises a CDR1 comprising a sequence set forth between amino acids 26-35 of SEQ ID NO: 180, a CDR2 comprising a sequence set forth between amino acids 50-66 of SEQ ID NO: 180 and a CDR3 comprising a sequence set forth between amino acids 99-106 of SEQ ID NO: 180 or the V_{H} comprising a sequence set forth in SEQ ID NO: 180; and
(ii) the V_{L} comprising a CDR1 comprising a sequence set forth between amino acids 24-34 of SEQ ID NO: 177, a CDR2 comprising a sequence set forth between amino acids 44-51 of SEQ ID NO: 177 and a CDR3 comprising a sequence set forth between amino acids 89-97 of SEQ ID NO: 177 or the V_{L} comprising a sequence set forth in SEQ ID NO: 177; or
(B) the V_{H} comprises a sequence set forth in SEQ ID NO: 193 and the V_{L} comprises a sequence set forth in SEQ ID NO: 5.

25. The method of any one of claims 1 to 24, wherein the compound is a CD131-binding protein comprising an antigen binding domain of an antibody, wherein the antigen binding domain comprises:
(i) a V_{H} comprising CDRs 1, 2 and 3 of a sequence set forth in SEQ ID NO: 20 and a V_{L} comprising CDRs 1, 2 and 3 of a sequence set forth in SEQ ID NO: 5;
(ii) a V_{H} comprising a sequence set forth in SEQ ID NO: 20 and a V_{L} comprising a sequence set forth in SEQ ID NO: 5;
(iii) a V_{H} comprising CDRs 1, 2 and 3 of a sequence set forth in SEQ ID NO: 20 and a V_{L} comprising CDRs 1, 2 and 3 of a sequence set forth in SEQ ID NO: 6;
(iv) a V_{H} comprising a sequence set forth in SEQ ID NO: 20 and a V_{L} comprising a sequence set forth in SEQ ID NO: 6;
(v) a V_{H} comprising CDRs 1, 2 and 3 of a sequence set forth in SEQ ID NO: 20 and a V_{L} comprising CDRs 1, 2 and 3 of a sequence set forth in SEQ ID NO: 7;
(vi) a V_{H} comprising a sequence set forth in SEQ ID NO: 20 and a V_{L} comprising a sequence set forth in SEQ ID NO: 7;
(vii) a V_{H} comprising CDRs 1, 2 and 3 of a sequence set forth in SEQ ID NO: 20 and a V_{L} comprising CDRs 1, 2 and 3 of a sequence set forth in SEQ ID NO: 8;
(viii) a V_{H} comprising a sequence set forth in SEQ ID NO: 20 and a V_{L} comprising a sequence set forth in SEQ ID NO: 8;
(ix) a V_{H} comprising CDRs 1, 2 and 3 of a sequence set forth in SEQ ID NO: 20 and a V_{L} comprising CDRs 1, 2 and 3 of a sequence set forth in SEQ ID NO: 9;
(x) a V_{H} comprising a sequence set forth in SEQ ID NO: 20 and a V_{L} comprising a sequence set forth in SEQ ID NO: 9;
(xi) a V_{H} comprising CDRs 1, 2 and 3 of a sequence set forth in SEQ ID NO: 20 and a V_{L} comprising CDRs 1, 2 and 3 of a sequence set forth in SEQ ID NO: 10;
(xii) a V_{H} comprising a sequence set forth in SEQ ID NO: 20 and a V_{L} comprising a sequence set forth in SEQ ID NO: 10;
(xiii) a V_{H} comprising CDRs 1, 2 and 3 of a sequence set forth in SEQ ID NO: 20 and a V_{L} comprising CDRs 1, 2 and 3 of a sequence set forth in SEQ ID NO: 11;
(xiv) a V_{H} comprising a sequence set forth in SEQ ID NO: 20 and a V_{L} comprising a sequence set forth in SEQ ID NO: 11;
(xv) a V_{H} comprising CDRs 1, 2 and 3 of a sequence set forth in SEQ ID NO: 20 and a V_{L} comprising CDRs 1, 2 and 3 of a sequence set forth in SEQ ID NO: 12;
(xvi) a V_{H} comprising a sequence set forth in SEQ ID NO: 20 and a V_{L} comprising a sequence set forth in SEQ ID NO: 12;
(xvii) a V_{H} comprising CDRs 1, 2 and 3 of a sequence set forth in SEQ ID NO: 20 and a V_{L} comprising CDRs 1, 2 and 3 of a sequence set forth in SEQ ID NO: 13;
(xviii) a V_{H} comprising a sequence set forth in SEQ ID NO: 20 and a V_{L} comprising a sequence set forth in SEQ ID NO: 13;
(xix) a V_{H} comprising CDRs 1, 2 and 3 of a sequence set forth in SEQ ID NO: 20 and a V_{L} comprising CDRs 1, 2 and 3 of a sequence set forth in SEQ ID NO: 14;
(xx) a V_{H} comprising a sequence set forth in SEQ ID NO: 20 and a V_{L} comprising a sequence set forth in SEQ ID NO: 14;
(xxi) a V_{H} comprising CDRs 1, 2 and 3 of a sequence set forth in SEQ ID NO: 20 and a V_{L} comprising CDRs 1, 2 and 3 of a sequence set forth in SEQ ID NO: 15;
(xxii) a V_{H} comprising a sequence set forth in SEQ ID NO: 20 and a V_{L} comprising a sequence set forth in SEQ ID NO: 15;
(xxiii) a V_{H} comprising CDRs 1, 2 and 3 of a sequence set forth in SEQ ID NO: 21 and a V_{L} comprising CDRs 1, 2 and 3 of a sequence set forth in SEQ ID NO: 5;
(xxiv) a V_{H} comprising a sequence set forth in SEQ ID NO: 21 and a V_{L} comprising a sequence set forth in SEQ ID NO: 5;
(xxv) a V_{H} comprising CDRs 1, 2 and 3 of a sequence set forth in SEQ ID NO: 22 and a V_{L} comprising CDRs 1, 2 and 3 of a sequence set forth in SEQ ID NO: 5;
(xxvi) a V_{H} comprising a sequence set forth in SEQ ID NO: 22 and a V_{L} comprising a sequence set forth in SEQ ID NO: 5;
(xxvii) a V_{H} comprising CDRs 1, 2 and 3 of a sequence set forth in SEQ ID NO: 23 and a V_{L} comprising CDRs 1, 2 and 3 of a sequence set forth in SEQ ID NO: 5;
(xxviii) a V_{H} comprising a sequence set forth in SEQ ID NO: 23 and a V_{L} comprising a sequence set forth in SEQ ID NO: 5;
(xxix) a V_{H} comprising CDRs 1, 2 and 3 of a sequence set forth in SEQ ID NO: 24 and a V_{L} comprising CDRs 1, 2 and 3 of a sequence set forth in SEQ ID NO: 5;
(xxx) a V_{H} comprising a sequence set forth in SEQ ID NO: 24 and a V_{L} comprising a sequence set forth in SEQ ID NO: 5;
(xxxi) a V_{H} comprising CDRs 1, 2 and 3 of a sequence set forth in SEQ ID NO: 25 and a V_{L} comprising CDRs 1, 2 and 3 of a sequence set forth in SEQ ID NO: 5;
(xxxii) a V_{H} comprising a sequence set forth in SEQ ID NO: 25 and a V_{L} comprising a sequence set forth in SEQ ID NO: 5;
(xxxiii) a V_{H} comprising CDRs 1, 2 and 3 of a sequence set forth in SEQ ID NO: 26 and a V_{L} comprising CDRs 1, 2 and 3 of a sequence set forth in SEQ ID NO: 5;
(xxxiv) a V_{H} comprising a sequence set forth in SEQ ID NO: 26 and a V_{L} comprising a sequence set forth in SEQ ID NO: 5;
(xxxv) a V_{H} comprising CDRs 1, 2 and 3 of a sequence set forth in SEQ ID NO: 27 and a V_{L} comprising CDRs 1, 2 and 3 of a sequence set forth in SEQ ID NO: 5;
(xxxvi) a V_{H} comprising a sequence set forth in SEQ ID NO: 27 and a V_{L} comprising a sequence set forth in SEQ ID NO: 5;
(xxxvii) a V_{H} comprising CDRs 1, 2 and 3 of a sequence set forth in SEQ ID NO: 28 and a V_{L} comprising CDRs 1, 2 and 3 of a sequence set forth in SEQ ID NO: 5;
(xxxviii) a V_{H} comprising a sequence set forth in SEQ ID NO: 28 and a V_{L} comprising a sequence set forth in SEQ ID NO: 5;
(xxxix) a V_{H} comprising CDRs 1, 2 and 3 of a sequence set forth in SEQ ID NO: 29 and a V_{L} comprising CDRs 1, 2 and 3 of a sequence set forth in SEQ ID NO: 5;
(xl) a V_{H} comprising a sequence set forth in SEQ ID NO: 29 and a V_{L} comprising a sequence set forth in SEQ ID NO: 5;
(xli) a V_{H} comprising CDRs 1, 2 and 3 of a sequence set forth in SEQ ID NO: 30 and a V_{L} comprising CDRs 1, 2 and 3 of a sequence set forth in SEQ ID NO: 5;
(xlii) a V_{H} comprising a sequence set forth in SEQ ID NO: 30 and a V_{L} comprising a sequence set forth in SEQ ID NO: 5;
(xliii) a V_{H} comprising CDRs 1, 2 and 3 of a sequence set forth in SEQ ID NO: 31 and a V_{L} comprising CDRs 1, 2 and 3 of a sequence set forth in SEQ ID NO: 5;
(xliv) a V_{H} comprising a sequence set forth in SEQ ID NO: 31 and a V_{L} comprising a sequence set forth in SEQ ID NO: 5;
(xlv) a V_{H} comprising CDRs 1, 2 and 3 of a sequence set forth in SEQ ID NO: 32 and a V_{L} comprising CDRs 1, 2 and 3 of a sequence set forth in SEQ ID NO: 5;
(xlvi) a V_{H} comprising a sequence set forth in SEQ ID NO: 32 and a V_{L} comprising a sequence set forth in SEQ ID NO: 5;
(xlvii) a V_{H} comprising CDRs 1, 2 and 3 of a sequence set forth in SEQ ID NO: 33 and a V_{L} comprising CDRs 1, 2 and 3 of a sequence set forth in SEQ ID NO: 5;
(xlviii) a V_{H} comprising a sequence set forth in SEQ ID NO: 33 and a V_{L} comprising a sequence set forth in SEQ ID NO: 5;
(xlix) a V_{H} comprising CDRs 1, 2 and 3 of a sequence set forth in SEQ ID NO: 34 and a V_{L} comprising CDRs 1, 2 and 3 of a sequence set forth in SEQ ID NO: 5;
(l) a V_{H} comprising a sequence set forth in SEQ ID NO: 34 and a V_{L} comprising a sequence set forth in SEQ ID NO: 5;
(li) a V_{H} comprising CDRs 1, 2 and 3 of a sequence set forth in SEQ ID NO: 35 and a V_{L} comprising CDRs 1, 2 and 3 of a sequence set forth in SEQ ID NO: 5;
(lii) a V_{H} comprising a sequence set forth in SEQ ID NO: 35 and a V_{L} comprising a sequence set forth in SEQ ID NO: 5;
(liii) a V_{H} comprising CDRs 1, 2 and 3 of a sequence set forth in SEQ ID NO: 36 and a V_{L} comprising CDRs 1, 2 and 3 of a sequence set forth in SEQ ID NO: 5;
(liv) a V_{H} comprising a sequence set forth in SEQ ID NO: 36 and a V_{L} comprising a sequence set forth in SEQ ID NO: 5;
(lv) a V_{H} comprising CDRs 1, 2 and 3 of a sequence set forth in SEQ ID NO: 37 and a V_{L} comprising CDRs 1, 2 and 3 of a sequence set forth in SEQ ID NO: 5;
(lvi) a V_{H} comprising a sequence set forth in SEQ ID NO: 37 and a V_{L} comprising a sequence set forth in SEQ ID NO: 5;
(lvii) a V_{H} comprising CDRs 1, 2 and 3 of a sequence set forth in SEQ ID NO: 38 and a V_{L} comprising CDRs 1, 2 and 3 of a sequence set forth in SEQ ID NO: 5;
(lviii) a V_{H} comprising a sequence set forth in SEQ ID NO: 38 and a V_{L} comprising a sequence set forth in SEQ ID NO: 5;
(lix) a V_{H} comprising CDRs 1, 2 and 3 of a sequence set forth in SEQ ID NO: 39 and a V_{L} comprising CDRs 1, 2 and 3 of a sequence set forth in SEQ ID NO: 5;
(lx) a V_{H} comprising a sequence set forth in SEQ ID NO: 39 and a V_{L} comprising a sequence set forth in SEQ ID NO: 5;
(lxi) a V_{H} comprising CDRs 1, 2 and 3 of a sequence set forth in SEQ ID NO: 40 and a V_{L} comprising CDRs 1, 2 and 3 of a sequence set forth in SEQ ID NO: 5;
(lxii) a V_{H} comprising a sequence set forth in SEQ ID NO: 40 and a V_{L} comprising a sequence set forth in SEQ ID NO: 5;
(lxiii) a V_{H} comprising CDRs 1, 2 and 3 of a sequence set forth in SEQ ID NO: 41 and a V_{L} comprising CDRs 1, 2 and 3 of a sequence set forth in SEQ ID NO: 5;
(lxiv) a V_{H} comprising a sequence set forth in SEQ ID NO: 41 and a V_{L} comprising a sequence set forth in SEQ ID NO: 5;
(lxv) a V_{H} comprising CDRs 1, 2 and 3 of a sequence set forth in SEQ ID NO: 42 and a V_{L} comprising CDRs 1, 2 and 3 of a sequence set forth in SEQ ID NO: 5;
(lxvi) a V_{H} comprising a sequence set forth in SEQ ID NO: 42 and a V_{L} comprising a sequence set forth in SEQ ID NO: 5;
(lxvii) a V_{H} comprising CDRs 1, 2 and 3 of a sequence set forth in SEQ ID NO: 43 and a V_{L} comprising CDRs 1, 2 and 3 of a sequence set forth in SEQ ID NO: 5;
(lxviii) a V_{H} comprising a sequence set forth in SEQ ID NO: 43 and a V_{L} comprising a sequence set forth in SEQ ID NO: 5;
(lxix) a V_{H} comprising CDRs 1, 2 and 3 of a sequence set forth in SEQ ID NO: 44 and a V_{L} comprising CDRs 1, 2 and 3 of a sequence set forth in SEQ ID NO: 5;
(lxx) a V_{H} comprising a sequence set forth in SEQ ID NO: 44 and a V_{L} comprising a sequence set forth in SEQ ID NO: 5;
(lxxi) a V_{H} comprising CDRs 1, 2 and 3 of a sequence set forth in SEQ ID NO: 45 and a V_{L} comprising CDRs 1, 2 and 3 of a sequence set forth in SEQ ID NO: 5;
(lxxii) a V_{H} comprising a sequence set forth in SEQ ID NO: 45 and a V_{L} comprising a sequence set forth in SEQ ID NO: 5;
(lxxiii) a V_{H} comprising CDRs 1, 2 and 3 of a sequence set forth in SEQ ID NO: 46 and a V_{L} comprising CDRs 1, 2 and 3 of a sequence set forth in SEQ ID NO: 5;
(lxxiv) a V_{H} comprising a sequence set forth in SEQ ID NO: 46 and a V_{L} comprising a sequence set forth in SEQ ID NO: 5;
(lxxv) a V_{H} comprising CDRs 1, 2 and 3 of a sequence set forth in SEQ ID NO: 47 and a V_{L} comprising CDRs 1, 2 and 3 of a sequence set forth in SEQ ID NO: 5;
(lxxvi) a V_{H} comprising a sequence set forth in SEQ ID NO: 47 and a V_{L} comprising a sequence set forth in SEQ ID NO: 5;
(lxxvii) a V_{H} comprising CDRs 1, 2 and 3 of a sequence set forth in SEQ ID NO: 48 and a V_{L} comprising CDRs 1, 2 and 3 of a sequence set forth in SEQ ID NO: 5;
(lxxviii) a V_{H} comprising a sequence set forth in SEQ ID NO: 48 and a V_{L} comprising a sequence set forth in SEQ ID NO: 5;
(lxxix) a V_{H} comprising CDRs 1, 2 and 3 of a sequence set forth in SEQ ID NO: 49 and a V_{L} comprising CDRs 1, 2 and 3 of a sequence set forth in SEQ ID NO: 5;
(lxxx) a V_{H} comprising a sequence set forth in SEQ ID NO: 49 and a V_{L} comprising a sequence set forth in SEQ ID NO: 5;
(lxxxi) a V_{H} comprising CDRs 1, 2 and 3 of a sequence set forth in SEQ ID NO: 50 and a V_{L} comprising CDRs 1, 2 and 3 of a sequence set forth in SEQ ID NO: 5;
(lxxxii) a V_{H} comprising a sequence set forth in SEQ ID NO: 50 and a V_{L} comprising a sequence set forth in SEQ ID NO: 5;
(lxxxiii) a V_{H} comprising CDRs 1, 2 and 3 of a sequence set forth in SEQ ID NO: 51 and a V_{L} comprising CDRs 1, 2 and 3 of a sequence set forth in SEQ ID NO: 5;
(lxxxiv) a V_{H} comprising a sequence set forth in SEQ ID NO: 51 and a V_{L} comprising a sequence set forth in SEQ ID NO: 5;
(lxxxv) a V_{H} comprising CDRs 1, 2 and 3 of a sequence set forth in SEQ ID NO: 52 and a V_{L} comprising CDRs 1, 2 and 3 of a sequence set forth in SEQ ID NO: 5;
(lxxxvi) a V_{H} comprising a sequence set forth in SEQ ID NO: 52 and a V_{L} comprising a sequence set forth in SEQ ID NO: 5;
(lxxxvii) a V_{H} comprising CDRs 1, 2 and 3 of a sequence set forth in SEQ ID NO: 53 and a V_{L} comprising CDRs 1, 2 and 3 of a sequence set forth in SEQ ID NO: 5;
(lxxxviii) a V_{H} comprising a sequence set forth in SEQ ID NO: 53 and a V_{L} comprising a sequence set forth in SEQ ID NO: 5;
(lxxxix) a V_{H} comprising CDRs 1, 2 and 3 of a sequence set forth in SEQ ID NO: 54 and a V_{L} comprising CDRs 1, 2 and 3 of a sequence set forth in SEQ ID NO: 5;
(xc) a V_{H} comprising a sequence set forth in SEQ ID NO: 54 and a V_{L} comprising a sequence set forth in SEQ ID NO: 5;
(xci) a V_{H} comprising CDRs 1, 2 and 3 of a sequence set forth in SEQ ID NO: 55 and a V_{L} comprising CDRs 1, 2 and 3 of a sequence set forth in SEQ ID NO: 5;
(xcii) a V_{H} comprising a sequence set forth in SEQ ID NO: 55 and a V_{L} comprising a sequence set forth in SEQ ID NO: 5;
(xciii) a V_{H} comprising CDRs 1, 2 and 3 of a sequence set forth in SEQ ID NO: 56 and a V_{L} comprising CDRs 1, 2 and 3 of a sequence set forth in SEQ ID NO: 5;
(xciv) a V_{H} comprising a sequence set forth in SEQ ID NO: 56 and a V_{L} comprising a sequence set forth in SEQ ID NO: 5;
(xcv) a V_{H} comprising CDRs 1, 2 and 3 of a sequence set forth in SEQ ID NO: 57 and a V_{L} comprising CDRs 1, 2 and 3 of a sequence set forth in SEQ ID NO: 5;
(xcvi) a V_{H} comprising a sequence set forth in SEQ ID NO: 57 and a V_{L} comprising a sequence set forth in SEQ ID NO: 5;
(xcvii) a V_{H} comprising CDRs 1, 2 and 3 of a sequence set forth in SEQ ID NO: 58 and a V_{L} comprising CDRs 1, 2 and 3 of a sequence set forth in SEQ ID NO: 5;
(xcviii) a V_{H} comprising a sequence set forth in SEQ ID NO: 58 and a V_{L} comprising a sequence set forth in SEQ ID NO: 5;
(xcix) a V_{H} comprising CDRs 1, 2 and 3 of a sequence set forth in SEQ ID NO: 59 and a V_{L} comprising CDRs 1, 2 and 3 of a sequence set forth in SEQ ID NO: 5;
(c) a V_{H} comprising a sequence set forth in SEQ ID NO: 59 and a V_{L} comprising a sequence set forth in SEQ ID NO: 5;
(ci) a V_{H} comprising CDRs 1, 2 and 3 of a sequence set forth in SEQ ID NO: 60 and a V_{L} comprising CDRs 1, 2 and 3 of a sequence set forth in SEQ ID NO: 5;
(cii) a V_{H} comprising a sequence set forth in SEQ ID NO: 60 and a V_{L} comprising a sequence set forth in SEQ ID NO: 5;
(ciii) a V_{H} comprising CDRs 1, 2 and 3 of a sequence set forth in SEQ ID NO: 61 and a V_{L} comprising CDRs 1, 2 and 3 of a sequence set forth in SEQ ID NO: 5;
(civ) a V_{H} comprising a sequence set forth in SEQ ID NO: 61 and a V_{L} comprising a sequence set forth in SEQ ID NO: 5;
(cv) a V_{H} comprising CDRs 1, 2 and 3 of a sequence set forth in SEQ ID NO: 62 and a V_{L} comprising CDRs 1, 2 and 3 of a sequence set forth in SEQ ID NO: 5;
(cvi) a V_{H} comprising a sequence set forth in SEQ ID NO: 62 and a V_{L} comprising a sequence set forth in SEQ ID NO: 5;
(cvii) a V_{H} comprising CDRs 1, 2 and 3 of a sequence set forth in SEQ ID NO: 63 and a V_{L} comprising CDRs 1, 2 and 3 of a sequence set forth in SEQ ID NO: 5;
(cviii) a V_{H} comprising a sequence set forth in SEQ ID NO: 63 and a V_{L} comprising a sequence set forth in SEQ ID NO: 5;
(cix) a V_{H} comprising CDRs 1, 2 and 3 of a sequence set forth in SEQ ID NO: 64 and a V_{L} comprising CDRs 1, 2 and 3 of a sequence set forth in SEQ ID NO: 5;
(cx) a V_{H} comprising a sequence set forth in SEQ ID NO: 64 and a V_{L} comprising a sequence set forth in SEQ ID NO: 5;
(cxi) a V_{H} comprising CDRs 1, 2 and 3 of a sequence set forth in SEQ ID NO: 65 and a V_{L} comprising CDRs 1, 2 and 3 of a sequence set forth in SEQ ID NO: 5;
(cxii) a V_{H} comprising a sequence set forth in SEQ ID NO: 65 and a V_{L} comprising a sequence set forth in SEQ ID NO: 5;
(cxiii) a V_{H} comprising CDRs 1, 2 and 3 of a sequence set forth in SEQ ID NO: 66 and a V_{L} comprising CDRs 1, 2 and 3 of a sequence set forth in SEQ ID NO: 5;
(cxiv) a V_{H} comprising a sequence set forth in SEQ ID NO: 66 and a V_{L} comprising a sequence set forth in SEQ ID NO: 5;
(cxv) a V_{H} comprising CDRs 1, 2 and 3 of a sequence set forth in SEQ ID NO: 67 and a V_{L} comprising CDRs 1, 2 and 3 of a sequence set forth in SEQ ID NO: 5;
(cxvi) a V_{H} comprising a sequence set forth in SEQ ID NO: 67 and a V_{L} comprising a sequence set forth in SEQ ID NO: 5;
(cxvii) a V_{H} comprising CDRs 1, 2 and 3 of a sequence set forth in SEQ ID NO: 68 and a V_{L} comprising CDRs 1, 2 and 3 of a sequence set forth in SEQ ID NO: 5;
(cxviii) a V_{H} comprising a sequence set forth in SEQ ID NO: 68 and a V_{L} comprising a sequence set forth in SEQ ID NO: 5;
(cxix) a V_{H} comprising CDRs 1, 2 and 3 of a sequence set forth in SEQ ID NO: 69 and a V_{L} comprising CDRs 1, 2 and 3 of a sequence set forth in SEQ ID NO: 5;
(cxx) a V_{H} comprising a sequence set forth in SEQ ID NO: 69 and a V_{L} comprising a sequence set forth in SEQ ID NO: 5;
(cxxi) a V_{H} comprising CDRs 1, 2 and 3 of a sequence set forth in SEQ ID NO: 70 and a V_{L} comprising CDRs 1, 2 and 3 of a sequence set forth in SEQ ID NO: 5;
(cxxii) a V_{H} comprising a sequence set forth in SEQ ID NO: 70 and a V_{L} comprising a sequence set forth in SEQ ID NO: 5;
(cxxiii) a V_{H} comprising CDRs 1, 2 and 3 of a sequence set forth in SEQ ID NO: 71 and a V_{L} comprising CDRs 1, 2 and 3 of a sequence set forth in SEQ ID NO: 5;
(cxxiv) a V_{H} comprising a sequence set forth in SEQ ID NO: 71 and a V_{L} comprising a sequence set forth in SEQ ID NO: 5;
(cxxv) a V_{H} comprising CDRs 1, 2 and 3 of a sequence set forth in SEQ ID NO: 72 and a V_{L} comprising CDRs 1, 2 and 3 of a sequence set forth in SEQ ID NO: 5;
(cxxvi) a V_{H} comprising a sequence set forth in SEQ ID NO: 72 and a V_{L} comprising a sequence set forth in SEQ ID NO: 5;
(cxxvii) a V_{H} comprising CDRs 1, 2 and 3 of a sequence set forth in SEQ ID NO: 73 and a V_{L} comprising CDRs 1, 2 and 3 of a sequence set forth in SEQ ID NO: 5;
(cxxviii) a V_{H} comprising a sequence set forth in SEQ ID NO: 73 and a V_{L} comprising a sequence set forth in SEQ ID NO: 5;
(cxxix) a V_{H} comprising CDRs 1, 2 and 3 of a sequence set forth in SEQ ID NO: 75 and a V_{L} comprising CDRs 1, 2 and 3 of a sequence set forth in SEQ ID NO: 5;
(cxxx) a V_{H} comprising a sequence set forth in SEQ ID NO: 75 and a V_{L} comprising a sequence set forth in SEQ ID NO: 5;
(cxxxi) a V_{H} comprising CDRs 1, 2 and 3 of a sequence set forth in SEQ ID NO: 76 and a V_{L} comprising CDRs 1, 2 and 3 of a sequence set forth in SEQ ID NO: 5;
(cxxxii) a V_{H} comprising a sequence set forth in SEQ ID NO: 76 and a V_{L} comprising a sequence set forth in SEQ ID NO: 5;
(cxxxiii) a V_{H} comprising CDRs 1, 2 and 3 of a sequence set forth in SEQ ID NO: 77 and a V_{L} comprising CDRs 1, 2 and 3 of a sequence set forth in SEQ ID NO: 5;
(cxxxiv) a V_{H} comprising a sequence set forth in SEQ ID NO: 77 and a V_{L} comprising a sequence set forth in SEQ ID NO: 5;
(cxxxv) a V_{H} comprising CDRs 1, 2 and 3 of a sequence set forth in SEQ ID NO: 78 and a V_{L} comprising CDRs 1, 2 and 3 of a sequence set forth in SEQ ID NO: 5;
(cxxxvi) a V_{H} comprising a sequence set forth in SEQ ID NO: 78 and a V_{L} comprising a sequence set forth in SEQ ID NO: 5;
(cxxxvii) a V_{H} comprising CDRs 1, 2 and 3 of a sequence set forth in SEQ ID NO: 79 and a V_{L} comprising CDRs 1, 2 and 3 of a sequence set forth in SEQ ID NO: 5;
(cxxxviii) a V_{H} comprising a sequence set forth in SEQ ID NO: 79 and a V_{L} comprising a sequence set forth in SEQ ID NO: 5;
(cxxxix) a V_{H} comprising CDRs 1, 2 and 3 of a sequence set forth in SEQ ID NO: 80 and a V_{L} comprising CDRs 1, 2 and 3 of a sequence set forth in SEQ ID NO: 5;
(cxl) a V_{H} comprising a sequence set forth in SEQ ID NO: 80 and a V_{L} comprising a sequence set forth in SEQ ID NO: 5;
(cxli) a V_{H} comprising CDRs 1, 2 and 3 of a sequence set forth in SEQ ID NO: 81 and a V_{L} comprising CDRs 1, 2 and 3 of a sequence set forth in SEQ ID NO: 5;
(cxlii) a V_{H} comprising a sequence set forth in SEQ ID NO: 81 and a V_{L} comprising a sequence set forth in SEQ ID NO: 5;
(cxliii) a V_{H} comprising CDRs 1, 2 and 3 of a sequence set forth in SEQ ID NO: 82 and a V_{L} comprising CDRs 1, 2 and 3 of a sequence set forth in SEQ ID NO: 5;
(cxliv) a V_{H} comprising a sequence set forth in SEQ ID NO: 82 and a V_{L} comprising a sequence set forth in SEQ ID NO: 5;
(cxlv) a V_{H} comprising CDRs 1, 2 and 3 of a sequence set forth in SEQ ID NO: 83 and a V_{L} comprising CDRs 1, 2 and 3 of a sequence set forth in SEQ ID NO: 5;
(cxlvi) a V_{H} comprising a sequence set forth in SEQ ID NO: 83 and a V_{L} comprising a sequence set forth in SEQ ID NO: 5;
(cxlvii) a V_{H} comprising CDRs 1, 2 and 3 of a sequence set forth in SEQ ID NO: 84 and a V_{L} comprising CDRs 1, 2 and 3 of a sequence set forth in SEQ ID NO: 5;
(cxlviii) a V_{H} comprising a sequence set forth in SEQ ID NO: 84 and a V_{L} comprising a sequence set forth in SEQ ID NO: 5;
(cxlix) a V_{H} comprising CDRs 1, 2 and 3 of a sequence set forth in SEQ ID NO: 85 and a V_{L} comprising CDRs 1, 2 and 3 of a sequence set forth in SEQ ID NO: 5;
(cl) a V_{H} comprising a sequence set forth in SEQ ID NO: 85 and a V_{L} comprising a sequence set forth in SEQ ID NO: 5;
(cli) a V_{H} comprising CDRs 1, 2 and 3 of a sequence set forth in SEQ ID NO: 86 and a V_{L} comprising CDRs 1, 2 and 3 of a sequence set forth in SEQ ID NO: 5;
(clii) a V_{H} comprising a sequence set forth in SEQ ID NO: 86 and a V_{L} comprising a sequence set forth in SEQ ID NO: 5;
(cliii) a V_{H} comprising CDRs 1, 2 and 3 of a sequence set forth in SEQ ID NO: 87 and a V_{L} comprising CDRs 1, 2 and 3 of a sequence set forth in SEQ ID NO: 5;
(cliv) a V_{H} comprising a sequence set forth in SEQ ID NO: 87 and a V_{L} comprising a sequence set forth in SEQ ID NO: 5;
(clv) a V_{H} comprising CDRs 1, 2 and 3 of a sequence set forth in SEQ ID NO: 88 and a V_{L} comprising CDRs 1, 2 and 3 of a sequence set forth in SEQ ID NO: 5;
(clvi) a V_{H} comprising a sequence set forth in SEQ ID NO: 88 and a V_{L} comprising a sequence set forth in SEQ ID NO: 5;
(clvii) a V_{H} comprising CDRs 1, 2 and 3 of a sequence set forth in SEQ ID NO: 89 and a V_{L} comprising CDRs 1, 2 and 3 of a sequence set forth in SEQ ID NO: 5;
(clviii) a V_{H} comprising a sequence set forth in SEQ ID NO: 89 and a V_{L} comprising a sequence set forth in SEQ ID NO: 5;
(clix) a V_{H} comprising CDRs 1, 2 and 3 of a sequence set forth in SEQ ID NO: 90 and a V_{L} comprising CDRs 1, 2 and 3 of a sequence set forth in SEQ ID NO: 5;
(clx) a V_{H} comprising a sequence set forth in SEQ ID NO: 90 and a V_{L} comprising a sequence set forth in SEQ ID NO: 5;
(clxi) a V_{H} comprising CDRs 1, 2 and 3 of a sequence set forth in SEQ ID NO: 91 and a V_{L} comprising CDRs 1, 2 and 3 of a sequence set forth in SEQ ID NO: 5;
(clxii) a V_{H} comprising a sequence set forth in SEQ ID NO: 91 and a V_{L} comprising a sequence set forth in SEQ ID NO: 5;
(clxiii) a V_{H} comprising CDRs 1, 2 and 3 of a sequence set forth in SEQ ID NO: 92 and a V_{L} comprising CDRs 1, 2 and 3 of a sequence set forth in SEQ ID NO: 5;
(clxiv) a V_{H} comprising a sequence set forth in SEQ ID NO: 92 and a V_{L} comprising a sequence set forth in SEQ ID NO: 5;
(clxv) a V_{H} comprising CDRs 1, 2 and 3 of a sequence set forth in SEQ ID NO: 93 and a V_{L} comprising CDRs 1, 2 and 3 of a sequence set forth in SEQ ID NO: 5;
(clxvi) a V_{H} comprising a sequence set forth in SEQ ID NO: 93 and a V_{L} comprising a sequence set forth in SEQ ID NO: 5;
(clxvii) a V_{H} comprising CDRs 1, 2 and 3 of a sequence set forth in SEQ ID NO: 94 and a V_{L} comprising CDRs 1, 2 and 3 of a sequence set forth in SEQ ID NO: 5;
(clxviii) a V_{H} comprising a sequence set forth in SEQ ID NO: 94 and a V_{L} comprising a sequence set forth in SEQ ID NO: 5;
(clxix) a V_{H} comprising CDRs 1, 2 and 3 of a sequence set forth in SEQ ID NO: 95 and a V_{L} comprising CDRs 1, 2 and 3 of a sequence set forth in SEQ ID NO: 5;
(clxx) a V_{H} comprising a sequence set forth in SEQ ID NO: 95 and a V_{L} comprising a sequence set forth in SEQ ID NO: 5;
(clxxi) a V_{H} comprising CDRs 1, 2 and 3 of a sequence set forth in SEQ ID NO: 96 and a V_{L} comprising CDRs 1, 2 and 3 of a sequence set forth in SEQ ID NO: 5;
(clxxii) a V_{H} comprising a sequence set forth in SEQ ID NO: 96 and a V_{L} comprising a sequence set forth in SEQ ID NO: 5;
(clxxiii) a V_{H} comprising CDRs 1, 2 and 3 of a sequence set forth in SEQ ID NO: 97 and a V_{L} comprising CDRs 1, 2 and 3 of a sequence set forth in SEQ ID NO: 5;
(clxxiv) a V_{H} comprising a sequence set forth in SEQ ID NO: 97 and a V_{L} comprising a sequence set forth in SEQ ID NO: 5;
(clxxv) a V_{H} comprising CDRs 1, 2 and 3 of a sequence set forth in SEQ ID NO: 98 and a V_{L} comprising CDRs 1, 2 and 3 of a sequence set forth in SEQ ID NO: 5;
(clxxvi) a V_{H} comprising a sequence set forth in SEQ ID NO: 98 and a V_{L} comprising a sequence set forth in SEQ ID NO: 5;
(clxxvii) a V_{H} comprising CDRs 1, 2 and 3 of a sequence set forth in SEQ ID NO: 99 and a V_{L} comprising CDRs 1, 2 and 3 of a sequence set forth in SEQ ID NO: 5;
(clxxviii) a V_{H} comprising a sequence set forth in SEQ ID NO: 99 and a V_{L} comprising a sequence set forth in SEQ ID NO: 5;
(clxxix) a V_{H} comprising CDRs 1, 2 and 3 of a sequence set forth in SEQ ID NO: 100 and a V_{L} comprising CDRs 1, 2 and 3 of a sequence set forth in SEQ ID NO: 5;
(clxxx) a V_{H} comprising a sequence set forth in SEQ ID NO: 100 and a V_{L} comprising a sequence set forth in SEQ ID NO: 5;
(clxxxi) a V_{H} comprising CDRs 1, 2 and 3 of a sequence set forth in SEQ ID NO: 101 and a V_{L} comprising CDRs 1, 2 and 3 of a sequence set forth in SEQ ID NO: 5;
(clxxxii) a V_{H} comprising a sequence set forth in SEQ ID NO: 101 and a V_{L} comprising a sequence set forth in SEQ ID NO: 5;
(clxxxiii) a V_{H} comprising CDRs 1, 2 and 3 of a sequence set forth in SEQ ID NO: 102 and a V_{L} comprising CDRs 1, 2 and 3 of a sequence set forth in SEQ ID NO: 5;
(clxxxiv) a V_{H} comprising a sequence set forth in SEQ ID NO: 102 and a V_{L} comprising a sequence set forth in SEQ ID NO: 5;
(clxxxv) a V_{H} comprising CDRs 1, 2 and 3 of a sequence set forth in SEQ ID NO: 103 and a V_{L} comprising CDRs 1, 2 and 3 of a sequence set forth in SEQ ID NO: 5;
(clxxxvi) a V_{H} comprising a sequence set forth in SEQ ID NO: 103 and a V_{L} comprising a sequence set forth in SEQ ID NO: 5;
(clxxxvii) a V_{H} comprising CDRs 1, 2 and 3 of a sequence set forth in SEQ ID NO: 104 and a V_{L} comprising CDRs 1, 2 and 3 of a sequence set forth in SEQ ID NO: 5;
(clxxxviii) a V_{H} comprising a sequence set forth in SEQ ID NO: 104 and a V_{L} comprising a sequence set forth in SEQ ID NO: 5;
(clxxxix) a V_{H} comprising CDRs 1, 2 and 3 of a sequence set forth in SEQ ID NO: 105 and a V_{L} comprising CDRs 1, 2 and 3 of a sequence set forth in SEQ ID NO: 5;
(cxc) a V_{H} comprising a sequence set forth in SEQ ID NO: 105 and a V_{L} comprising a sequence set forth in SEQ ID NO: 5;
(cxci) a V_{H} comprising CDRs 1, 2 and 3 of a sequence set forth in SEQ ID NO: 106 and a V_{L} comprising CDRs 1, 2 and 3 of a sequence set forth in SEQ ID NO: 5;
(cxcii) a V_{H} comprising a sequence set forth in SEQ ID NO: 106 and a V_{L} comprising a sequence set forth in SEQ ID NO: 5;
(cxciii) a V_{H} comprising CDRs 1, 2 and 3 of a sequence set forth in SEQ ID NO: 107 and a V_{L} comprising CDRs 1, 2 and 3 of a sequence set forth in SEQ ID NO: 5;
(cxciv) a V_{H} comprising a sequence set forth in SEQ ID NO: 107 and a V_{L} comprising a sequence set forth in SEQ ID NO: 5;
(cxcv) a V_{H} comprising CDRs 1, 2 and 3 of a sequence set forth in SEQ ID NO: 108 and a V_{L} comprising CDRs 1, 2 and 3 of a sequence set forth in SEQ ID NO: 5;
(cxcvi) a V_{H} comprising a sequence set forth in SEQ ID NO: 108 and a V_{L} comprising a sequence set forth in SEQ ID NO: 5;
(cxcvii) a V_{H} comprising CDRs 1, 2 and 3 of a sequence set forth in SEQ ID NO: 109 and a V_{L} comprising CDRs 1, 2 and 3 of a sequence set forth in SEQ ID NO: 5;
(cxcviii) a V_{H} comprising a sequence set forth in SEQ ID NO: 109 and a V_{L} comprising a sequence set forth in SEQ ID NO: 5;
(cxcix) a V_{H} comprising CDRs 1, 2 and 3 of a sequence set forth in SEQ ID NO: 110 and a V_{L} comprising CDRs 1, 2 and 3 of a sequence set forth in SEQ ID NO: 5;
(cc) a V_{H} comprising a sequence set forth in SEQ ID NO: 110 and a V_{L} comprising a sequence set forth in SEQ ID NO: 5;
(cci) a V_{H} comprising CDRs 1, 2 and 3 of a sequence set forth in SEQ ID NO: 111 and a V_{L} comprising CDRs 1, 2 and 3 of a sequence set forth in SEQ ID NO: 5;
(ccii) a V_{H} comprising a sequence set forth in SEQ ID NO: 111 and a V_{L} comprising a sequence set forth in SEQ ID NO: 5;
(cciii) a V_{H} comprising CDRs 1, 2 and 3 of a sequence set forth in SEQ ID NO: 112 and a V_{L} comprising CDRs 1, 2 and 3 of a sequence set forth in SEQ ID NO: 5;
(cciv) a V_{H} comprising a sequence set forth in SEQ ID NO: 112 and a V_{L} comprising a sequence set forth in SEQ ID NO: 5;
(ccv) a V_{H} comprising CDRs 1, 2 and 3 of a sequence set forth in SEQ ID NO: 113 and a V_{L} comprising CDRs 1, 2 and 3 of a sequence set forth in SEQ ID NO: 5;
(ccvi) a V_{H} comprising a sequence set forth in SEQ ID NO: 113 and a V_{L} comprising a sequence set forth in SEQ ID NO: 5;
(ccvii) a V_{H} comprising CDRs 1, 2 and 3 of a sequence set forth in SEQ ID NO: 114 and a V_{L} comprising CDRs 1, 2 and 3 of a sequence set forth in SEQ ID NO: 5;
(ccviii) a V_{H} comprising a sequence set forth in SEQ ID NO: 114 and a V_{L} comprising a sequence set forth in SEQ ID NO: 5;
(ccix) a V_{H} comprising CDRs 1, 2 and 3 of a sequence set forth in SEQ ID NO: 115 and a V_{L} comprising CDRs 1, 2 and 3 of a sequence set forth in SEQ ID NO: 5;
(ccx) a V_{H} comprising a sequence set forth in SEQ ID NO: 115 and a V_{L} comprising a sequence set forth in SEQ ID NO: 5;
(ccxi) a V_{H} comprising CDRs 1, 2 and 3 of a sequence set forth in SEQ ID NO: 116 and a V_{L} comprising CDRs 1, 2 and 3 of a sequence set forth in SEQ ID NO: 5; or
(ccxii) a V_{H} comprising a sequence set forth in SEQ ID NO: 116 and a V_{L} comprising a sequence set forth in SEQ ID NO: 5.

26. The method of any one of claims 1 to 25, wherein the compound is an antibody comprising:
(i) a V_{H} comprising CDRs 1, 2 and 3 of a sequence set forth in SEQ ID NO: 64 and a V_{L} comprising CDRs 1, 2 and 3 of a sequence set forth in SEQ ID NO: 5; or
(ii) a V_{H} comprising a sequence set forth in SEQ ID NO: 64 and a V_{L} comprising a sequence set forth in SEQ ID NO: 5.

27. The method of any one of claims 1 to 26, wherein the method further comprises determining a peripheral blood eosinophil and/or neutrophil count and/or a peripheral-blood differential eosinophil and/or neutrophil count in the subject and/or proportion of eosinophils and/or neutrophils in the subject's sputum.

28. The method of claim 27, wherein the proportion of eosinophils in the subject's sputum is at least 3% and/or the proportion of neutrophils in the subject's sputum is at least 61%.

29. The method of any one of claims 1 to 28, wherein the subject is 12 years of age or older.
